(19) United States
(12) Patent Application Publication
(10) Pub. No.: US 2022/0241314 A1
(43) Pub. Date: Aug. 4, 2022

(54) COMPOSITIONS AND METHODS FOR TREATMENT OF HEPATITIS B VIRUS INFECTION

(71) Applicant: University of Washington, Seattle, WA (US)

(72) Inventors: Michael J. Gale, JR., Seattle, WA (US); Sooyoung Lee, Seattle, WA (US)

(73) Assignee: University of Washington, Seattle, WA (US)

(21) Appl. No.: 17/710,783

(22) Filed: Mar. 31, 2022

Related U.S. Application Data
(63) Continuation of application No. PCT/US2020/053606, filed on Sep. 30, 2020.
(60) Provisional application No. 62/909,321, filed on Oct. 2, 2019.

(51) Int. Cl. A61K 31/7088; A61K 31/428; A61K 31/506; A61K 31/675; A61K 31/522; A61K 31/7072; A61P 31/20

(52) U.S. Cl. CPC A61K 31/7088; A61K 31/428; A61K 31/506; A61P 31/20; A61K 31/522; A61K 31/7072; A61K 31/675

(57) ABSTRACT

The disclosure provides compositions and methods for suppressing Hepatitis B virus (HBV) in an infected cell. Exemplary methods comprise contacting the infected cell with one or more agents that induce interferon regulatory factor 3 (IRF3) activation in the infected cell. In some embodiments, the one or more agents comprises pathogen-associated molecular pattern (PAMP)-containing nucleic acid molecule, a small molecule agent (e.g., a benzothiazol-derivative molecule), or a combination thereof. In some embodiments, the method further comprises contacting the infected cell with a NRTI. The method can be an in vivo method of treating a subject with HBV infection, comprising administering therapeutically relevant amounts of one or more agents formulated in one or more therapeutically effect compositions. Exemplary compositions are formulated to treat a hepatitis B virus (HBV) infection in a subject, comprising: a RIG-I agonist, a vehicle for intracellular delivery, and a pharmaceutically acceptable carrier.

### CROSS-REFERENCE TO RELATED APPLICATION

This application claims the benefit of U.S. Provisional Application No. 62/909,321, filed Oct. 2, 2019, the entire contents of which are incorporated herein by reference.

### STATEMENT OF GOVERNMENT LICENSE RIGHTS

This invention was made with Government support under Grant Nos. R01 AI118916 and R01 AI127463, awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 72750_Sequence_Listing_final_2020-09-28,txt. The text file is 31 KB; was created on Sep. 28, 2020; and is being submitted via EFS-Web with the filing of the specification.

### BACKGROUND

Hepatitis B virus (HBV) is a global public health problem with more than 250 million people chronically infected worldwide. Chronic HBV infection is a leading cause of liver disease including liver cirrhosis, hepatocellular carcinoma (HCC), and liver failure such that HBV infection causes over 700,000 deaths annually [WHO, 2017].

HBV is a small, hepatotropic DNA virus that replicates in part through reverse transcription. HBV specifically enters hepatocytes through the sodium-taurocholate cotransporting polypeptide (NTCP) receptor to replicate and produce virion. After entry into the hepatocyte cytosol, the viral nucleocapsid is translocated to the nucleus for disassembly and release of the viral relaxed circular (RC) DNA. In the nucleus, the RC DNA is converted to covalently closed circular DNA (cccDNA) that is a long-lived viral mini-chromosome serving as the main template for the synthesis of all HBV RNA transcripts including pregenomic (pg) RNA, pre-S, S, and X viral RNAs. After synthesis the mature nucleocapsid-containing RC DNA acquires an envelope via budding at the endoplasmic reticulum (ER) and then produces progeny virion. A portion of the pool of mature HBV nucleocapsids is used to facilitate further cccDNA synthesis in a process called the intracellular amplification pathway. This process facilitates the pool of cccDNA to be maintained as a steady-state population of 3-50 molecules per cell marking chronic infection. Removal of cccDNA from the liver either through its eradication from infected cells or depletion of infected cells is considered to be essential for HBV cure.

Current treatments for chronic HBV rely on two classes of therapy, including (i) nucleot(s)ide analogs (NAs), which inhibit viral reverse transcriptase and DNA polymerase function, and (ii) pegylated interferon alpha (peg-IFNα) therapy that induces innate immune defenses for suppression of HBV antigen production. Although these therapies can suppress active viral replication, reduce cccDNA levels, and can slow disease progression, they do not eliminate the nuclear pool of cccDNA, and are associated with significant side-effects in treated patients. The persistence of cccDNA is established to be 6-22 weeks in vivo in which lifelong treatment with antiviral therapy is required for a majority of patients to continuously suppress viral replication. Problematically, IFN-based therapy for chronic HBV is poorly tolerated and only a low frequency of treated patients show complete loss of HBsAg that defines clinical HBV cure.

Accordingly, despite the development of suppressive HPV therapies, a need remains for effective therapeutics and treatment strategies that can specifically eradicate cccDNA, thereby leading to a more complete and functional cure and avoiding detrimental effects of extended suppressive therapies. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method for suppressing hepatitis B virus (HBV) covalently-closed-circular DNA (cccDNA) levels in an infected cell. The method comprises contacting the infected cell with an agent that induces interferon regulatory factor 3 (IRF3) activation in the infected cell. “Suppressing cccDNA”can comprise inhibiting cccDNA formation in the infected cell or reducing the stability of existing cccDNA in the infected cell.

In some embodiments, the agent induces IRF3 activation by inducing a retinoic acid-inducible gene I (RIG-I)-like receptor (RLR) signaling pathway. In some embodiments, the agent is or comprises a nucleic acid molecule comprising a pathogen-associated molecular pattern (PAMP), wherein the PAMP comprises: a 5′ arm region comprising a terminal triphosphate; a poly uracil core comprising at least 8 contiguous uracil residues; and a 3′ arm region comprising at least 8 nucleic acid residues, wherein the 5″ most nucleic acid residue of the 3′ arm region is not a uracil, and wherein the 3′ arm region is at least 30% uracil residues. In some embodiments, the agent is a small molecule agent. In some embodiments, the small molecule agent is or comprises a benzothiazol-derivative molecule, such as a small molecule agent comprising the chemical formula N-(6-benzamido-1,3-benzothiazol-2-yl)naphthalene-2-carboxamide. In some embodiments, the method comprises contacting the cell with a combination of a nucleic acid molecule comprising a pathogen-associated molecular pattern (PAMP) and a small molecule agent (e.g., a benzothiazol-derivative molecule, e.g., N-(6-benzamido-1,3-benzothiazol-2-yl)naphthalene-2-carboxamide).

In some embodiments, the method further comprises contacting the cell with a nucleoside reverse transcriptase inhibitor (NRTI). In some embodiments, the NRTI is selected from Lamivudine, Adefovir dipivoxil, Entecavir, Telbivudine, Tenofovir, Tenofovir alafenamide (TAF). Clevudine, Besivo, Zadaxin, Remdesivir, and the like.

In some embodiments, the cell is a hepatocyte.

In another aspect, the disclosure provides a method of treating or preventing a hepatitis B Virus (HBV) infection in a subject in need thereof. The method comprises administering to the subject a therapeutically effective amount of composition that induces interferon regulatory factor 3 (IRF3) activation in infected cells of the subject.

In some embodiments, the composition comprises a nucleic acid molecule comprising a pathogen-associated molecular pattern (PAMP), wherein the PAMP comprises: a 5′ arm region comprising a terminal triphosphate; a poly uracil core comprising at least 8 contiguous uracil residues; and a 3′ arm region comprising at least 8 nucleic acid residues, wherein the 5′ most nucleic acid residue of the 3′ arm region is not a uracil and wherein the 3′ arm region is at least 30% uracil residues. In some embodiments, the agent is a small molecule agent that induces RIG-I signaling. In some embodiments, the small molecule agent is or comprises a benzothiazol-derivative molecule, such as a small molecule agent comprises the chemical formula N-(6-benzamido-1,3-benzothiazol-2-yl)naphthalene-2-carboxamide. In some embodiments, the method comprises administering to the subject therapeutically effective amounts of the nucleic acid molecule comprising a pathogen-associated molecular pattern (PAMP) and the small molecule agent in combination or coordination.

In some embodiments, the method further comprises administering the subject a nucleoside reverse transcriptase inhibitor (NRTI). In some embodiments, the NRTI is selected from Lamivudine, Adefovir dipivoxil, Entecavir, Telbivudine, Tenofovir, Tenofovir alafenamide (TAF), Clevudine, Besivo, Zadaxin, Remdesivir, and the like. The NRTI can be administered in combination or coordination with the one or more agents that induce(s) interferon regulatory factor 3 (IRF3) activation in infected cells of the subject.

In another aspect, the disclosure provides a composition for treating a hepatitis B virus (HBV) infection in a subject comprising: a RIG-I agonist, a vehicle for intracellular delivery, and a pharmaceutically acceptable carrier.

In some embodiments, the RIG-I agonist is or comprises a nucleic acid molecule comprising a pathogen-associated molecular pattern (PAMP), wherein the PAMP comprises: a 5′ arm region comprising a terminal triphosphate; a poly uracil core comprising at least 8 contiguous uracil residues; and a 3′ arm region comprising at least 8 nucleic acid residues, wherein the 5′ most nucleic acid residue of the 3′ arm region is not a uracil and wherein the 3′ arm region is at least 30% uracil residues. In some embodiments, the RIG-I agonist is or comprises is a small molecule agent. In some embodiments, the small molecule agent is or comprises a benzothiazol-derivative molecule, such as a small molecule agent comprising the chemical formula N-(6 -benzamido-1,3-benzothiazol-2-yl)naphthalene-2-carboxamide. In some embodiments, the composition comprises a combination of the nucleic acid molecule comprising a pathogen-associated molecular pattern (PAMP) and the small molecule agent (e.g., benzothiazol-derivative molecule, e.g., comprising the chemical formula N-(6-benzamido -1,3-benzothiazol-2-yl)naphthalene-2-carboxamide). In some embodiments, the composition further comprises a nucleoside reverse transcriptase inhibitor (NRTI). In some embodiments, the NRTI is selected from Lamivudine, Adefovir dipivoxil, Entecavir, Telbivudine, Tenofovir, Tenofovir alafenamide (TAF), Clevudine, Besivo, Zadaxin, Remdesivir, and the like. In some embodiments, the vehicle is a liposome, nanocapsule, nanoparticle, exosome, microparticle, microsphere, lipid particle, vesicle, and the like, configured for the introduction of the RIG-I agonist into target host cells infected with HBV.

In another aspect, the disclosure provides a method of treating a subject with a hepatitis B virus (HBV) infection, comprising administering to the subject a therapeutically effective amount of the compositions disclosed herein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1D illustrate that the F7 small molecule and poly-U/UC PAMP differentially induce innate immune genes. (1A) Structure of F7 and sequence a representative poly-U/UC PAMP-RNA (SEQ ID NO:1). (1B) Immunofluorescence analysis of IRF3 translocation, HepG2-hNTCP cells were cultured in medium containing 2.5% DMSO, infected with Sendai virus (SerV; 10 HAU/ml), or treated with F7 (10 μM), X-RNA (200 ng/ml in liposome), or poly-U/UC PAMP (200 ng/ml in liposome) for 24 hours. Cells were fixed with 3% paraformaldehyde, stained by using mouse anti-hNTCP (green), rabbit anti-IR F3 (red) antibodies and counterstained with DAPI (blue). Scale bars represent 20 μm. (1C) F7 and poly-U/UC induce IRF3 activation and innate immune gene expression. Sendai virus, F7, X-RNA and poly-U/UC PAMP were administered to HepG2-hNTCP and dHepaRG, as indicated for 24 and 48 hours. The cell lysates were resolved by SDS-PAGE and then subjected to immunoblotting. The levels of protein expression by the p-IRF3 (S386 phosphorylation active form of IRF3), IRF3 (total IRF3), and IFIT1 relative to the expression level of tubulin were determined using respective antibodies, (1D) Gene expression analysis. HepG2-hNTCP cells were administered with SenV, F7, X-RNA and poly-U/UC-RNA as described above for 24 and 72 hours, then total cellular RNA was purified from harvested cells. The levels of expression of innate immune genes IFIT1, CXCL10, IFITM1, RSAD2, RIG-I, MDA5, SAMHD1, APOBEC3A, APOBEC3G, IFN-α, IFN-β, and IFN-λ3 were measured by qRT-PCR and normalized to the level of GAPDH expression. Each are shown as the mean -fold induction over that achieved with 2.5% DMSO treatment from three independent experiments.

FIGS. 2A-2E illustrate the therapeutic suppression of cccDNA formation. (2A) Scheme of infection and treatment (upper) and Southern blot analysis (lower) to measure protein-free DNA including cccDNA. The scheme illustrates the schedule with HBV inoculum (1000 Geq/cell) and administration of Cyclosporin A (CsA), F7, X-RNA and poly-U/UC PAMP. 2.5% DMSO was added to the medium at 1 day post infection (Dpi). cccDNA and PF RC DNA were harvested from HepG2-hNTCP cells using Hirt extraction method and analyzed by Southern blot analysis using an HBV-specific DNA probe. Viral protein-free DNAs (protein-free Relaxed Circular DNA [PF-RC DNA] and cccDNA) are noted. (2B and 2C) HepG2-hNTCP 2(B) or differentiated HepaRG cells (2C) were infected with HBV at moi of 1000 Geq/cell, and administered CsA (10 μM) or F7, in the indicated concentrations. For upper panels of (2B) and (2C), DNAs were isolated after Hirt extraction at 3 dpi and subjected to Southern blot analysis using as HBV-DNA probe. For lower panels of (2B) and (2C), the inhibitor effects of F7 on cccDNA formation were measured in HepG2-hNTCP and dHepaRG cells, respectively, using RT-qPCR analysis. (2D and 2E) HepG2-hNTCP (2D) or dHepaRG cells (2E) were infected with HBV at an moi of 1000 Geq/cell, and administered CsA (10 μM), X-RNA (100 ng/ml), or poly-U/UC PAMP in the indicated concentrations. For upper panels of (2D) and (2E), DNAs were analyzed using Southern blot analysis. For lower panels of (2D) and (2E), DNAs were analyzed by RT-qPCR. IC₉₀, and IC₅₀ values were calculated based on the decline of the cccDNA relative to DMSO treated controls. IC₉₀, and IC₅₀ values are the average of three experiments±one standard deviation. Cytotoxicity was determined by measuring cellular ATP content as a measure of cell viability using the CellTiter-Glo™ reagent CC₅₀ values shown are the average of three experiments±one standard deviation. Positions of mass markers are indicated on each Southern blot.

FIGS. 3A-3F illustrate that F7 and poly-U/UC PAMP suppress de novo HBV cccDNA synthesis. (3A) Upper: Infection and poly-U/UC PAMP treatment schedule. Cells were treated with 100 ng/ml of poly-U/UC PAMP either for 24 hours before (Pre), for 48 hours after HBV infection (Post), or for 72 hours (pre/post). At 3 dpi cells were harvested and Hirt extracts were prepared (3B) HepG2-hNTCP cells and (3C) dHepaRG cells were analyzed by Southern blot (upper) and RT-qPCR (lower). For RT-qPCR. anal sis, values from DMSO treated control were set to 100% respectively, and data are shown as mean±standard deviations (SD) percentage of cccDNA in control samples. ***P<0.005, and ns=non-significant. (3D) HBV infection and F7 treatment schedule. Cells were treated with F7 (10 μM) either for 24 hours before (Pre), 24 hours during the time of infection (Co), d8 hours after infection (Post), or 96 hours pre/co/post). At 3 Dpi, the cells were harvested and. Hirt extracts prepared HepG2-hNTCP (3E) and dHepaRG cells (3F). Upper panels show Southern blot analysis. Lower panels show or RT-qPCR analysis and percent of cccDNA remaining in treated cells compared to control DMS)-treated cells. Values from DMSO-treated control cultures were set to 100%. Data was presented as mean±standard deviations (SD), *P<0.01, **P<0.005, ***P<0.001, ****P<0.0001 and ns=non-significant. CsA served as a treatment control. For Southern blots the position of mass markers are indicated.

FIG. 4A-4D illustrate subcellular compartment analysis of antiviral activity. (4A) and (4C) Scheme of HBV infection with F7 treatment (4A) or poly-U/UC PAMP treatment (4C). HepG2-hNTCP cultures were inoculated with HBV at moi 1000 Geq/cell for 24 hr. On treatment day 0 the cultures received F7 (10 μM) or poly-U/UC PAMP (100 ng/ml). Cells were harvested for production of Hirt extracts at each time point shown through three days. Parallel cultures were harvested for Western blot analysis to monitor Lamin B1 and Calnexin as markers of whole cell lysate and cytosol respectively. (4B) and (4D) DNA was analyzed by Southern blot using HBV-specific DNA probe. Viral protein-free DNAs (protein-free Relaxed Circular DNA [PF-RC DNA]and cccDNA) are indicated. The positions of mass markers are indicated. Lower panels show Western blot of Lamin B1 and Calnexin abundance.

FIG. 5A-5F illustrate that F7 and poly-U/UC PAMP treatment directs HBV cccDNA decay. (5A) HBV infection and treatment schedule. On day 0 HepG2-hNTCP cells were infected with HBV at moi of 1000 Geq/cell, incubated for 24 hr, and media was replaced. On day three the cells were harvested or treated with DMSO, ETV (500 nM), F7 (10 μM), poly-U/UC (100 ng/ml), or with ETC combination with F7 or poly-U/UC, PAMP. Cells were harvested at the indicated points over a 20-day time course. (5B), (5C) DNA was isolated by Hirt extract and subjected to Southern blot analysis using an HBV-specific DNA probe. Percentage values below each lane indicate the relative amount of cccDNA compared to day three control prior to each treatment. The positions of mass markers are indicated. (5D) cccDNA levels were measured by RT-qPCR. Relative cccDNA values to mitochondrial DNA (MT-CO3) are shown. Statistical significance was determined using Student's t test. Data are presented as mean standard deviation (SD), *P<0.01, **P<0.005, ***P<0.001 and ns=non-significant (E) Half-life of cccDNA from RT-qPCR analyses was estimated from the simultaneous fitting of three replicates under each treatment strategy. C(t) is the % values of cccDNA at time ‘t’ post-treatment, C(0) is the % values of cccDNA at the start of the treatment. (5F) HBsAg across the time course for each treatment series. Statistical significance as determined using Student's t test. Data are presented as mean standard deviations (SD), **P<0.005, and ns=non-significant.

FIGS. 6A-6D illustrate that F7 and poly-U/UC PAMP specifically signal IRF3 activation through RIG-I to suppress HBV cccDNA. (6A) F7 was administered for 24 hours to HepG2-hNTCP-NT (expressing non-targeting guide RNA), RKO (RIG-I knockout expressing RIG-I-targeting guide RNA), and MKO (MDA5-knock out expressing MDA5-targeting guide RNA). Cells were harvested and analyzed by immunoblot. The levels of protein expression by p-IRF3 (S386 phosphorylated, active IRF3), IRF3 (total IRF), IFIT1, RIG-I, and MDA5 relative to the expression level of tubulin were determined using respective antibodies. (6B) HepG2-hNTCP-NT, RKO, and MKO cells were treated with 100 ng,/ml of X-RNA or 100 ng/ml or 200 ng/ml of poly-U/UC PAMP for 24 hr. Cells were harvested and analyzed by immunoblot as in (6A). (6C and 6D) Cells were infected with HBV at moi of 1000 Geq/cell. After 24 hour cultures were treated with DMSO (−; negative control), CsA (treatment control), (6C) 10 μM) or F7 or (6D) X RNA or poly-U/UC PAMP. Three days later the cells were harvested, Hirt extracts prepared and subjected to Southern blot analysis using as HBV-DNA probe. The positions of mass markers are indicated. Values beneath each lane show percent cccDNA remaining compared to control treatment.

FIGS. 7A-7C illustrate suppression of cccDNA in primary human hepatocytes. (7A) PHH were cultured alone or were treated with 100 ng/ml X-RNA (X100), or 50 ng/ml (P50), 100 ng/ml (P100) or 200 ng/ml (P200) poly-U/UC PAMP or were infected with SenV (control) and harvested 24 and 72 hours later. The cell lysates were analyzed by immunoblot for p-IRF3 (S386 phosphorylated, active IRF3), IRF3 (total IRF3), IFIT1, and Actin (control) using respective antibodies. (7B) PHH cultures were cultured alone or were treated with 100 ng/ml X-RNA (X100), or 100 ng/ml (P100) or 200 ng/ml (P200) poly-U/UC PAMP or were infected with SenV (control) and harvested 24 and 72 later. Cells were harvested, RNA extracted and analyzed by RT-qPCR to measure the expression levels of the indicated innate immune genes normalized to the level of GAPDH expression. Values are shown on the heat map as mean fold induction over nontreated cells from three independent experiments. (7C) PHH cultures were inoculated with HBV at moi of 200 Geq/cell. 24 hours later the cultures were treated with CsA (treatment control; 10 μM), 100 ng/ml X-RNA (X100), or 100 ng/ml (P100) or 200 ng/ml (P200) poly-U/UC PAMP. Three days later the cells were harvested, DNA isolated by isolated Hirt extraction, and analyzed by Southern blot using a HBV-specific probe. Viral protein-free DNAs (protein-free Relaxed Circular DNA [PF-RC DNA]and cccDNA) are noted. Values under each lane show the percent remaining cccDNA compared to nontreated-control. The positions of mass markers are shown at left.

FIGS. 8A-8L are a series of graphs illustrating that F7 and poly-U/UC induce differential innate immune genes expression. HepG2-hNTCP cells were infected with SenV (positive control) or treated with F7 (5 or 10 uM as indicated), X-RNA (100 ng/ml; X-100 and 200 ng/ml; X-200) or poly-U/UC PAMP 100 ng/ml or 200 ng/ml as indicated for 24 (black columns) and 72 hours (gray columns). Cells were harvested at each time point. Total cellular RNA was purified and subjected to RT-qPCR analysis to measure the expression level of a panel of innate immune genes including IFIT1, CXCL10, IFITM1, RSAD2, RIG-I, MDA5, SAMHD1, APOBEC3A, APOBEC3G, IFN-α, IFN-β, and IFN-λ3. Gene expression levels were normalized to the level of GAPDH expression in each sample and are expressed as the fold induction over that achieved with 2.5% DMSO treatment (negative control) from three independent experiments. Data was presented as mean±standard deviation (SD), *P<0.01, **P<0.005, ***P<0.0005, ****P<0.0001, and ns=non-significant.

FIGS. 9A-9E illustrate kinetics of HBV replication in parallel cultures of cells during treatment with F7 or poly-U/UC PAMP. (9A) HepG2-hNTCP cells were infected with HBV at a moi of 1000 Geq/cell. After 24 hours the cells were treated with F7 (10 μM) (upper), or 100 ng/ml X RNA or 100 ng/ml poly-U/UC PAMP (lower). Cells were harvested at each time point, Hirt supernatants prepared and subjected to Southern blot analysis. (B) HBV pgRNA was analyzed by RT-qPCR. Values from 20 Dpi of ‘HBV only’ was set to 100% for RT-PCR analysis. Data was presented as mean±standard deviations (SD), ***P<0.001, ****P 21 0.0001; and ns=non-significant (9C) HBV intracellular capsid-associated DNA from cells treated with 10 uM F7 (upper) or 100 ng/ml XRNA or poly-U/UC PAMP (lower) was analyzed by Southern blot. (9D) Secreted HBsAg from HBV-infected cells treated with F7 (upper) or poly-U/UC (lower) was detected by ELISA. (9E) Extracellular HBV-DNA was measured by qPCR from cells treated with 10 uM (left) or 100 ng/ml XRNA or poly-U/UC PAMP (right). Data are presented as mean±standard deviation (SD) from three independent experiments. ***P 0.0002.

FIGS. 10A-10E graphically illustrate CC₅₀, analysis of F7 and poly-U/UC PAMP treatment. HepG2-NTCP Cells (10A and 10C), dHepaRG (10B and 10D), and PHH (10E) were treated with increasing doses of F7 or poly-U/UC PAMP for 72 hours. Cell viability was determined concurrently by measuring ATP content, with values normalized to mock-treated cells. Graph represents the mean of triplicated samples in each of 3 independent experiments, with error bars showing standard deviation.ns=non-significant.

FIG. 11 illustrates the half-life of cccDNA, HepG2-NTCP cells were infected with HBV at an moi 1000 Geq/cell/ At 3 dpi the cultures were left nontreated or were treated with ETV (500 nM) through the full 50 day time course by replacing the media each day with fresh media alone or containing ETV. Cells were harvested at the indicated time points, DNA was isolated by Hirt extraction and analyzed by Southern blot using a HBV-specific probe. Percentage values below each lane indicate the relative amount of cccDNA present compared to day 3 levels.

FIG. 12 illustrates immunoblot analysis of HepG2-hNTCP cells transduced with CRISPR/Cas9 guide RNA constructs to target RIG-I (RKO) or MDA5 (MRO) or nontargeting guide RNA (NT) control. Cells were treated with 100 U/ml IFN-β for 24 hrs. Cell lysates were prepared and analyze by immunoblot. The levels of RIG-I, MDA5, and IFIT1 and tubulin (house-keeping protein control) were determined using respective antibodies.

### DETAILED DESCRIPTION

Hepatitis B virus (HBV) mediates persistent infection, chronic hepatitis, and liver disease. HBV covalently-closed-circular DNA (cccDNA) is central to viral persistence such that its elimination is considered the cornerstone for HBV cure. Inefficient detected by pathogen recognition receptors (PRRs) in the infected hepatocyte facilitates HBV persistence via avoidance of innate immune activation and interferon regulatory factor 3 (IRF3) induction of antiviral gene expression. In view of the foregoing challenges, the inventors evaluated induced signaling of RIG-I, a PRR that signals innate immunity, for ability to suppress cccDNA. As described in more detail below, two distinct RIG-I agonists, a small molecule compound, referred to as “F7”, and a 5′-triphosphate-poly -U/UC pathogen-associated-molecular-pattern (PAMP) RNA were employed for proof of concept. F7 and poly-U/UV PAMP treatment of HBV-infected cells induced RIG-I signaling of IRF3 activation to induce antiviral genes for suppression of cccDNA formation and accelerated decay of established cccDNA and were additive to the actions of Entecavir. This study demonstrates that activation IRF3, such as through induction of the RIG-I pathway, induces innate immune actions offers therapeutic benefit toward elimination of cccDNA.

In accordance with the foregoing, in one aspect the disclosure provides a method for suppressing hepatitis B virus (HBV) covalently-closed-circular DNA (cccDNA) levels in an infected cell. The method comprises contacting the infected cell with an agent that induces interferon regulatory factor 3 (IRF3) activation in the infected cell.

As indicated above, hepatitis B virus DNA is released from the viral nucleocapsid in the nucleus of the infected cell. This viral DNA is released from the viral capsid as relaxed circular DNA (RC DNA). The RC DNA is converted to covalently closed circular DNA (cccDNA) and serves as the main template for the synthesis of all HBV RNA transcripts including pregenomic (pg) RNA, pre-S, S, and X viral RNAs, cccDNA can be replicated within the cell through the intracellular amplification pathway. The cccDNA can be relatively long-lived and can be the basis for long-term, chronic infections, even after treatments. As used herein, the term “suppressing cccDNA”comprises inhibiting formation of new cccDNA in the infected cell. The term “suppressing cccDNA”can also encompass reducing the stability of existing cccDNA in the infected cell prior to the contacting step. The reduction in stability can lead to a decreased half-life of the cccDNA. The reduced stability can be observed by a reduction in levels of cccDNA within the infected cell. In some embodiments, the reduction of levels of cccDNA is relative to an infected cell (e.g., of the same lineage or tissue type) that is also infected with HBV. The reduction can be any detectable reduction, such as about 5% reduction, about 10% reduction, about 15% reduction, about 20% reduction, about 25% reduction, about 30% reduction, about 35% reduction, about 40% reduction; about 45% reduction, about 50% reduction, about 55% reduction, about 60% reduction, about 65% reduction, about 70% reduction, about 75% reduction, about 80% reduction, about 85% reduction, about 90% reduction, about 95% reduction, about 97% reduction, about 99% reduction, and total eradication of cccDNA levels in the infected cell.

The cell can be any cell that is infected with HBV. In some embodiments, the infected cell is a hepatocyte.

A key component of the innate immune response against viral infections is the activation of interferon regulatory factor 3 (IRF3). IRF3 induces the expression of antiviral genes and also induces IFN. The antiviral genes can suppress virus replication in the infected cell while IFN directs the suppression of virus replication both in the infected cell and neighboring bystander cells through the expression of hundreds of interferon stimulated genes (ISGs) that have antiviral and immune-modulatory activities. In addition to IFN suppression of viral infections, programed death of virus-infected cells can serve to prevent virus spread. Thus, the combination of IRF3 actions, including resulting IFN actions and cell death signaling, impart a synergistic program of virus control for many viruses. The present disclosure is based in part on a demonstration that this pathway can be leveraged against HBC, which typically avoids inducing such IRF3 actions.

In some embodiments, the agent that induces IRF3 activation indirectly by inducing a retinoic acid-inducible gene I (RIG I) like receptor (RLR) signaling pathway, The RLRs are cytoplasmic RNA helicases that function as PRRs for the recognition of RNA virus infection. The RLRs include RIG-I (retinoic acid-inducible gene I), MDA5 (melanoma differentiation-associated gene 5), and LGP2 (laboratory of genetics and physiology 2). Whereas RIG-I and MDA5 encode tandem amino-terminal caspase activation and recruitment domains (CARDs), LGP2 lacks CARDs and is thought to play a regulatory role in signaling initiated by RIG-I or MDA5. Following the recognition and binding of viral PAMP RNA, RIG-I signals through the adaptor protein mitochondrial antiviral signaling (MAVS; also known as IPS-1/VISA/Cardif), Downstream signaling by the RLRs induces the activation of latent transcription factors, including interferon regulatory factor (IRF)-3 and NF-KB, leading to the production of type-I interferons (IFN) from the infected cell. Persons of ordinary skill in the art would readily be able to determine the activation of an RLR pathway, such as by assaying the transcription of known downstream RLR-regulated genes. For example, in some embodiments, RLR activation can be established by an increase in or IFN-β or ISG54 expression. In another embodiment, RLR activation can be established by an increase in IRF-3 phosphorylation. Accordingly, in some embodiments, the RLR signaling pathway comprises RIG I, melanoma differentiation associated gene 5 (MDA5), laboratory of genetics and physiology 2 (LGP2), and/or mitochondrial antiviral signaling (MAVS) protein.

In some embodiments, the agent inducing RIG-I signaling is or comprises a nucleic acid molecule comprising a pathogen-associated molecular pattern (PAMP). Exemplary PAMPs and PAMP-containing nucleic acid molecules encompassed by the present disclosure are disclosed in U.S. Pub. Nos. 2015/0017207 and 2018/0104325, which address PAMP induction of innate immune response signaling and are incorporated herein by reference in their entireties. Elements and exemplary embodiments of the PAMP containing nucleic acid encompassed by the disclosure are addressed here.

As a preliminary matter, used herein, the term “nucleic acid”refers to a polymer of monomer units or “residues”. The monomer subunits, or residues, of the nucleic acids each contain a nitrogenous base (i.e., nucleobase) a live-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy -methylcytosine, 8-oxoguanine, 2-amino-adenosine, 2-amino-deoxyadenosine, 2-thiothymidine, pyrrolopyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

The five-carbon sugar to which the nucleobases are attached can vary depending on the type of nucleic acid. For example, the sugar is deoxyribose in DNA and is ribose in RNA. In some instances, herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo”or deoxyrobo” prefix before the nucleobase to infer the type of five-carbon sugar. For example, “ribocytosine”as occasionally used herein is equivalent to a cytidine residue because it indicates the presence of a ribose sugar in the RNA molecule at that residue. The nucleic acid polymer can be or comprise a deoxyribonucleotide (DNA) polymer, a ribonucleotide (RNA) polymer, including mRNA. The nucleic acids can also be or comprise a PNA polymer, or a combination of any of the polymer types described herein (e.g., contain residues with different sugars).

In some embodiments, the PAMP-containing nucleic acid is synthetic. In this context, the term “synthetic”refers to non-natural character of the nucleic acid. Such nucleic acids can be synthesized de novo using standard synthesis techniques. Alternatively, the nucleic acid PAMPs can be generated or derived from naturally occurring pathogen sequences using recombinant technologies, which are well-known in the art. In some embodiments, the sequence of the synthetic nucleic acid PAMP construct is not naturally occurring.

In some embodiments, the PAMP-containing nucleic acid is an RNA construct. In some of these embodiments, the PAMP-containing nucleic acid is derived from, or reflects the sequence of, the HCV poly-U/UC region and, in this context, may be generally referred, to as the poly-U/UC PAMP RNA construct. In some embodiments, the poly-U/UC PAMP RNA construct is synthetic.

The PAMP-containing nucleic acid of this disclosure generally comprises (a) a 5′-arm region comprising a terminal triphosphate (“ppp”or “3×p”); (b) a poly-uracil core (also referred to as a “poly-U core”); and (c) a 3′-arm region. In one embodiment, the three regions (a, b, and c) are covalently linked in a single nucleic acid polymer macromolecule. The covalent linkage can be direct (without interspersed linker sequence(s)) or indirect (with interspersed linker(s) and/of sequences(s)). In one embodiment, the 5′-arm region is covalently linked to the 5′-end of the poly-U core. In one embodiment, the 3′-arm region is covalently linked to the 3′-arm region of the poly-U core. The polymer can be single or double stranded or can appear with a combination of single and double stranded portions.

In one embodiment, the poly-U core comprises at least 8 contiguous uracil residues. In further embodiments, the comprises between 8 and 60 contiguous uracil residues, such as 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 and contiguous uracil residues. In one embodiment, the poly-U core comprises more than 8 contiguous uracil residues. In one embodiment, the poly-U core comprises 12 or more contiguous uracil residues. In some embodiments, the poly-U core consists of a plurality of contiguous uracil residues, such as 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 contiguous uracil residues.

In one embodiment, the 3′-arm region comprises a 5′-most nucleic acid residue that is not a uracil residue. Instead, the 5′-most nucleic acid residue of the 3′-arm region can be an adenine, guanine, or cytosine residue, or any non-canonical residue. In one embodiment, the 5′-most nucleic acid residue of the 3′-arm region is a cytosine residue, a guanine residue, or an adenine residue.

In one embodiment, the nucleotide composition of the 3′-arm region is at least about 40% uracil residues. In some embodiments, the 3′-arm region is at least about 45%, is at least about 50%, is at least about 60%, is at least about 70%, is at least about 80%, is at least about 90%, or is at least about 95 uracil residues. In one embodiment, the 3′-arm region comprises a plurality of short stretches (for example, between about 2 and about 15 nucleotides in length) of contiguous uracil residues with one or more cytosine residues interspersed therebetween. In one embodiment, the 3′-arm region comprises a plurality of short stretches (for example, between about 2 and about 15 nucleotides in length) of contiguous uracil residues with one or more guanine residues interspersed therebetween. In one embodiment, the 3′-arm region comprises a plurality of short stretches (for example, between about 2 and about 15 nucleotides in length) of contiguous uracil residues with one or more adenine residues interspersed therebetween. In one embodiment, the 3′-arm region comprises a stretch of consecutive uracil residues that does not exceed the length of the poly-U core of the synthetic PAMP-containing nucleic acid molecule. In one embodiment, the 3′-arm region does not comprise a stretch of consecutive uracil residues that equals and/or exceeds the length of the poly-U core of the synthetic PAMP-containing nucleic acid molecule. In some embodiments, the 3′-arm region comprises at least 7 consecutive uracil residues, such as 7, 8, 9, 10, 11, 12, 13, 14, 15, 16, 17, 18, 19, 20, 21, 22, 23, 24, 25, 26, 27, 28, or 29, contiguous uracil residues.

At a minimum, the 5′-arm region consists of a terminal tri-phosphate (ppp) moiety. In such embodiment, the triphosphate is at the 5′-terminus of the synthetic PAMP-containing nucleic acid molecule and can be represented as “5′-ppp”. In a further embodiment, the terminal triphosphate is linked directly to the 5′-end of the poly-U core sequence. In an alternative embodiment, the 5′-arm region comprises the 5′-end terminal triphosphate and one or more additional nucleic acid residues, the sequence of which terminates with a 3′-end. The one or more additional nucleic acid residues in the 5′-arm region of this embodiment are disposed between the terminal triphosphate and the 5′-most uracil residue of the poly-U core. Persons of ordinary skill in the art will readily appreciate that the one or more additional nucleic acid residues in the 5′-arm region can be any number of nucleic acid residues and can present any sequence without limitation. The sequence of the one or more additional nucleic acid residues in the 5′-arm region does not affect the functionality of the PAMP-containing nucleic acid molecule. For instance, as described in U.S. Pub. Nos. 2015/0017207 and 2018/0104325, the addition of a poly-U core region to a non-stimulatory nucleic acid that contains a 5′-triphosphate (such as the HCV X region) confers stimulator properties for innate immune system signaling. In one embodiment, the sequence of the one or more additional nucleic acid residues in the 5′-arm region does not consist of the entire 5′-end portion of a naturally occurring HCV genome sequence that naturally occurs “upstream”or 5′ to the poly-U core of the poly-U/UC region for that HCV strain. Stated differently, in this embodiment the entire synthetic PAMP-containing nucleic acid molecule is not a naturally occurring HCV genome, complete with the 5′ triphosphate, the entire coding region, and the untranslated 3′ poly-U/UC region. Accordingly, in this embodiment, the 5′-arm region, the one or more nucleic acid residues of the 5′-arm region, and the pole-uracil core do not naturally occur together in an HCV genome. However, in this embodiment, the one or more nucleic acid residues of the 5′-arm region can comprise or consist of a subfragment of the entire naturally occurring sequence that exists between the 5′-arm region and the poly-uracil core. Alternatively, in this embodiment, the one or more nucleic acid residues of the 5′-arm region can comprise sequence in addition to a portion or the entire naturally occurring HCV genome sequence that exists between the 5′-end and the poly-uracil core.

In some embodiments, the nucleic acid molecule comprises a sequence of at least 16 nucleotides. In some embodiments, the nucleic acid molecule comprises a sequence of at least about 16 nucleotides to about 1000 nucleotides, such as between about 20 and about 1000 nucleotides, between about 30 and about 1000 nucleotides, between about 40 and about 1000 nucleotides, between about 50 and about 1000 nucleotides, between about 60 and about 1000 nucleotides, between about 70 and about 1000 nucleotides, between about 80 and about 1000 nucleotides, between about 90 and about 1000 nucleotides. between about 100 and about 1000 nucleotides, between about 150 and about 1000 nucleotides, between about 200 and about 1000 nucleotides, between about 250 and about 1000 nucleotides, between about 300 and about 1000 nucleotides, between about 350 and about 1000 nucleotides, between about 400 and about 1000 nucleotides, between about 450 and about 1000 nucleotides, between about 500 and about 1000 nucleotides, between about 550 and about 1000 nucleotides, between about 600 and about 1000 nucleotides, between about 650 and about 1000 nucleotides, and between about 700 and about 1000 nucleotides, and any number or range therein. In yet further embodiments, the nucleic acid contains between about 20 and about 100 nucleotides, between about 30 and about 100 nucleotides, between about 40 and about 100 nucleotides, between about 50 and about 100 nucleotides, between about 60 and about 100 nucleotides, between about 70 and about 100 nucleotides, as between about 80 and about 100 nucleotides, and between about 90 and about 100 nucleotides, and any number or range therein. In some embodiments, the nucleic acid comprises between about 16 and 60 nucleotides, such as between about 16 and about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 nucleotides. In some embodiments, the nucleic acid has up to about 50, 51, 52, 53, 54, 55, 56, 57, 58, 59, and 60 nucleotides and the 3′-arm comprises a plurality of short stretches between 2 and 15 contiguous uracil residues with one or more cytosine or guanine residues interspersed between the plurality of short stretches. In some embodiments, the nucleic acid molecule has up to 53 nucleotides and the 3′-arm region is at least 60% uracil residues

Non-limiting examples of nucleic acid sequences of PAMPs encompassed by the disclosed PAMP-containing nucleic acids containing are disclosed in U.S. Pub. Nos. 2015/0017207 and 2018/0104325 and Saito, T., et al. (2008). Innate immunity induced by composition-dependent RIG-I recognition of hepatitis C virus RNA Nature 454, 523-527; and Schnell, G., et al. (2012). Uridine composition of the poly-U/UC tract of HCV RNA defines non-self recognition by RIG-I. PLoS pathogens 8, e1002839 (each of which is incorporated herein by reference) and are set forth herein as SEQ ID NOS:34-123. In some embodiments, the PAMP-containing nucleic acids contains poly-U core region and/or 3′-arm sequences independently selected from any of the poly-U core region and/or 3′-arm regions in any of the disclosed sequences of SEQ ID NOS:34-123. These exemplary, non-limiting sequences are also provided below in TABLE 1. The disclosed PAMP-containing nucleic acids can comprise any of the sequences listed therein. It will be understood that such exemplary PAMP containing nucleic acids would comply with the general structural parameters of the PAMP containing molecules, as described herein, including having a terminal tri-phosphate (ppp) moiety. In one embodiment, the PAMP-containing molecule comprises the sequence: GGCCAUCCUGUUUUUUUCCCUUUUUUUUUUUUCUCCUUUUUUUUUCCUCUUU UUUUCCUUUUCUUUCCUUU (SEQ ID NO:124). In another embodiment, the PAMP-containing nucleic acid comprises the sequence: GGCCAUUUUCUUUUUUUUUUCUCUUUUUUUUUUUUUUUUUUAUUUUCUUU AAU (SEQ ID NO:125). Again, it will be understood that the exemplary PAMP-containing nucleic acid with the indicated sequences will also possess the characteristics described above, including a 5′ terminal tri-phosphate (ppp) moiety.

TABLE 1
sequences of polyU/UC PAMP constructs with exemplary 5′-arm, ploy U core,
and 3′-arm domains

| | | | SEQ ID |
|---|---|---|---|
| 5′ arm^{a} | U-core | 3′ arm | NO: |
| GGCCAUCCUGUUUUUUUCC | U34 | CUCCUUUUUUUUUCCUCUUUUUUUCCUUUUCUUU | 34 |
| C(U11)C | | CCUUU | |
| ACUGUUCC | U43 | C(U14)CCCUCUUUCUUCCCUUCUCAUCUUAUUCUA | 35 |
| | | CUUUCUUUCUU | |
| GGCCAUCCUGUUUUUUUCC | U34 | CUCCUUUUUUUUUCCUCUUUUUUUCCUUUUCUUU | 36 |
| C(U11)C | | CCUUU(C26) | |
| GGCCAUCCUGUUUUUUUCC | U34 | CUCCUUUUUUUUUCCCCCCCCCCCCCCCCCCCCCC | 37 |
| C(U11)C | | CCCC | |
| GGCCAUCCUGUUUUUUUCC | U36 | CCUUUUUUUUUCCUCUUUUUUUCCUUUUCUUUCC | 38 |
| C(U11)C | | UUU | |
| UUUUUUUUUUUUUUUUUU | U34 | VUUUUUUUUUUUUUUUUUUUUUUUUUUUUUUUU | 39 |
| UC(U11)C | | UUUUUU | |
| GGCCAUCCUGUUUUUUUCC | U9 | CCUCUUUUUUUCCUUUUCUUUCCUUU(C26) | 40 |
| C(U11)C(U8)UCC | | | |
| GGCAUCCUGUUUUUUUCC | U17 | CUCCUUUUUUUUUCCUCUUUUUUUCCUUUUCUUU | 41 |
| C(U11)C | | CCUUU(C17) | |
| GGCCAUCCUG | U34 | CUCCUUUUUUUUUCCUCU | 42 |
| CCCCCCCCCC | U34 | CUCCUUUUUUUUUCCUCU | 43 |
| GGCCAUCCUG | U4 | CCCCCCCCCCCCCCCCCC | 44 |
| | U44 | VUUUUUUUUUUUUUUUUU | 45 |
| | U44 | CUUUUUUUUUUUUUUUUU | 46 |
| UUCCUUCC | U36 | CUCCUUUUUUUUUCCUCU | 47 |
| UUUUUUUUUG | U34 | CUCCUUUUUUUUUCCUCU | 48 |
| UUUUUUUUUG | U34 | CUUUUUUUUUUUUCCUUU | 49 |
| GGUUUUCC | U36 | CUUUUUUUUUUUUUUUUU | 50 |
| GGCCAUCCUG | U10 | C(U10)C(U10)CUCUCCUUUUUUUUUCCUCU | 51 |
| GGCCAUCCUG | U15 | C(U15)CUUCUCCUUUUUUUUUCCUCU | 52 |
| GGCCAUCCUG | U10 | CCC(U10)CCC(U8)CUCCUUUUUUUUUCCUCU | 53 |
| GGCCAUCCUG | U18 | CCCCCC(U10)CUCCUUUUUUUUUCCUCU | 54 |
| GGCCAUCCUGUUUUUUUCC | U34 | CUCCUUUUUUUUUCCUCUUUUUUUCCUUUUCUUU | 55 |
| C(U11)C | | CCUUU | |
| GGCCAUUC | U16 | CUUUCUUCUUU | 56 |
| GGCCAUUCCC | U81 | CUCCUUCUUUUCUUUAUUCCUUCUUU | 57 |
| GGCCGUUCC | U64 | CUUUUCCCCUUUUUUAUUUUUCUUUCUU | 58 |
| GGCCAUCCUGUUUUUUUGU | U43 | CUUUUUUUCCCUUUUUUUUAUUUUAUUUUCUUU | 59 |
| UUUUUC | | UGGU | |
| GGCCAUCCCCC | U96 | CCUCUUUUUUUCCUUUUCUUCUUU | 60 |
| GGCCGUUCUG | U85 | CCUUUUUUUUAUUCCUCUUCU | 61 |
| GGCCAUCCCCUUUG | U94 | AUUUCUCCUUCUUUU | 62 |
| GGCCAUUCC | U25 | CUUUUUUUUUCC(U24)CCUUUUCUUUCUUCUUU | 63 |
| GGCCAUUUUC | U14 | CUUCUUUCUUUUUCUUUUUCUUUUUUUCCUUCUU | 64 |
| | | U | |
| AGCCAUUUCCUG | U28 | CUUUUUUUUUUUCUUUCCUUUCCUUCUUUUUUUC | 65 |
| | | CUUUCUUUUUCCCUUCUUUAAU | |
| GGCCAUUUCCUG(U15)GG | U39 | CCUUUCCUUCUUUUUUUUUUUUUCCCUCUUUAU | 66 |
| GGCCAUUUCCUG | U34 | CUUUUCCUUCUUUUUCCCUUUUUCUUUCUUCCUU | 67 |
| | | CUUUAAU | |
| GGCCAUCCUGUG | U75 | AUUUCCUUUUCUU | 68 |
| 5′GGCCAACCUG(U26)CC | U34 | CCUUUUUUUCUUUUUUUUUUUUUUUUUCCUUCCU | 69 |
| | | UUU | |
| GGCCAUCCUG | U16 | CUUUCUUU | 70 |
| GGCCAUUUUUCC | U23 | CUUUUUUUUUUCCUUUUUUUCUUUUUUUUUCUU | 71 |
| | | UUCUUU | |
| GGCCAUUC | U35 | CGUUUCUUUUUCUUCUUUUUGUUUUCUCUUCUCC | 72 |
| | | UUUU | |
| GGCCAUUCCCC(U14)CCGC | U33 | CUUUUUUUUUCC*U27)CUUUUU | 73 |
| GGCCAUCCCCC(U13)CCGC | U21 | CUUUUUUUUUUUCUUUUUUUUUUCC(U24)CUUUU | 74 |
| | | CUUUUU | |
| GGCCAUUC | U16 | CUUUCUUCUUU | 75 |
| GGCCAUCCCCUUC | U22 | CCUUUUCUUCUUU | 76 |
| GGCCAUCCUGUUUUUUUCC | U29 | CUCCUUUUUUUUUCCUCUUUUUUUCCUUUUCUUU | 77 |
| C(U11)C | | CCUUU | |
| GGCCGUCCUG(U19)CC | U67 | CUUCUUUCUUUCUU | 78 |
| GGCCAUUUCCUG | U53 | C(U17)CC(U20)CUUUCCUUCUUUUUUCCUUUCUUU | 79 |
| | | UCCUUCCUUCUUUAAU | |
| GGCCAUUCCUG(U16)CUUU | U17 | CCUUUC(U15)CCUUUCUUCUUUAAU | 80 |
| UGUUUUUUUUG | | | |
| GGCCAUUUCCUG | U20 | CUUUCCUUCUUUUUUCCUUUCUUUUCCUUCCUUC | 81 |
| | | UUUAAU | |
| GGCCAUUUCCUG | U34 | CUUUUCCUUCUUUUUCCCUUUUUCUUUCUUCCUU | 82 |
| | | CUUUAAU | |
| GGCCAUUUCCUG | U51 | C(U17)CC(U20)CUUUCCUUCUUUUUUCCUUUCUUU | 83 |
| | | UCCUUCCUUCUUUAAU | |
| GGCCAUUUCCUG(U14)CCC | U37 | CUUUCCUUCUUUUUUUUCCUUUCUUUUCCUUCCU | 84 |
| | | UCUUUAAU | |
| GGCAUCCUG | U65 | CUUUUCUUU | 85 |
| ACACUCCAUUUCUUUUUUU | U67 | CUUUUUCUUUCCUUUCUUUUCUGACUUCUAAUUU | 86 |
| G | | UCCUUCUUA | |
| GUCCUUCUG | U78 | CCUUACCCUUUCCUUCUUUUCUUCCUUUUUUUUC | 87 |
| | | CUUACUUU | |
| GGGUCCCCUUG | U12 | CUUUCCUUCUUUCCUUUCCUAAUCUUUCUUUCUU | 88 |
| AGCCAUUUCCUG | U28 | CUUUUUUUUUUUCUUUCCUUUCCUUCUUUUUUC | 89 |
| | | CUUUCUUUUUCCCUUCUUUAU | |
| GGCCAUUUCCUG(U15) | U55 | CCUUUCCUUUUUUUUUUUUUUUCCCUUUUUAU | 90 |
| GGCCAUCCUG(U22)C | U17 | CUUUUUUUUUCUUCUUUUUCUUUCC(U24)CUUCU | 91 |
| | | UUC | |
| GGCCAUUUCCUG | U46 | CUUUUUCCCUCUUUUUCUUCUCUUUUUCCUUCUU | 92 |
| | | UAAU | |
| GCUAACUGUUCC | U43 | CUUUUUUUUUUUUUUCCCUCUUUCUUCCCUUCUC | 93 |
| | | AUCUUAUUCUACUUUCUUUCUU | |
| GCUAACUGUUCC | U38 | C(U15)CCCUCUUUCUUCCCUUCUCAUCUUAUUCUA | 94 |
| | | CUUUCUUUCUU | |
| GCUAACUGUUCC(U11)C | U27 | CCUUCUUUCUUUCUUUCUUACCUUACUUUACUUU | 95 |
| | | CUUUUCU | |
| GCUAACAGUUUCUC(U13)CC | U25 | AUUUUCUUUUCCUUUCUUUCUCACCUUACAUUAC | 96 |
| (U6)AUUUUUA | | UUUCUUUCUU | |
| GCUAAUUUCCUUAUUG | U19 | CUUUCCAUUUCCUUCCUUCUUACUUCACUUUACC | 97 |
| | | UUCUUUCU | |
| GCUAACUG | U77 | CCUUUCCUUUCUUUCUUACCUUACUUUACAUUCU | 98 |
| | | UUUCU | |
| GCUAACUGUUCC | U70 | CUUUCCUUCCUUUCUCACCUUCUUUUACUUCUUU | 99 |
| | | CCU | |
| GCUAACUG | U45 | CUUUUCUUUCCUUUCCUUCUUACUCUACUUUACU | 100 |
| | | UUUUCU | |
| GCUAACUGUUC | U78 | CUUUUCCUUCUUCUUUCUUACCUUAUUUUCCUUC | 101 |
| | | UUUCUU | |
| GCUAACUG | U30 | CUUUUUUUUUCUUUUCUUUCCUUCUUACCUUACU | 102 |
| | | UUACUUUCUUUUCU | |
| GCUAACUG | U81 | CCUUUUUCCUUUUCCUUCUCUUUUUACCUUACUU | 103 |
| | | UACUUUUCUU | |
| GCUAACUGUCCC | U84 | CUUUUUUUCUCUUUUCCUUCUUUCUUACCUUAUU | 104 |
| | | UUACUUUCUUUCCU | |
| GCUAACUGUCCCUUUUUUU | U30 | C(U18)GUUUCUUUUCCUUCUCAUUUCCUUCUUAUC | 105 |
| UUG | | UUAAUUACUUCCUUUCCU | |
| GCUAACUG | U39 | CCUUCUUCCUUUCCUUCUUACCUUACUUUAUUUU | 106 |
| | | CUUUCCU | |
| GCUAACUG | U54 | CUUUCUUUUCUUUUCUCACCUUACUUUACUUCCU | 107 |
| | | UUCUU | |
| GCUAGUUUUC | U24 | G(U14)CCUCUUUUUCCGUAUUUUUUUUUUUUCCU | 108 |
| | | CUUUUCUU | |
| GGCCAUCCUG(U7)CCC(U11) | U34 | CUCC(U9)CCUC(U7)CCUUUUCUUUCCUUU | 109 |
| C | | | |
| CCAUUUUUC | U13 | GUUUG(U16)CUUUCCUUCUUUCCUGACUUUUAAU | 110 |
| | | UUUCCUUCUUA | |
| CCAUUUUUC | U49 | GUUUG(U17)CUUUCCUUCUUUCCUGACUUUUAAU | 111 |
| | | UUUCCUUCUUA | |
| GGCCAUUUCCUG | U33 | ACCCUUUUUUCUC(U12)CCUUCUUCUUUAAU | 112 |
| GGCCAUUUCCUG | U18 | ACCCUUUUUUCUC(U17)CCUUCUUCUUUAAU | 113 |
| GGCCAUUUUCUG | U14 | AUUUUCUUUAAU | 114 |
| GGCCAUUUUC(U10)CUC | U18 | AUUUUCUUUAAU | 115 |
| GGCCAUUUUCUG | U20 | CC(U12)CCUC(U20)AUUUUCUUUAAU | 116 |
| GGCCAUUUUCUG(U12)C | U17 | CCUUUUUUUUUCUC(U14)AUUUUCUUUAAU | 117 |
| GGCCAUCCUG | U24 | G(U17)CUUUUUCC(U13)AUUUUCUUCUUU | 118 |
| GGUCCUAAG | U13 | CUUCCUUCCUUCUUUCCUUUUCUAAUUUUCCUUC | 119 |
| | | UUU | |
| GGUCCUAAGUUG | U15 | CCUUCCUUCUUUCCCUUUUCUAAUUUUCCUUCUU | 120 |
| | | U | |
| GGUCCUAAGUUG | U23 | CCUUUCCUUCCUUCUUUCCUUUUCUAAUUUUCCU | 121 |
| | | UCUUU | |
| GGCCAUUUCUG | U41 | GUUUCCUUCUUUUUCCUUUUC(U11)CUCCCUUUAA | 122 |
| | | U | |
| GGCCAUUUCUG | U14 | GUUUCCUUCUUUUUCCUUUUC(U13)CUCCCUUUAA | 123 |
| | | U | |

^{a}The 5′ arms all contain 5′-ppp moiety in addition to the indicated sequence.

As described in U.S. Pub. Nos. 2015/0017207 and 2018/0104325, nucleic acids with HCV-derived RNA PAMPs having poly-uracil core sequences can trigger retinoic acid-inducible gene I (RIG-I)-like receptor (RLR) signaling. Thus, in some embodiments, the PAMP-containing nucleic acid molecule is capable of inducing retinoic acid-inducible gene I (RIG-I)-like receptor (RLR) activation. In one embodiment, the RLR is RIG-I. Persons of ordinary skill in the art would readily be able to determine the activation of an RLR, such as by assaying the transcription of known downstream RLR-regulated genes, as described in more detail below. For example, in some embodiments RLR activation can be established by an increase in IFN-β or ISG54 expression. In another embodiment, RLR activation can be established by an increase in IRF-3 phosphorylation.

In some embodiments, the PAMP-containing nucleic acid molecule is contacted to the cell at a concentration of about 80 ng/mL or greater to result in induction of RLR activation. Accordingly, in some embodiments, the method comprises contacting the cell with the PAMP-containing nucleic acid molecule agent at a concentration of least about 80 ng/mL to about 500 ng/mL, such as between 100 ng/mL and 250 ng/mL. In some embodiments, the method comprises contacting the cell with the PAMP-containing nucleic acid molecule at a concentration of least about 80 ng/mL, about 90 ng/mL, about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, about 200 ng/mL, about 210 ng/mL, about 220 ng/mL, about 230 ng/mL, about 240 ng/mL, about 250 ng/mL, about 260 ng/mL about 270 ng/mL, about 280 ng/mL, about 290 ng/mL, about 300 ng/mL, about 310 ng/mL, about 320 ng/mL, about 330 ng/mL, about 340 ng/mL/mL, about 350 ng/mL, about 360 ng/mL, about 370 ng/mL, about 380 ng/mL, about 390 ng/mL/mL, about 400 ng/mL, about 410 ng/mL, about 420 ng/mL, about 430 ng/mL, about 440 ng/mL, about 450 ng/mL, about 460 ng/mL, about 470 ng/mL, about 480 ng/mL, about 490 ng/mL, and about 500 ng/mL.

In some embodiments, the agent is or comprises a small molecule agent. Persons of ordinary skill in the art can readily identify small molecule agents that induce the RLR and/or IRF3 signaling pathways. Exemplary small molecule agonists that induce the RLR signaling pathway and/or IRF3 signaling pathway encompassed by the present disclosure are described in, e.g., Bedard, K. M., et al. (2012). Isoflavone agonists of IRF-3 dependent signaling have antiviral activity against RNA viruses. Journal of virology 86, 7334-7344; Pattabhi, S., et al. (2016). Targeting innate Immunity for Antiviral Therapy through Small Molecule Agonists of the RLR Pathway. Journal of virology 90, 2372-2387; Probst, P., et al. (2017). A small-molecule IRF3 agonist functions as an influenza vaccine adjuvant by modulating the antiviral immune response. Vaccine 35, 1964-1971, incorporated herein by reference in their entireties. In some embodiments, the small molecule agent is or comprises a benzothiazol-derivative molecule. Exemplary molecules are disclosed U.S. Pat. No. 9,884,876. In one particular embodiment, which was used as proof of concept in the studies described below, the small molecule agent has the chemical formula N-(6-benzamido-1,3-benzothiazol-2-yl)naphthalene-2-carboxamide. This small molecule is referred to herein as “F7”, and has the structure:

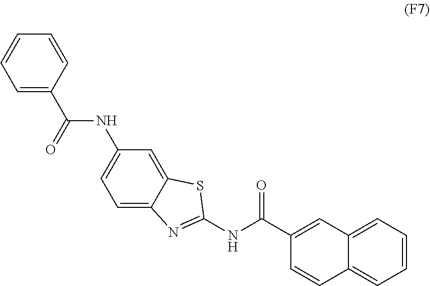 [SMILES: O=C(Nc1ccc2nc(NC(=O)c3ccc4ccccc4c3)sc2c1)c1ccccc1]

In some embodiments, the method comprises contacting the infected cell with two or more agents that induce IRF3 activation in the infected cell. The two or more agents (e.g., a first agent, a second agent, a third agent, etc.) can be contacted to the cell together, such as when formulated in a single admixture, or in separate administrations coordinated such that the effects of each agent is exhibited in the cell in overlapping time-frames. The two or more agents can comprise, for example, a PAMP containing nucleic acid and a small molecule agent. Each of the PAMP containing nucleic acid and the small molecule agent can encompass the features and embodiments of each agent as described above in more detail. For example, in one illustrative embodiment, the two or more agents comprise a nucleic acid comprising: a pathogen-associated molecular pattern (PAMP), wherein the PAMP comprises: a 5′ arm region comprising a terminal triphosphate: a poly uracil core comprising at least 8 contiguous uracil residues; and, a 3′ arm region comprising at least 8 nucleic acid residues, wherein the 5′ most nucleic acid residue of the 3′ arm region is not a uracil and wherein the 3′ arm region is at least 30% uracil residues. Additionally, the small molecule agent in this illustrative embodiment is or comprises a henzothiazol-derivative molecule, such as a small molecule comprising the chemical formula N-(6-benzamido-1,3-benzothiazol-2-yl)naphthalene-2-carboxamide.

In some embodiments, the method further comprises contacting the cell with a reverse transcriptase inhibitor in addition to the at least one agent that induces IRF3 activation in the infected cell, as described above. Exemplary reverse transcriptase inhibitors can include nucleotide or nucleoside reverse transcriptase inhibitors (NTRIs), which are analogs of naturally occurring nucleotides or nucleosides that are needed to synthesize viral DNA. The NTRIs compete with the natural deoxynucleotides for incorporation into the growing viral DNA. However, due to structural differences (e.g., a lack of a 3′ hydroxyl group), the chain extension is prevented because the next incoming nucleotide cannot form a phosphodiester bond needed to extend the chain. Exemplary, non-limiting NTRIs encompassed by the disclosure include Lamivudine, Adefovir dipivoxil, Entecavir, Telbivudine, Tenofovir, Tenofovir alafenamide (TAF), Clevudine, Besivo, Zadaxin, Remdesivir, and the like. Persons of ordinary skill in the art can select other appropriate reverse transcriptase inhibitors for performance of the disclosed methods.

In a specific embodiment, the method comprises contacting the cell with two or more of the following:

(1) a nucleic acid molecule comprising a pathogen-associated molecular pattern (PAMP), wherein the PAMP comprises:

a 5′ arm region comprising a terminal triphosphate;

a poly uracil core comprising at least 8 contiguous uracil residues; and

a 3′ arm region comprising at least 8 nucleic acid residues, wherein the 5′ most nucleic acid residue of the 3′ arm region is not a uracil and wherein the 3° arm region is at least 30% uracil residues, as described in more detail above;

(2) a small molecule agent, such as a benzothiazol-derivative molecule, such as N-(6-benzamido-1,3-benzothiazol-2-yl)naphthene-2-carboxamide; and

(3) an NRTI, such as selected from Lamivudine, Adefovir dipivoxil, Entecavir, Tedbivudine, Tenofovir, Tenofovir afenamide (TAF), Clevudine, Besivo, Zadaxin, Remdesivir, and the like.

In a specific embodiment, the method comprises contacting the infected cell with a nucleic acid molecule comprising a pathogen-associated molecular pattern (PAMP), as described above, and an NRTI, such as selected from Lamivudine, Adefovir dipivoxil, Entecavir, Telbivudine, Tenofovir, Tenofovir alafenamide (TAF), Clevudine, Besivo, Zadaxin, Remdesivir, and the like. As indicated above, the plurality of agents (including the NRTI), can be formulated in a combination or admixture, or can be contacted separately but in a coordinated fashion such that each exerts its effects in the cell in overlapping timelines. As described in more detail below, the simultaneous administration of the described agents results in synergistic effects on the suppression of cccDNA in the infected cells.

The method described above can be an in vitro method, applied to infected cell maintained in culture. In such embodiments, the method an include screening potential anti-viral agents for additional contribution to suppressing cccDNA.

Alternatively, the method can be an in vivo method performed with a subject with an HBC infection, or suspected of having an HBV infection, or is at risk of having an HBV infection. Accordingly, in another aspect, the disclosure provides a method of treating or preventing a hepatitis B virus (HBV) infection in a subject in need thereof. The method comprises administering to the subject a therapeutically effective amount of composition that induces interferon regulatory factor 3 (IRF3) activation in infected cells of the subject.

As used herein, the term “treat”refers to medical management of a disease, disorder, Or condition (e.g., HBV infection, as described above) of a subject (e.g., a human or non-human mammal, such as another primate, horse, dog, mouse, rat, guinea pig, rabbit, and the like). Treatment can encompass any indicia of success in the treatment or amelioration of a disease or condition (e.g., HBV infection). In this context; the term “”refers to preventing or suppressing the infection of colonization of a pathogen (e.g., hepatitis B virus). Additionally, the term “treating”refers to a therapeutic use, such as addressing an infection that has already started. In one embodiment, the term “treating”refers to curing the infection to a point where no active pathogens. (e.g., hepatitis B virus) remain in the host. In another embodiment, the term “treating”also encompasses slowing or inhibiting the spread of the infection within the body, such as slowing or inhibiting the replication rate of the pathogen (e.g., hepatitis B virus). The term also encompasses reducing the pathogenic burden in a cell (or host tissue or body). In some embodiments, this encompasses reducing the cccDNA levels in cells of the body. The term also encompasses accelerating the rate of clearance of the pathogen relative to the time period required by the host's endogenous immune response to clear the pathogen without administration of the disclosed agents. The treatment or amelioration of symptoms can be based on objective or subjective parameters, including the results of an examination by a physician. Accordingly, the term “treating”includes the administration of the agents or compositions disclosed in the present disclosure to alleviate, or to arrest or inhibit development of the symptoms or conditions associated with disease or condition (e.g., HBV infection). The term “therapeutic effect”refers to the amelioration, reduction, or elimination of the disease or condition, symptoms of the disease or condition, or side effects of the disease or condition in the subject. The term “therapeutically effective”refers to an amount of the composition that results in a therapeutic effect and can be readily determined.

In a specific embodiment, the composition is or comprises a nucleic acid molecule comprising a pathogen-associated molecular pattern (PAMP). The PAMP can comprise: a 5′ arm region comprising a terminal diphosphate; a poly uracil core comprising at least 8 contiguous uracil residues; and a 3′ arm region comprising at least 8 nucleic acid residues, wherein the 5′ most nucleic acid residue of the 3′ arm region is not a uracil and wherein the 3′ arm region is at least 30% uracil residues. Additional embodiments and features of the PAMP and/or nucleic acid comprising the PAMP that are encompassed in this aspect are described above in more detail and are not repeated here.

In another embodiment, the composition is or comprises a small molecule agent that induces RIG-I signaling. In some embodiments, the small molecule agent is or comprises a benzothiazol-derivative molecule, such as N-(6-benzamido-1,3-benzothiazol -2-yl)naphthalene-2-carboxamide. Additional embodiments and features of the small molecule agent that are encompassed in this aspect are described above in more detail and are not repeated here.

In some embodiments, the composition is formulated as an admixture of two or more therapeutic agents (e.g., comprising a first agent, a second agent, etc.). Alternatively, the method can comprise administering to the subject separate compositions (e.g., independently comprising a first agent, a second agent, etc.). The separate administrations can he simultaneous or coordinated such that the effects of the respective compositions are realized in overlapping, time-frames in the subject.

A representative example of a combination embodiment is a method comprising administering to the subject therapeutically effective amounts of a first agent and a second agent (in the same or different compositions), wherein:

the first agent is or comprises a nucleic acid molecule comprising: a 5′ arm region comprising a terminal triphosphate; a poly uracil core comprising at least 8 contiguous uracil residues; and a 3′ arm region comprising at least 8 nucleic acid residues, wherein the 5′ most nucleic acid residue of the 3′ arm region is not a uracil and wherein the 3′ arm region is at least 30% uracil residues; and

the second agent is or comprises a benzothiazol-derivative molecule, such as a small molecule comprising the chemical formula N-(6-benzamido-1,3-benzothiazol-2-yl)naphthalene-2-carboxamide.

The method can also include administering to the subject other anti-viral therapies, e.g., known therapies to treat HBV infection. In some embodiments, the treatment method further comprises administering to the subject a therapeutically effective amount of a with a reverse transcriptase inhibitor in addition to the at least one agent that induces IRF3 activation in the infected cell, as described above. For example, the method can further comprise administering a therapeutic amount of an NRTI, such as an NRTI selected from Lamivudine, Adefovir dipivoxil, Entecavir, Telbivudine, Tenofovir, Tenofovir alafenamide (TAF), Clevudine, Besivo, Zadaxin, Remdesivir, and the like.

In a specific embodiment, the method comprises administering to the subject therapeutically effective amounts of a first agent and a second agent (in a single composition or in separate compositions), wherein the first agent is or comprises a nucleic acid molecule comprising:

a 5′ arm region comprising a terminal triphosphate;

a poly uracil core comprising at least 8 contiguous uracil residues; and

a 3′ arm region comprising at least 8 nucleic acid residues, wherein the 5′ most nucleic acid residue of the 3′ arm region is not a uracil and wherein the 3° arm region is at least 30% uracil residues; and

wherein the second agent is or comprises an NRTI, such as Lamivudine, Adefovir dipiyoxil, Entecavir, Telbivudine, Tenofovir, Tenofovir alafenamide (TAF), Clevudine, Besivo, Zadaxin, Remdesivir, and the like. In further embodiments, the NRTI is Entecavir or Remdesivir.

In some embodiments, the composition or compositions are administered only once. Alternatively, the composition or compositions are administered multiple times according to a schedule established by a medical professional. Factors influencing the schedule include observed cccDNA levels, tolerance to the therapy, and the like.

In another aspect, the disclosure provides therapeutic compositions formulated for treating hepatitis B virus (HBV). This aspect also encompasses methods of administering the disclosed therapeutic compositions for treating and/or preventing HBV infection in a subject.

The therapeutic composition of this aspect comprises a RIG-I agonist, a vehicle for intracellular delivery, and a pharmaceutically acceptable carrier. In some embodiments, the RIG-I agonist is a nucleic acid molecule comprising a pathogen-associated molecular pattern (PAMP). Specific exemplary embodiments of the nucleic acid molecule and the PAMP are described in more detail above and are encompassed in this aspect. In other embodiments, the RIG-I agonist is or comprises a benzothiazol-derivative molecule. Exemplary embodiments are described in more detail above and are encompassed in this aspect. In one embodiment, the RIG-I agonist comprises the chemical formula N-(6-benzamide-1,3-benzothiazol-2-yl)naphthalene-2-carboxamide.

The active agent or agents can be incorporated into a vehicle to facilitate intracellular delivery. A variety of therapeutic delivery vehicles or systems are known and can be applied to the therapeutic composition. Delivery vehicles or systems can include particle formulations, such as emulsions, microparticles, immune-stimulating complexes (ISCOMs), nanoparticles, which can be, for example, particles and/or matrices, microspheres, liposomes, nanocapsules, and the like, which are advantageous for the delivery of antigens. The formulation and use of such delivery vehicles can be carried out using known and conventional techniques. In one embodiment, the disclosed PAMP-containing nucleic acid and any optional additional therapeutic agent are formulated into a liposomal delivery vehicle. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap solution including dissolved solutes within and/or between the lipid bilayers. Exemplary applications of liposomal formulations are described in Yallapu, U., et al., Liposomal Formulations in Clinical Use: An Updated Review, Pharmaceutics 9(2):1 (2017), incorporated herein by reference in its entirety.

In any of the above composition or treatment aspects and embodiments, the compositions or agents are appropriately formulated for the desired therapeutic administration according to known methods. For example, the compositions can be appropriately formulated for preferred routes of administration according to known methods. The pharmaceutical composition can be formulated for delivery by any route of systemic administration (e.g., intramuscular, intradermal, subcutaneous, subdermal, transdermal, intravenous, intraperitoneal intracranial, intranasal, mucosal, anal, vaginal, oral, or buccal route, or they can be inhaled). Certain routes of administration are particularly appropriate for pharmaceutical compositions intended to induce, at least, elements of an innate immune response. In particular, transdermal administration, intramuscular, subcutaneous, and intravenous administrations are particularly appropriate.

The formulations suitable for introduction of the therapeutic compositions vary according to route of administration. Formulations suitable for parenteral administration, such as, for example, by intraarticular (in the joints), intravenous, intramuscular, intradermal, intraperitoneal, intranasal, and subcutaneous routes, include aqueous and non-aqueous, isotonic sterile, injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures, thereof and in oils. Under ordinary conditions of storage and use, such preparations can contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the -form should be sterile and should be fluid to the extent that easy syringability exists. It should be stable under the conditions of manufacture and storage and should be preserved against the contaminating action of microorganisms, such as bacteria and fungi.

As used herein, “carrier”includes any and all solvents, dispersion media, diluents, antibacterial and antifungal agents, isotonic and absorption delaying agents, buffers, carrier solutions, suspensions, colloids, and the like. The carrier can be a solvent or dispersion medium containing, for example, water, ethanol, polyol (e.g., glycerol, propylene glycol, mannitot, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The phrase “pharmaceutically-acceptable”refers to molecular entities and compositions that do not produce an allergic or similar untoward reaction when administered to a subject (e.g., human).

### General Definitions

Unless specifically defined herein, all tennis used herein have the same meaning as they would to one skilled in the art of the present disclosure. Practitioners are particularly directed to Ausubel, F. M., et al. (eds.), Current Protocols in Molecular Biology, John Wiley & Sons, New York (2010), Coligan, J. E, et al. (eds.), Current Protocols in Immunology, John Wiley & Sons, New York (2010), Mirzaei, H. and Carrasco, M. (eds.), Modern Proteomics—Sample Preparation, Analysis and Practical Applications in Advances in Experimental Medicine and Biology, Springer international Publishing, 2016, and Comai, L. et al., (eds.), Proteomic: Methods and Protocols in Methods in Molecular Biology, Springer International Publishing, 2017, for definitions and terms of art.

For convenience, certain terms employed herein, in the specification, examples and appended claims are provided here. The definitions are provided to aid in describing particular embodiments and are not intended to limit the claimed invention, because the scope of the invention is limited only by the claims.

The use of the term “or”in the claims is used to mean “and/or”unless explicitly indicated to refer to alternatives only or the alternatives are mutually exclusive, although the disclosure supports a definition that refers to only alternatives and “and/or.”

The words “a”and “an,”when used in conjunction with the word “comprising” in the claims or specification, denotes one or more, unless specifically noted.

Unless the context clearly requires otherwise, throughout the description and the words “comprise,” “comprising,”and the like, are to be construed in an inclusive sense as opposed to an exclusive or exhaustive sense, which is to indicate, in the sense of “including, but not limited to.”Words using the singular or plural number also include the plural and singular number, respectively. Additionally, the words “herein,”“above,”and “below,”and words of similar import, when used in this application, shall refer to this application as a whole and not to any particular portions of the application. The word “about”indicates a number within range of minor variation above or below the stated reference number. For example, in some embodiments, the term “about”refers to a number within a range of 10%, 9%, 8%, 7%, 6%, 5%, 4%, 3%, 2%, or 1% above and/or below the indicated reference number.

As used herein, the term “polypeptide”or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed, Additionally, it is understood that the embodiments described herein can be implemented using any suitable material such as those described elsewhere herein or as known in the art.

Publications cited herein and the subject matter for which they are cited are hereby specifically incorporated by reference in their entireties.

The following examples are provided to illustrate certain features and/or embodiments of the disclosure. This example should not be construed to limit the invention to the particular features or embodiments described.

### EXAMPLES

### Example 1

This example describes the demonstration that induction of RIG-I signaling pathway destabilizes cccDNA and prevents formation of new cccDNA in hepatocytes; providing a strategy to eradicate the cccDNA and corresponding hepatitis B viral infection.

### Introduction

Acute virus infection typically triggers intracellular innate immune activation leading to induction of intracellular antiviral defenses. This process serves to control viral replication and spread from the site of infection, and to modulate the adaptive immune response for systemic virus control. Innate immune activation occurs via host cell sensing of viral pathogen associated molecular patterns (PAMP) embedded in viral replication products, including viral nucleic acid. PAMPs are sensed by cellular pattern recognition receptors (PRRs). PRRs that sense virus infection include Toll-like receptors (TLR), NOD-like receptors (NLR), intracellular DNA sensors cGAS, STING, IFI16, DAI and others, as well as the RIG-I-like receptors (RLRs) including retinoic-acid inducible gene-I (RIG-I) and melanoma differentiation antigen 5 (MDA5). Each PRR detects specific PAMPs derived from the incoming virus or viral replication products, while certain host cell nucleic acids can also trigger PRR signaling when produced during virus infection. Induction of TLR, RLR, or STING signaling drives the downstream activation of latent transcription factors including interferon regulatory factor (IRF)3 and NF-κB to promote the expression of antiviral effector genes and immune regulatory genes including chemokines, IFNs, and other immune regulatory cytokines. Remarkably, acute HBV infection of primary human hepatocytes (PHHs) neither activates nor inhibit PRR signaling of innate immunity, thus reinforcing the notion that HBV is a “stealth”virus as previously shown in vivo in a nonhuman primate infection model.

Previous studies show that RIG-I signaling in response to PAMP RNA can direct iterate immune activation and antiviral defenses that suppress replication of hepatitis C virus (HCV) that also causes chronic hepatitis. The HCV PAMP is a 100 in poly uridine/cytosine (poly-U/UC) motif with the HCV genome 3′ nontranslated region. When introduced into cells as a 5′ppp synthetic RNA, the poly-U/UC PAMP specifically activates RIG-I to drive IRF3 activation and antiviral innate immunity that suppresses HCV infection in vitro and activates hepatic innate immunity in vivo. Moreover, small molecule benzothiazols, or 5′ppp RNA ligands that bind and activate RIG-I or induce IRF3 activation and innate immunity, have been identified to therapeutically suppress RNA virus infection and enhance the immune response. Together, these studies show that the RIG-I pathway is functional in hepatocytes in which targeted activation of RIG-I confers potent antiviral actions that are fully operational in the liver. However, it is unknown how the direct targeting and activation of RIG-I and IRF3 impacts HBV infection.

Here, targeted RIG-I activation was evaluated in the treatment of HBV infection in vitro RIG-I signaling triggered by poly-U/UC PAMP RNA or small molecule activator of RIG-I directed robust IRF3 activation and RIG-I-dependent antiviral actions to suppress cccDNA levels. The results demonstrate that the RIG-I response through IRF3 serves to reduce the half-life (t_{1/2}) of cccDNA to impart cccDNA decay kinetics, and blockage of RC DNA formation and concomitant suppression of HBsAg secretion in HepG2 cells that ectopically expressing human sodium/taurocholate cotransporting polypeptide (hNTCP) (HepG2-hNTCP), in differentiated HepaRG (dHepaRG) cells, and in primary human hepatocytes (PHHs). Targeting RIG-I does not promote cell toxicity. Remarkably, when combined with a therapeutic nucleoside reverse transcriptase inhibitor (NRTI), entecavir, poly-U/UC PAMP, treatment rapidly depleted established cccDNA pools. Thus, targeted RIG-I activation and processes that activate IRF3-directed innate immune activation offer novel and effective therapeutic approaches toward HBV cure.

### Results

### RIG-I and IRF3 Agonists Trigger Innate Immune Activation in Hepatocytes

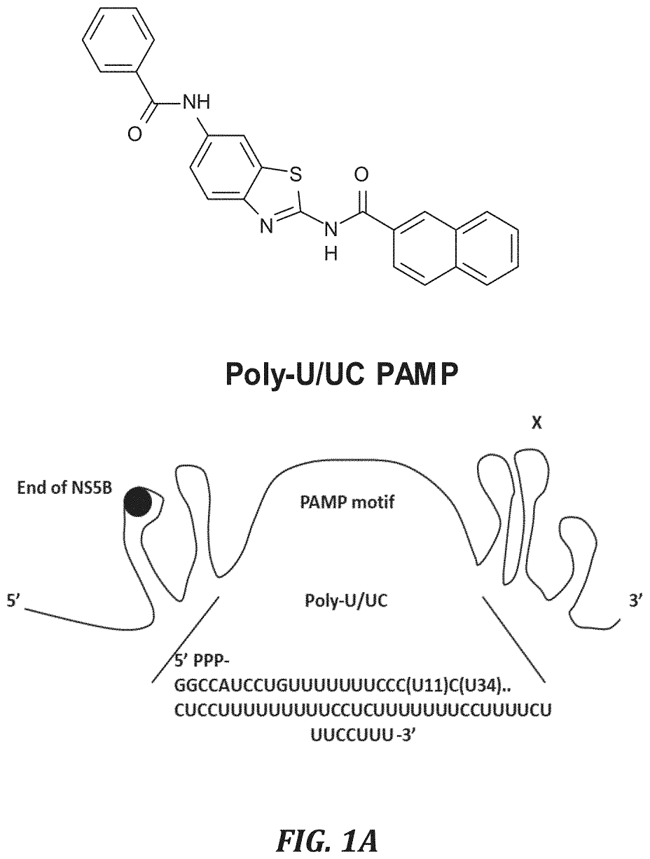

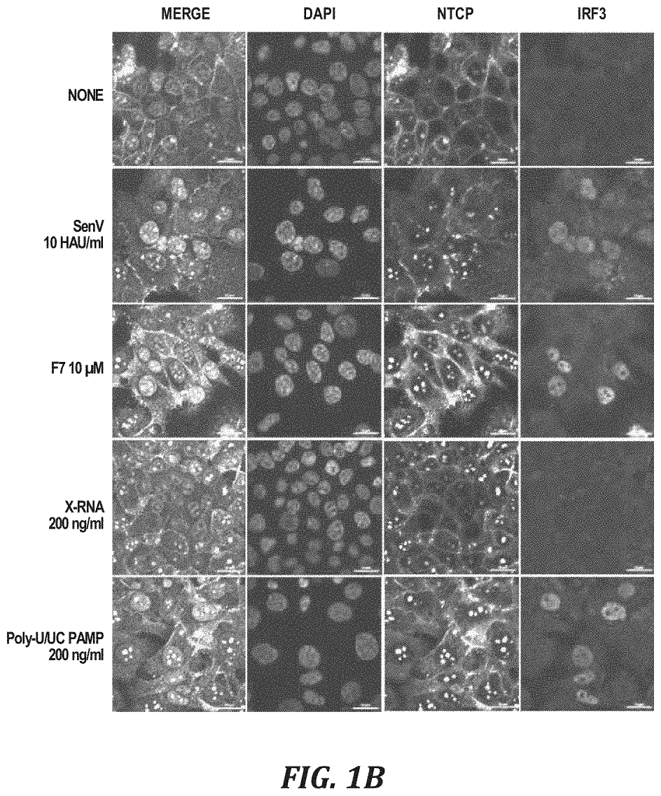

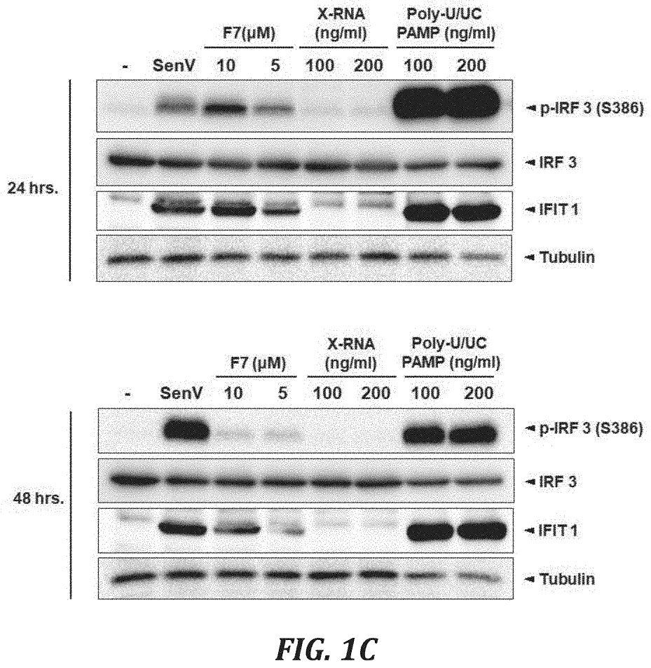

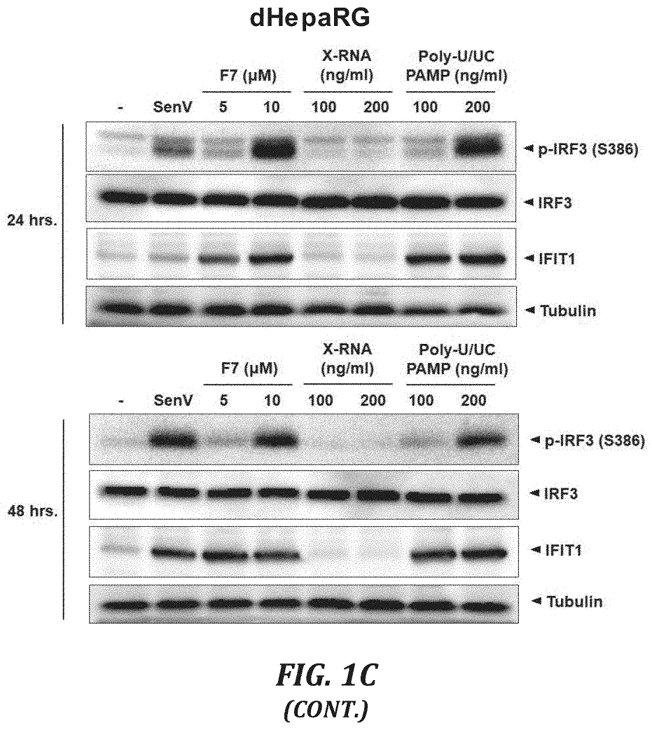

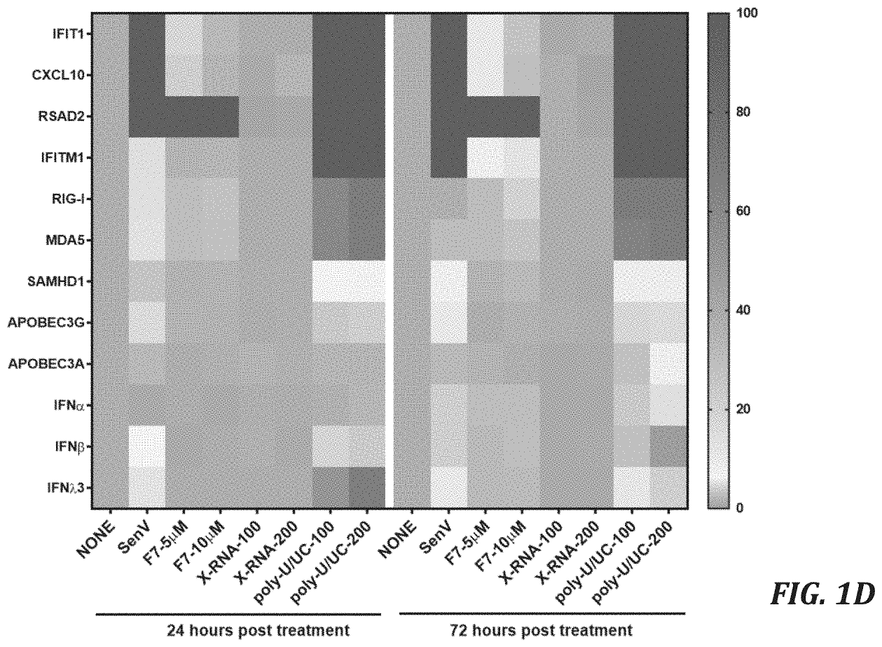

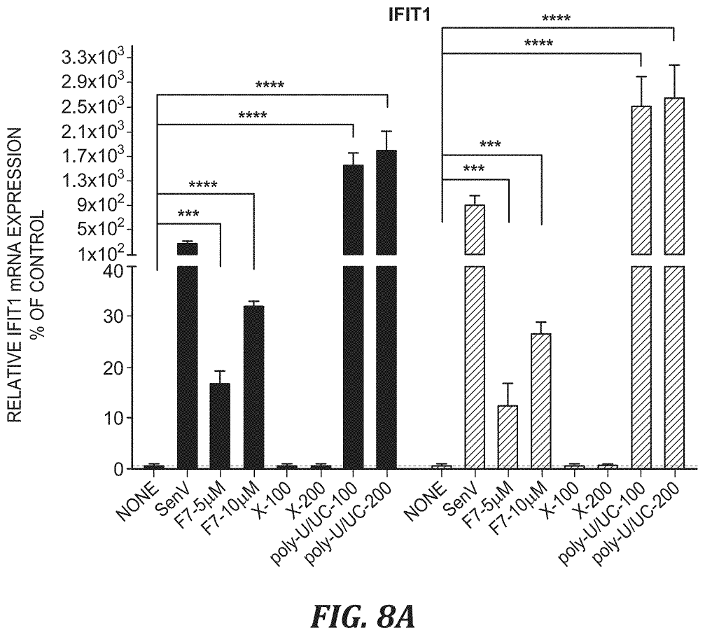

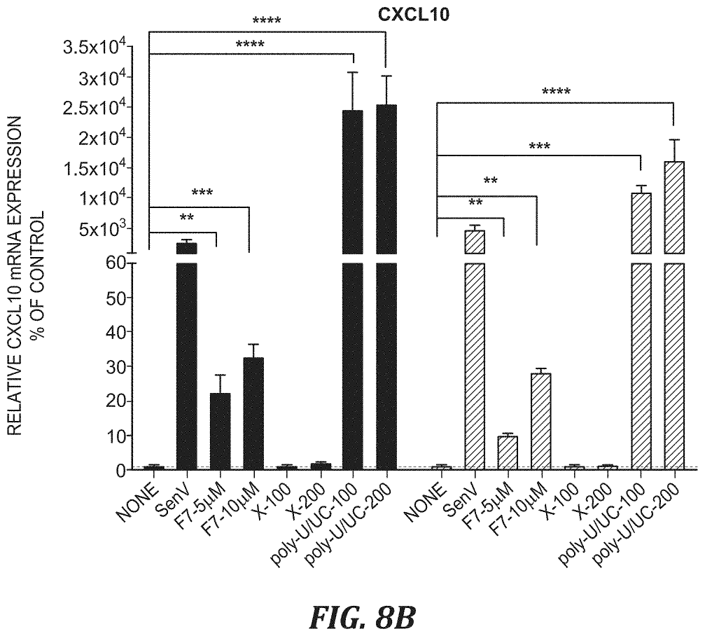

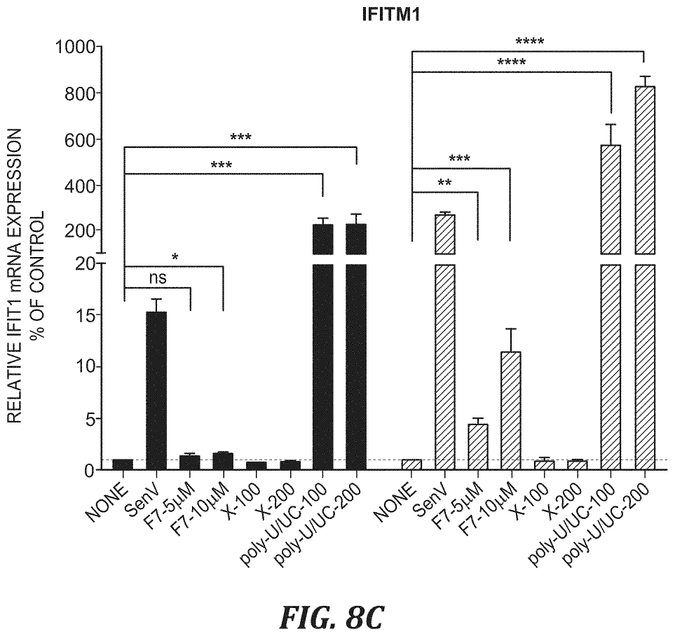

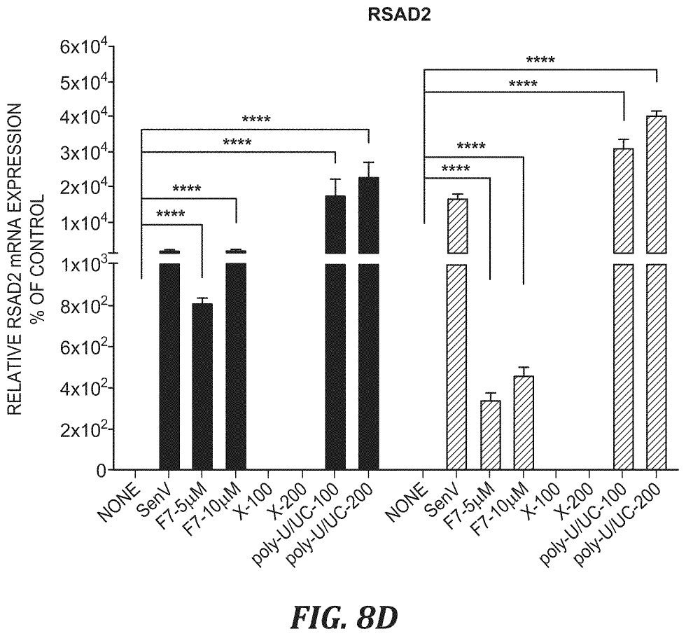

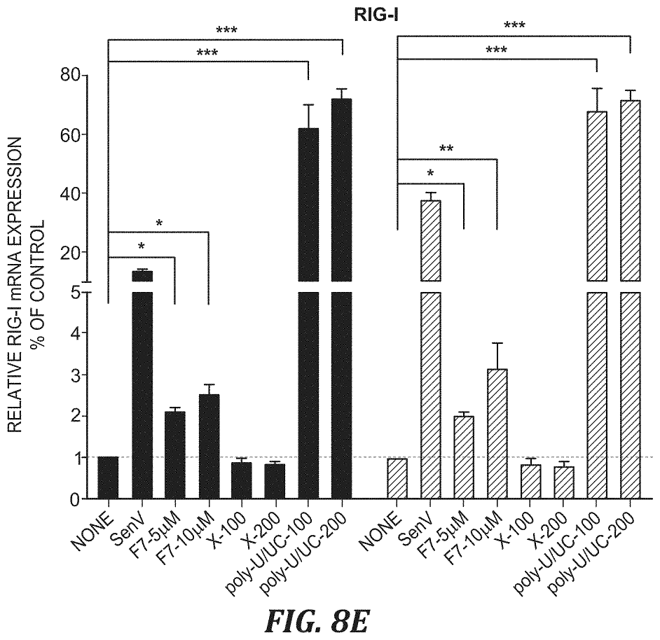

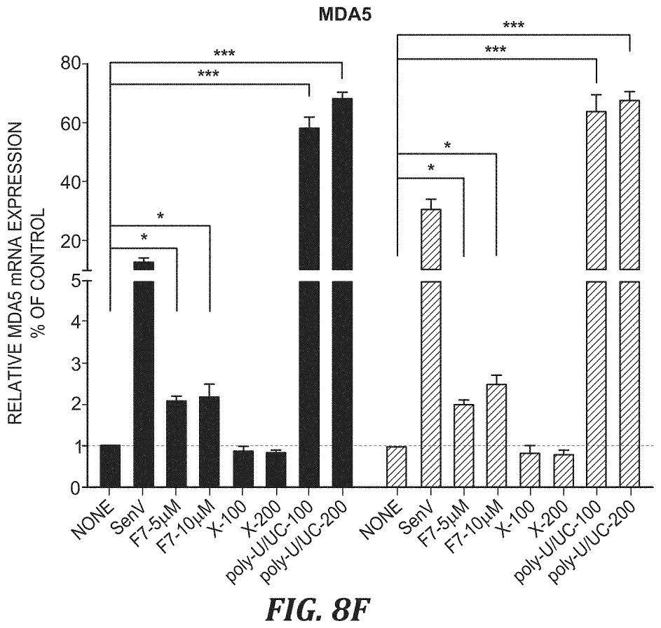

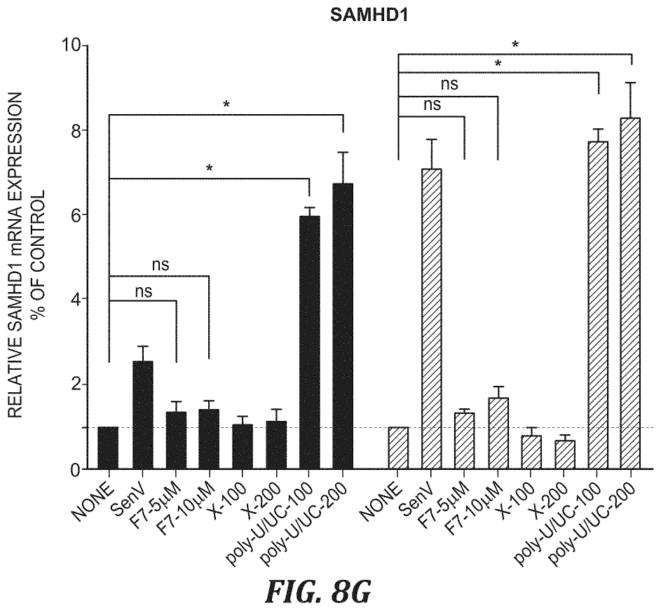

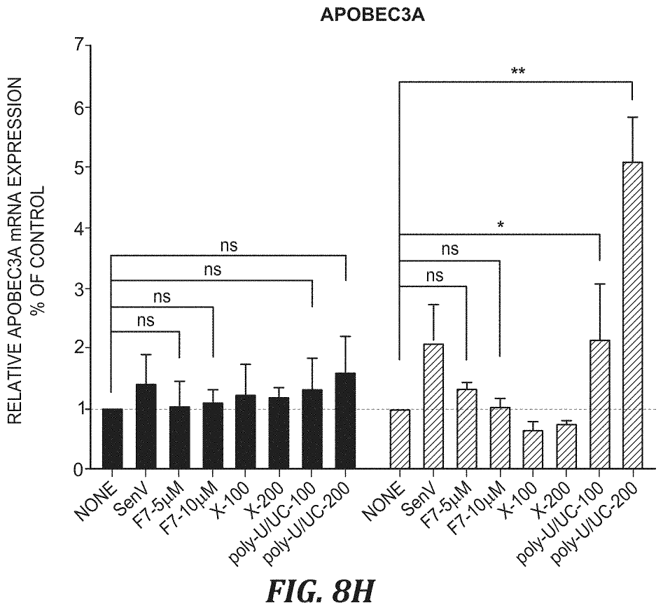

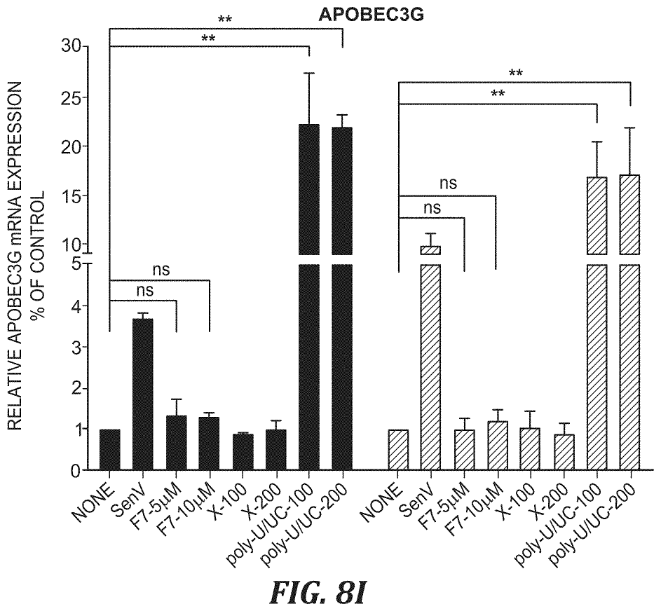

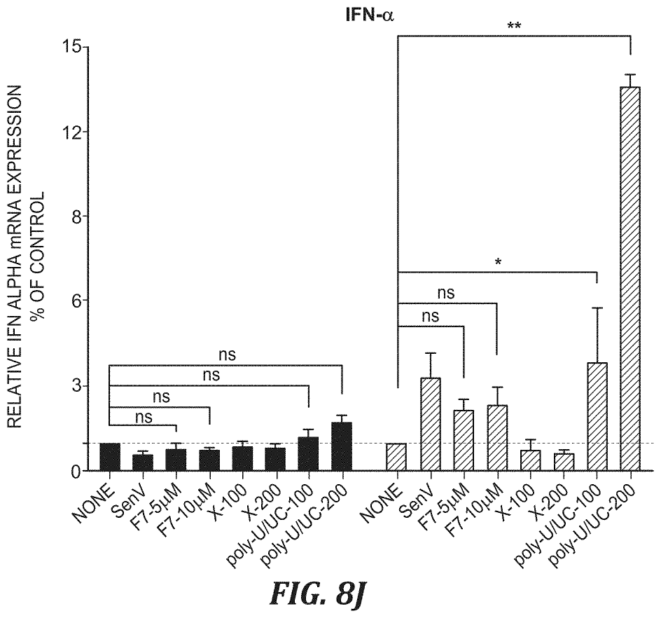

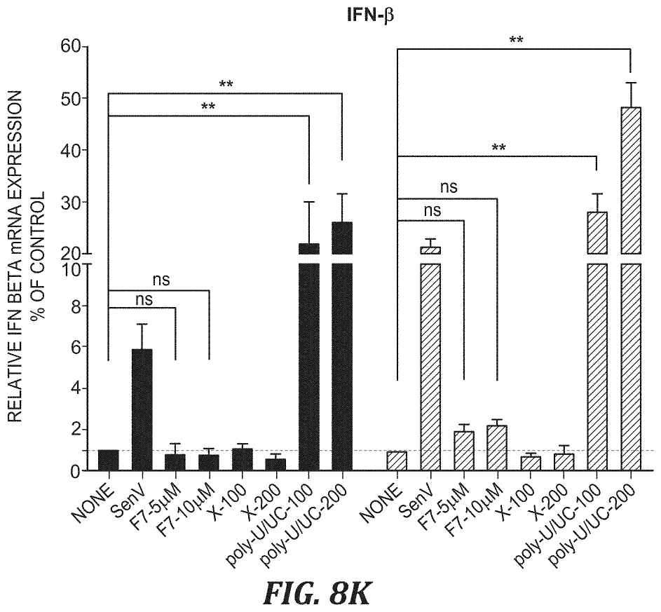

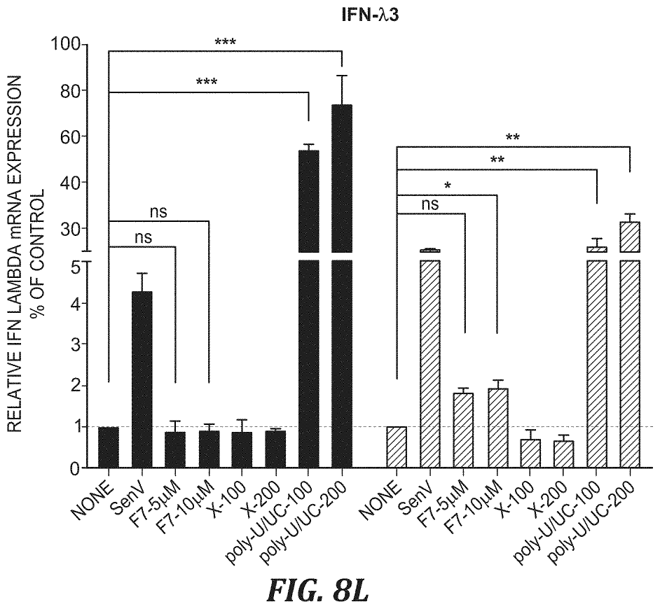

Small-molecule agonists of IRF3 were previously identified that confer innate immune activation leading to induction of IRF3-target genes and antiviral action against a range of RNA viruses (Bedard, K. M., et al. (2012). Isoflavone agonists of IRF-3 dependent signaling have antiviral activity against RNA viruses. Journal of virology 86, 7334-7344; Pattabhi, S., et al. (2016). Targeting innate Immunity for Antiviral Therapy through Small Molecule Agonists of the RLR Pathway. Journal of virology 90, 2372-2387; Probst, P., et al. (2017). A small-molecule IRF3 agonist functions as an influenza vaccine adjuvant by modulating the antiviral immune response. Vaccine 35, 1964-1971). Based on the published structure U.S. Pat. No. 9,884,876, (N-(6-benzamido-1,3-benzothiazol-2-yl)naphthalene-2-carboxamide), referred here as F7, was produced for analyses of anti-HBV activity (FIG. 1A). Similarly, a ˜100 nt PAMP motif was identified within the HCV genome comprising 5′ppp and the poly U/UC region of viral RNA that is specifically recognized by RIG-I and confers RIG-I signaling of IRF3 activation leading to antiviral gene expression (Saito, T., et al. (2008). Innate immunity induced by composition-dependent RIG-I recognition of hepatitis C virus RNA. Nature 454, 523-527; and Schnell, G., et al. (2012). Uridine composition of the poly-U/UC tract of HCV RNA defines non-self recognition by RIG-I. PLoS pathogens 8, e1002839 (FIG. 1A, lower panel). The therapeutic actions of F7 and the poly-U/UC PAMP against HBV infection were evaluated. First, HepG2-hNTCP cells were treated with 10 μM of F7 or 200 ng/ml of poly-U/UC PAMP formulated in liposome for 24 hours. Control cells were treated with DMSO or infected with Sendai virus (SenV; a potent activator of RIG-I-dependent signaling), or transfected with 200 ng/ml of X-RNA in liposome, a non-PAMP/non-signaling 5′ppp-containing 100 nt RNA motif from the HCV genome with similar mass to the poly-U/UC PAMP (Saito et at. (2008), supra). Similar to SenV control, both F7 and poly-U/UC PAMP but neither DMSO nor X RNA treatment specifically induced innate immune activation as marked by IRF3 translocation into the nucleus (FIG. 1B). Immunoblot analysis demonstrated that F7 and poly-U/UC PAMP but not XRNA treatment specifically induced IRF3 phosphorylation and the expression of IFIT1, an IRF3-target gene (Fenster, V., and Sen, G. C. (2011). The ISG56/IFIT1 gene family. Journal of interferon & cytokine research: the official journal of the International Society for Interferon and Cytokine Research 31, 71-78; and Fensterl, V., and Sen. G. C. (2015). Interferon-induced Ifit proteins: their role in viral pathogenesis. Journal of virology 89, 2462-2468) in a dose-dependent manner in both HepG2-hNTCP and dHepaRG cells (FIG. 1C). mRNA expression was also assessed across a panel of innate immune response genes, including IFNs, ISGs, and direct IRF3-target genes, for response to F7 or poly-U/UC PAMP (FIG. 1D; FIGS. 8A-8L). While poly-U/UC PAMP treatment also induced type I and type III IFN and ISG expression. F7 treatment induced only IRF3-target gene expression. This difference is consistent with the signaling properties of each molecule and the nature of IFN expression, as IFN expression relies on activation of both IRF3 and NF-kB, whereas F7 specifically activates IRF3 but not NE-kB (Bedard et al. (2012), supra). In contrast, the poly-U/UC PAMP triggers RIG-I signaling of both transcription factors to induce IRF3-target genes, IFNs and hence ISGs (Saito, T., et al. (2008), supra; Schnell, G., et al. (2012), supra). Notably, type I IFN, SAMHD1, APOBEC3A, and APOBEC3G, which were induced by poly-U/UC PAMP treatment, have demonstrated antiviral activity against HBV infection (Bonvin, M., et al. (2006). Interferon-inducible expression of APOBEC3 editing enzymes in human hepatocytes and inhibition of hepatitis B virus replication. Hepatology (Baltimore, Md.) 43, 1364-1374: Chen, Z., et al. (2014). Inhibition of Hepatitis B virus replication by SAMHD1. Biochemical and biophysical research communications 450, 1462-1468; Lucifora, J., et al. (2014). Specific and nonhepatotoxic degradation of nuclear hepatitis B virus cccDNA. Science (New York, N.Y.) 343, 1221-1228). Taken together, these results show that F7 and poly-U/UC PAMP induce innate immune activation in treated cells that initiates with IRF3 activation and the induction of IRF3-target genes.

### IRF3 Activation Suppresses HBV cccDNA Formation

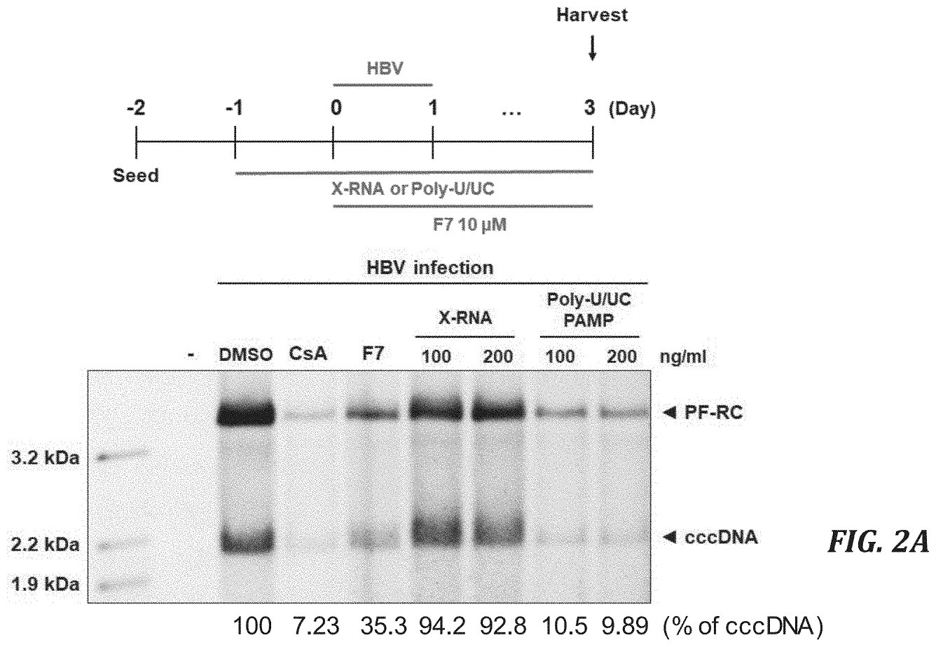

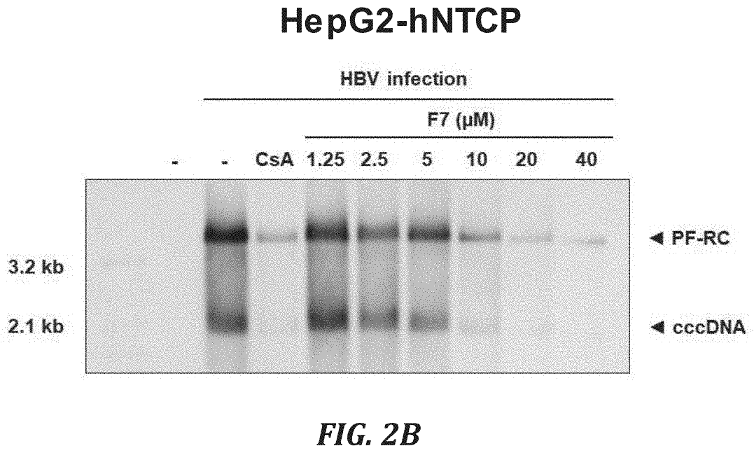

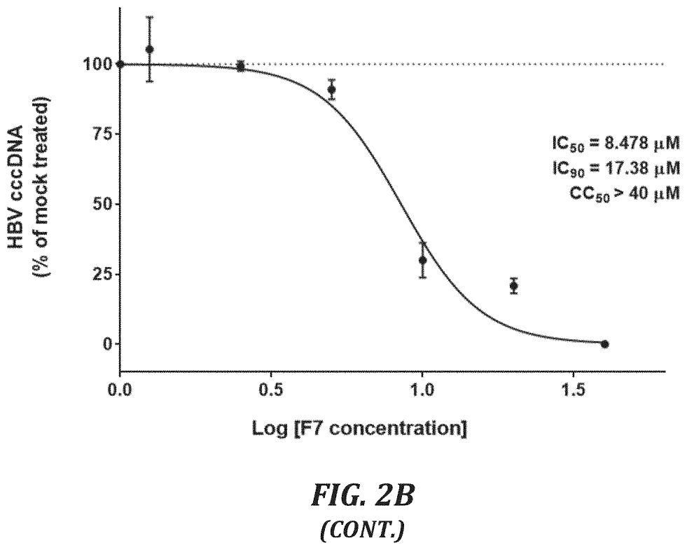

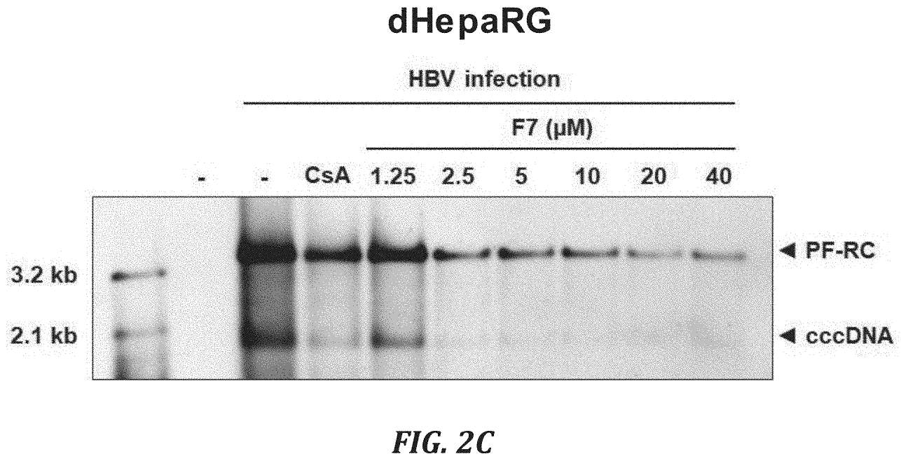

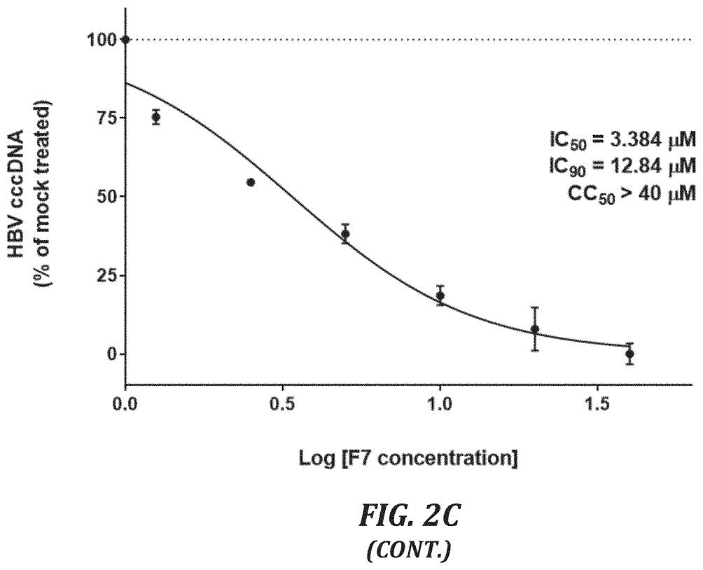

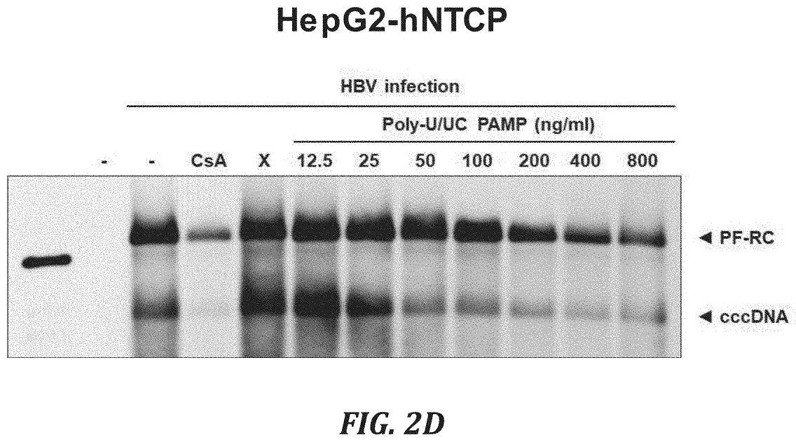

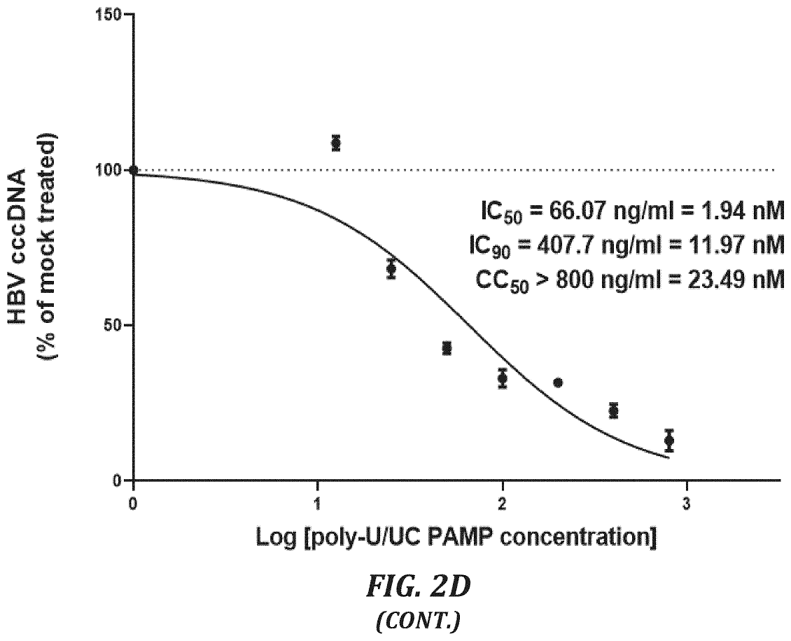

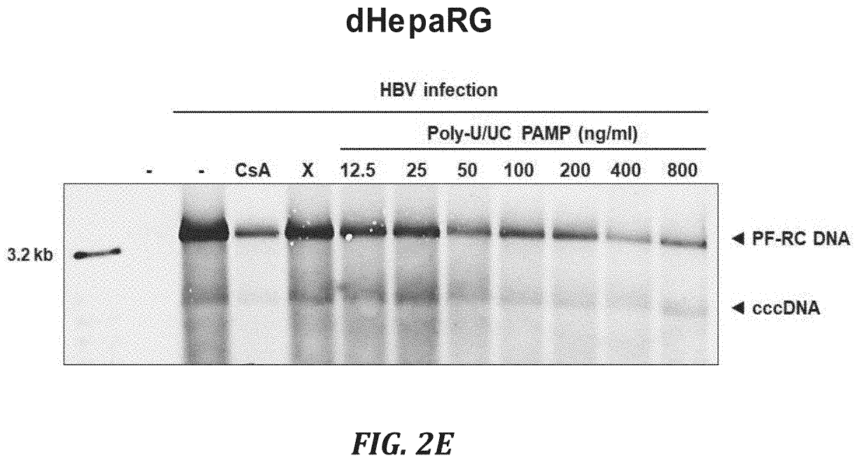

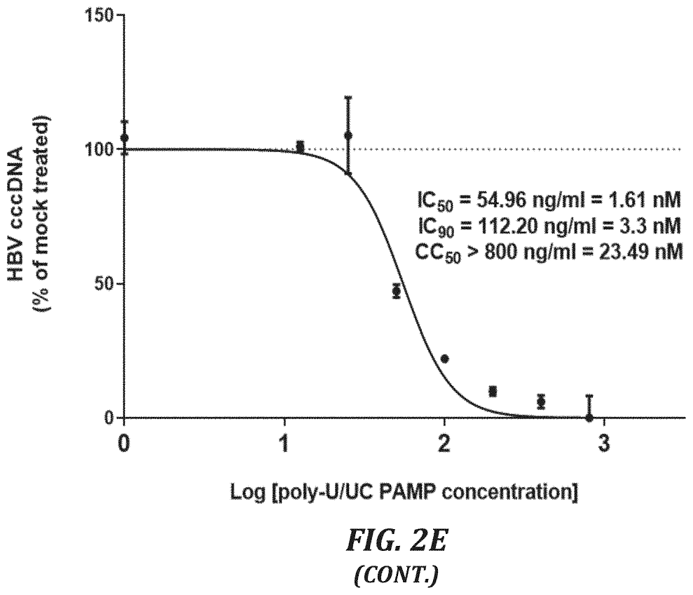

To determine how IRF3 activation impacts HBV infection and production of cccDNA in infected cells, HepG2-hNTCP cells were treated with F7 or poly-U/UC PAMP (100 ng/ml [=2.94 nM] or 200 ng/ml [=5.87 nM]) following HBV infection. Cyclosporin A (CsA) treatment was employed as an HBV entry inhibitor antiviral control (Watashi, K., et al. (2014). Cyclosporin A and its analogs inhibit hepatitis B virus entry into cultured hepatocytes through targeting a membrane transporter, sodium taurocholate cotransporting polypeptide (NTCP). Hepatology (Baltimore, Md.) 59, 1726-1737). Cells were harvested 3 days post-infection (dpi) and extracts were prepared using Hirt extraction methods for isolating protein-free DNA, as shown in FIG. 2A (Guo, H., et al. (2007). Characterization of the Intracellular Deproteinized Relaxed Circular DNA of Hepatitis B Virus: an Intermediate of Covalendy Closed Circular DNA Formation. Journal of Virology 81, 12472-12484 Hirt, B. (1967). Selective extraction of polyoma DNA from infected mouse cell cultures. Journal of molecular biology 26, 365-369). Southern blot analysis revealed that F7 and poly-U/UC PAMP treatment suppressed HBV cccDNA formation compared to the level of expression for the DMSO, or X-RNA treated controls. Additionally, the level of protein free-relaxed circular (PF-RC) DNA, the precursor of cccDNA, was also markedly decreased by treatment with F7 or poly-U/UC PAMP. As both F7 and poly-U/UC PAMP activate IRF3, these results link the IRF3 response with suppression of cccDNA formation in HBV infected hepatocytes.

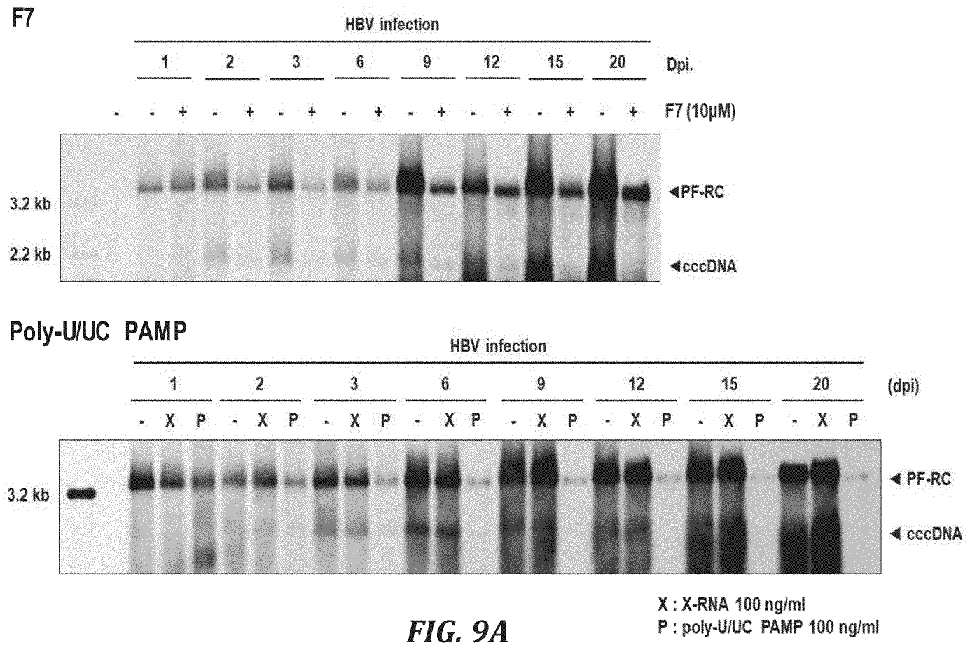

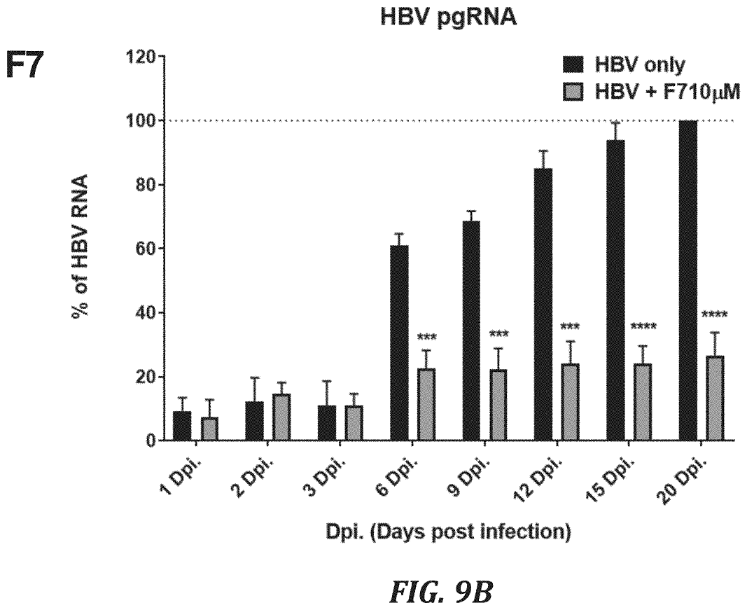

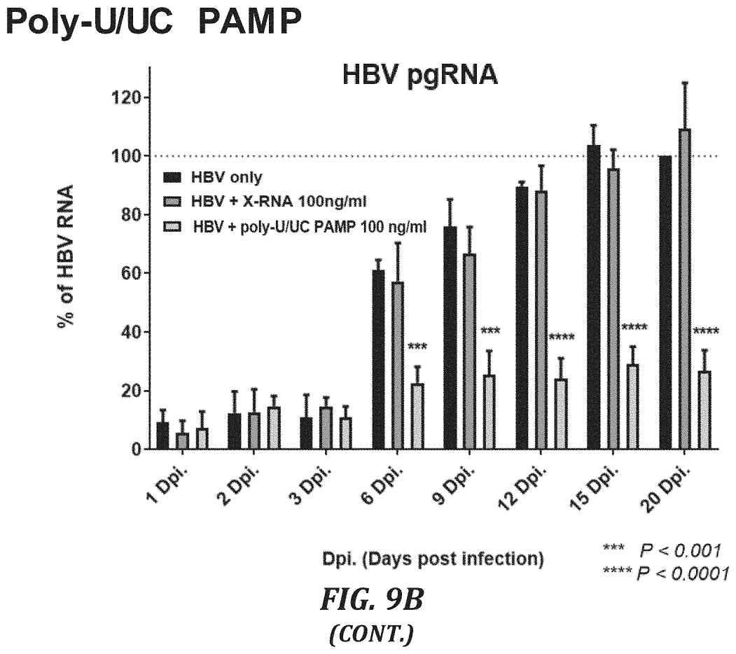

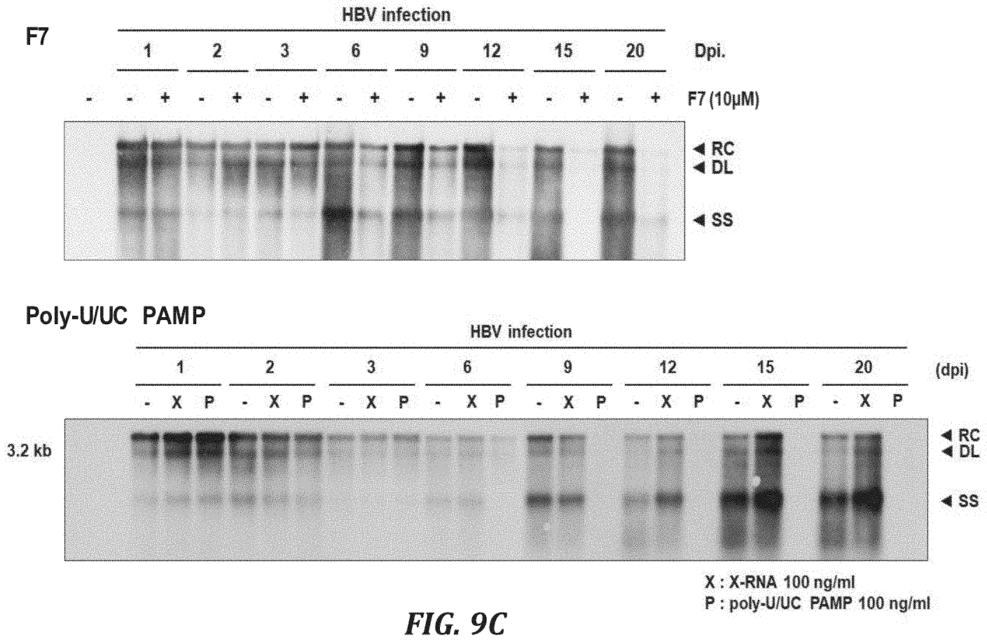

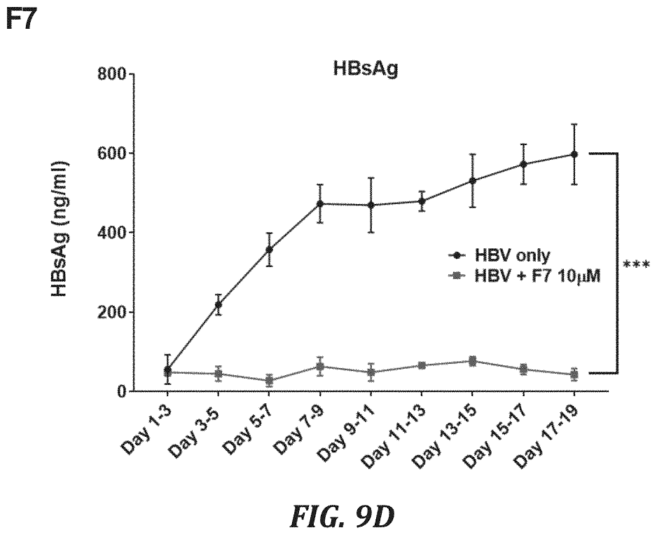

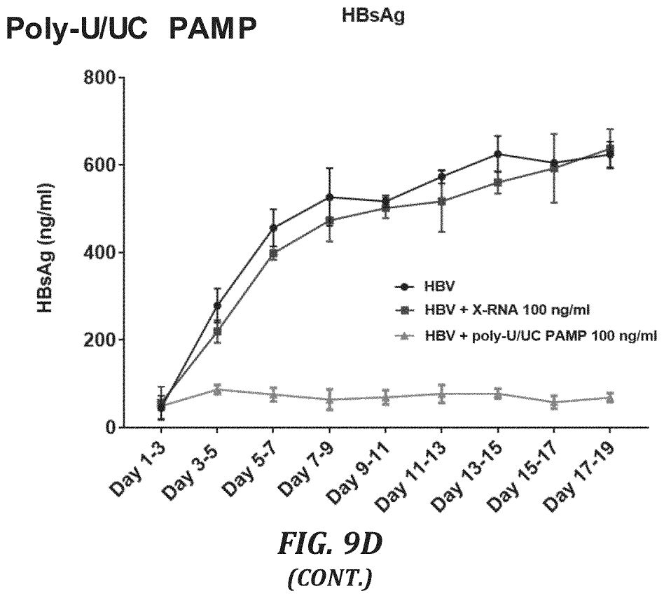

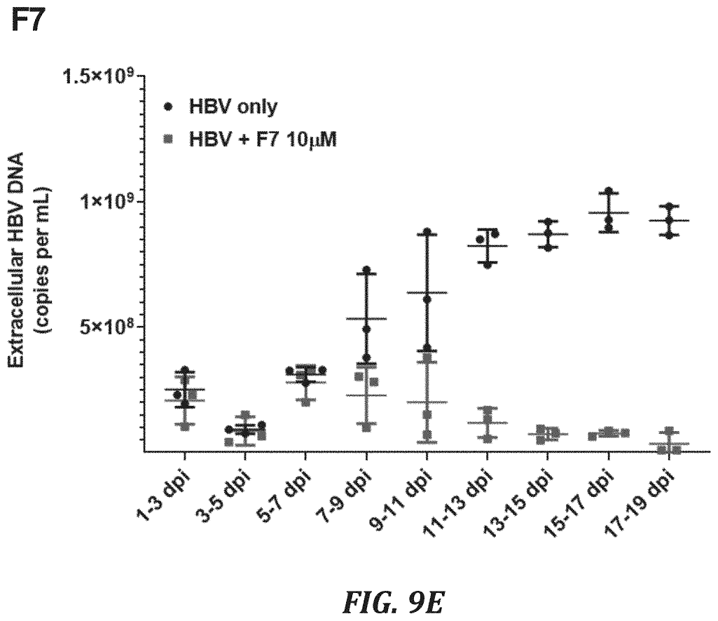

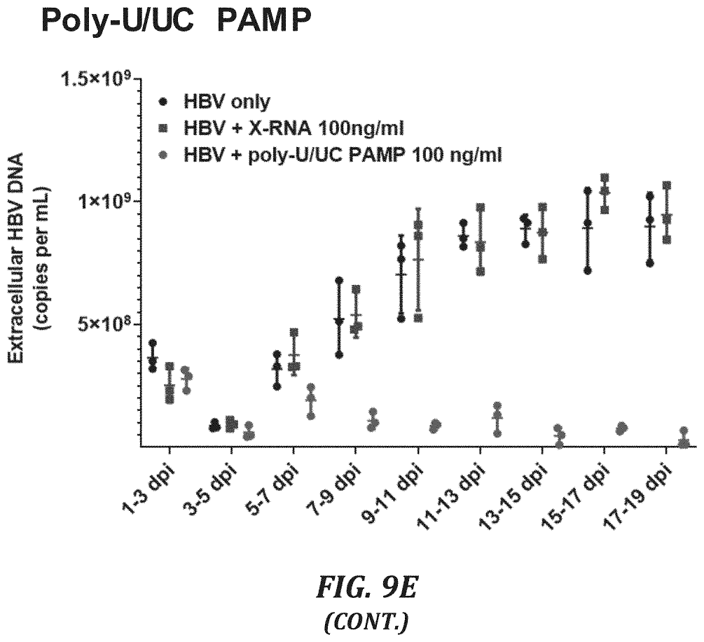

To further determine how the activation of IRF3 impacts the HBV replication cycle, the expression of viral DNA, viral RNA, extracellular HBV DNAs and secreted HBsAg were analyzed over an infection/treatment time course. As shown in FIG. 9A, PF-RC DNA was first detected by 1 dpi. cccDNA synthesis occurred by 2 dpi in control-treated cells, and PF-RC and cccDNA both accumulated over the 20 dpi time course. However, the production of cccDNA was markedly suppressed in cells treated with F7 or poly-U/UC PAMP within 2 dpi. Production of pgRNA across the infection/treatment time course showed that it accumulated from 6 dpi to 20 dpi in the nontreatment control cells but was significantly suppressed in cells treated with F7 or poly-U/UC PAMP (FIG. 9B). The level of capsid-associated intracellular HBV DNA intermediates of reverse transcription produced over the HBV infection/treatment time course were also measured. It was found that relaxed circular (RC) DNA, double stranded linear (DL) DNA, and single stranded (SS) DNA products accumulated from 6 to 20 dpi in control/nontreated cells. However, F7 or poly-U/UC PAMP treatment markedly decreased the levels of these DNA species. Interestingly, the level of incoming viral capsid-associated HBV DNAs (observed from 1-2 dpi) were not affected by treatment but instead their production was suppressed within 6 dpi (FIG. 9C). Suppression of HBV DNA and RNA overall was associated with significant reduction in the level of secreted HBsAg in culture supernatant from cells treated with F7 or poly-U/UC PAMP (FIG. 9D). Importantly, both F7 and poly-U/UC PAMP treatment resulted in a block in de novo HBV production revealed by reduction of extracellular HBV DNA (FIG. 9E). These results show that F7 and poly -U/UC PAMP treatment impact HBV replication at steps following cccDNA synthesis to impact transcription of viral RNA, reverse transcribed HBV DNAs, and production of progeny virions.

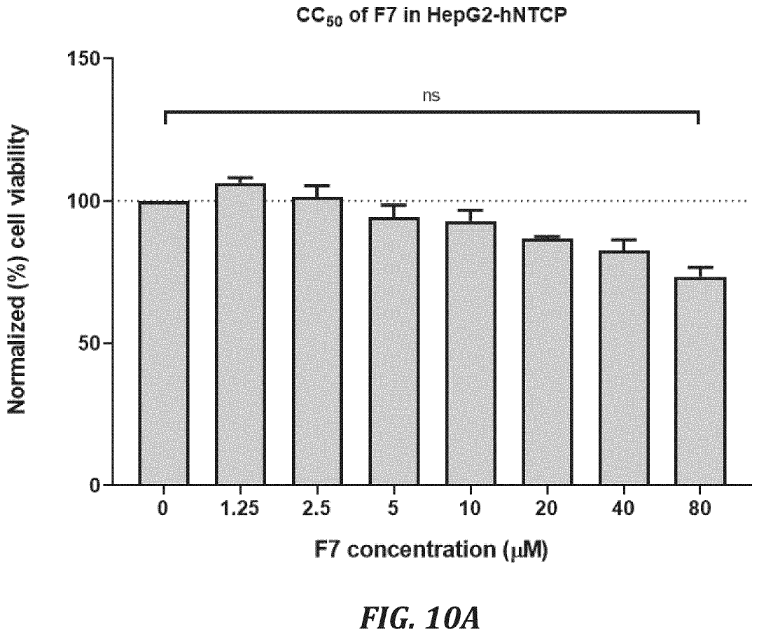

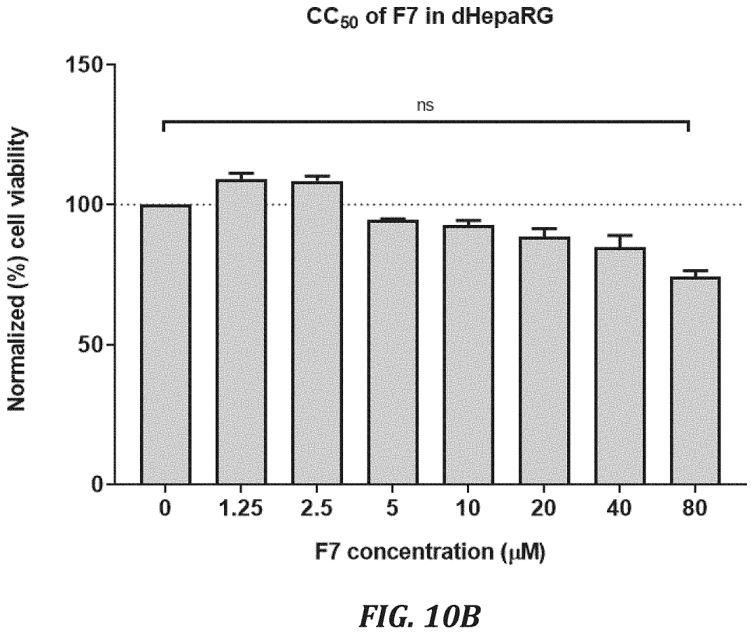

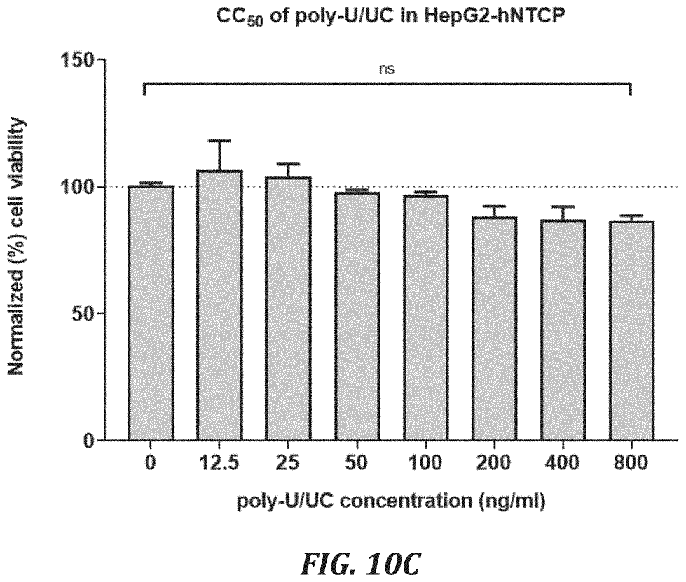

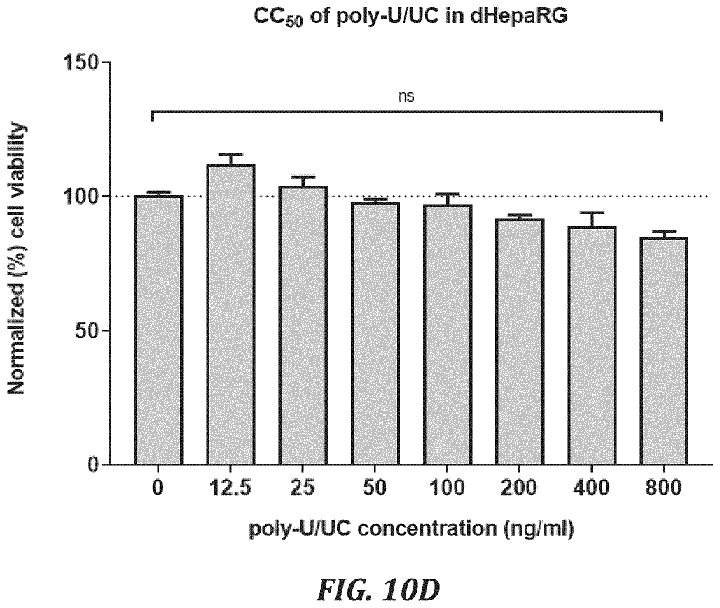

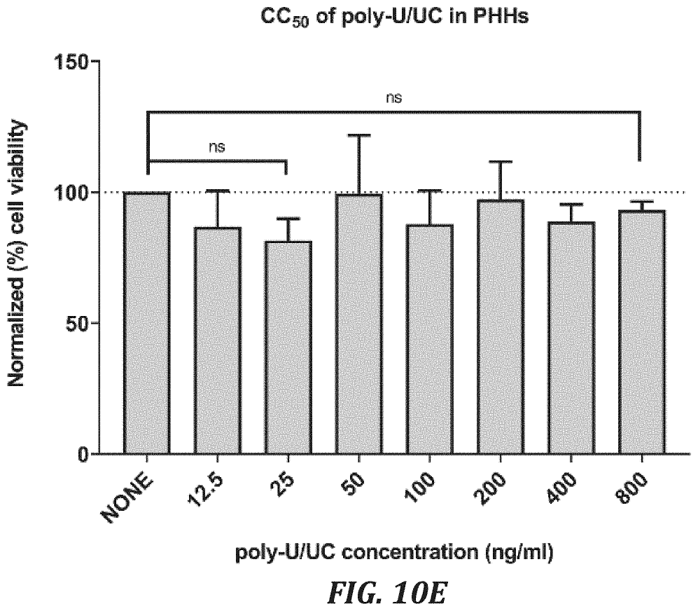

To assess the antiviral activity of F7 and poly-U/UC PAMP against HBV, dose-response analyses was conducted. To determine the values of 90% of maximal Inhibitory concentration (IC₉₀) and half-maximal Inhibitory concentration (IC₅₀), as well as the cytotoxic concentration, CC₅₀, of F7 and poly-U/UC, PAMP, two different cell lines were used, HepG2-hNTCP and dHepaRG cells. Cultures were treated with increasing concentrations of F7 or poly-U/UC followed by HBV infection, then harvested at 3 dpi. Southern blot and qPCR analysis showed that cccDNA level was linearly reduced by increasing concentrations of F7 (FIGS. 2B and 2C) and poly-U/UC PAMP (FIGS. 2D and 2E). IC₉₀and IC₅₀ values were defined for F7 of approximately 17.38 μM and 8.48 μM in HepG2-hNTCP cells, and 12.84 μM and 3.38 μM in differentiated HepaRG cells. The 50% cytotoxic concentration (CC₅₀), as measured by ATP release from treated cells, was over 40 μM (FIGS. 10A and 10B). The IC₉₀ and IC₅₀ values of poly-U/UC PAMP were determined that to be approximately 11.97 nM and 1.94 nM, respectively, in HepG2-NTCP cells, 3.3 nM and 1.61 nM in dHepaRG respectively, with CC₅₀ over 23.49 nM (=800 ng/ml) (FIGS. 10C and 10D).

### Suppression of de novo HBV cccDNA Synthesis

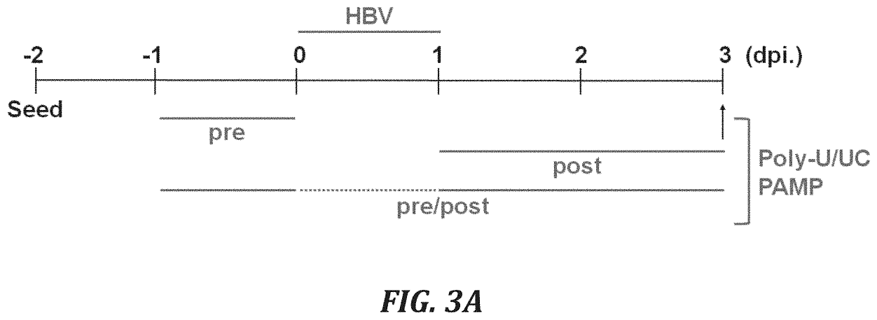

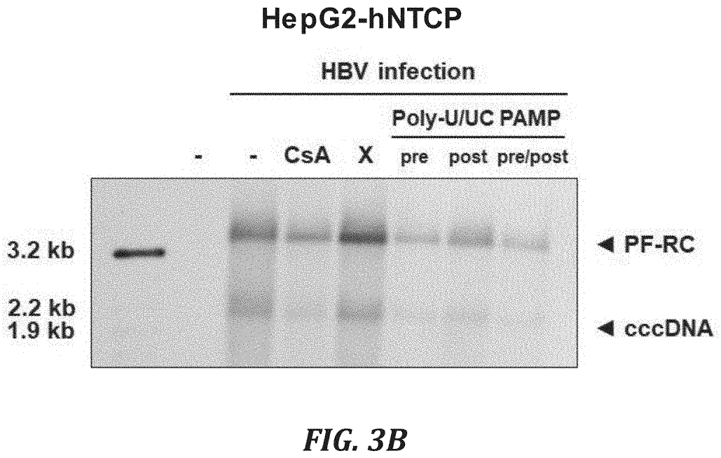

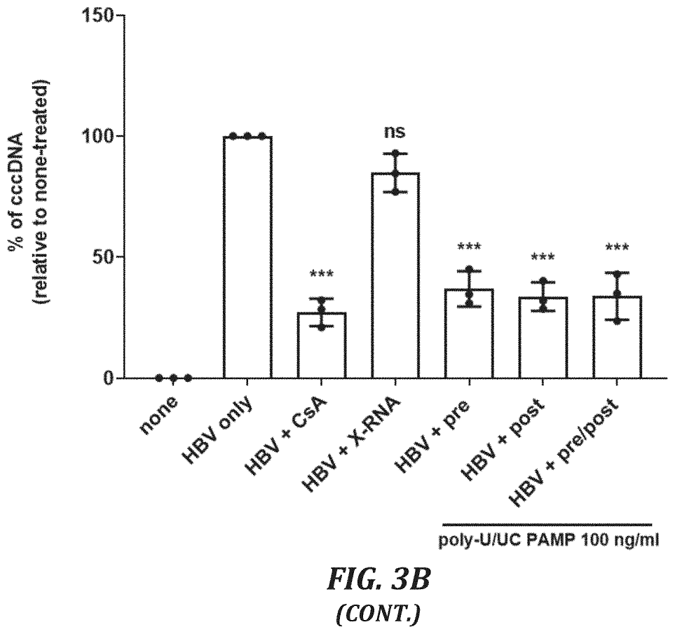

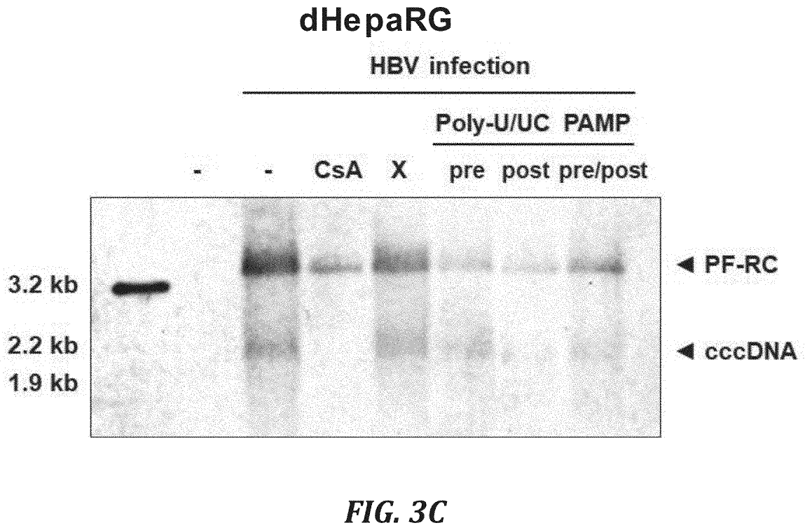

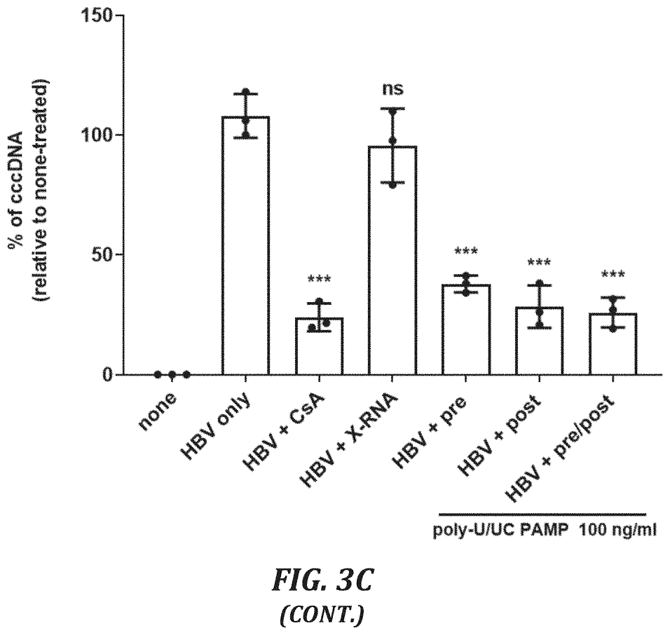

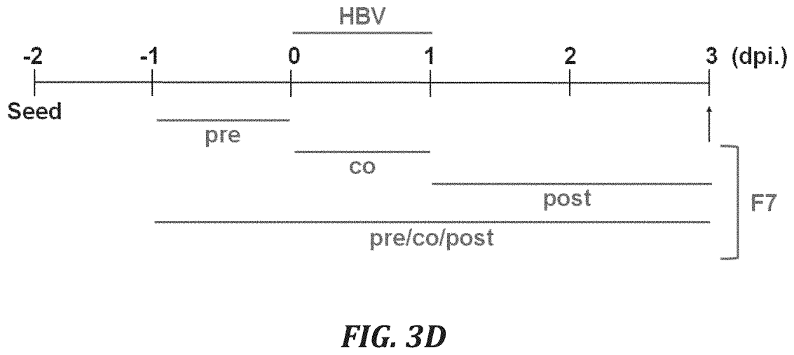

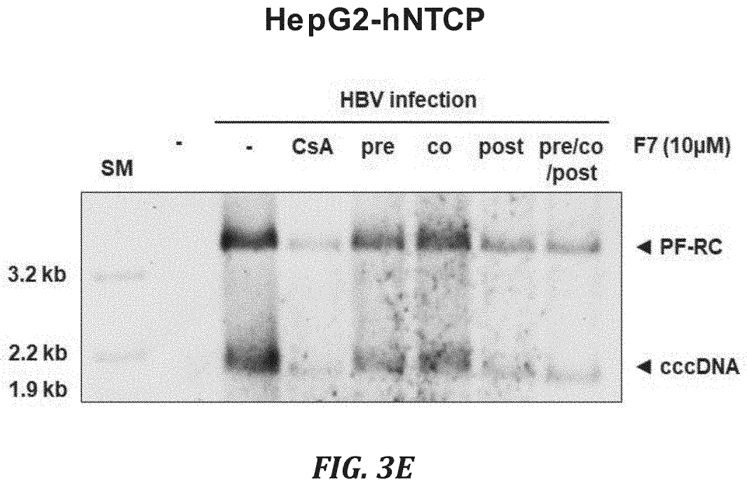

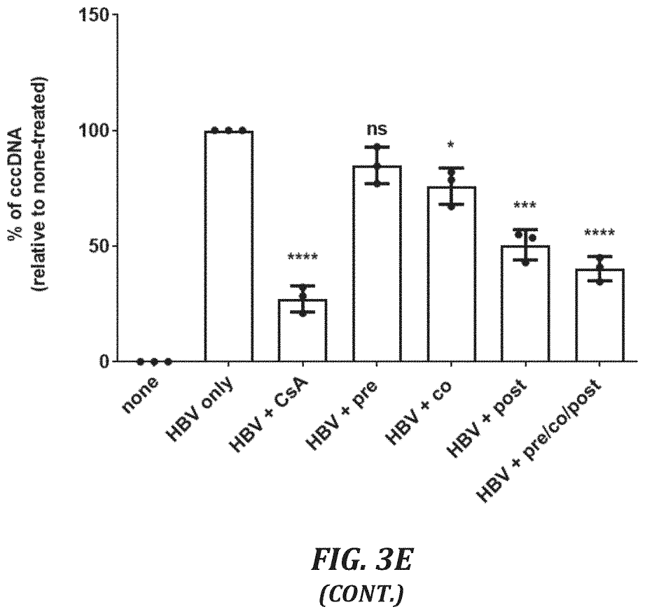

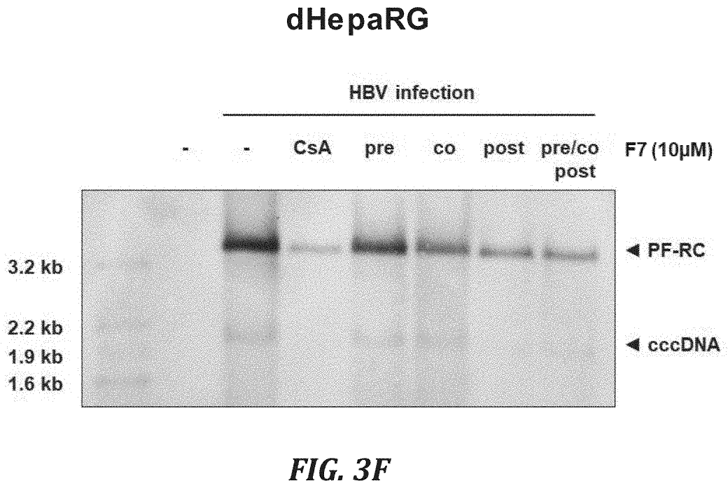

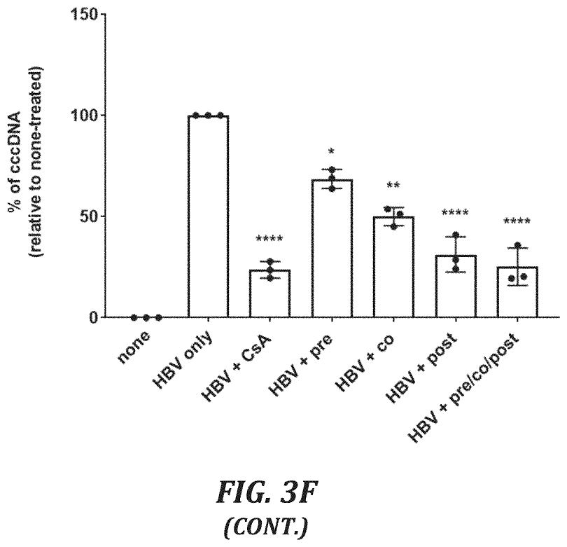

To further define inhibitory effect of F7 and poly-U/UC on cccDNA biosynthesis, time-of-addition experiments were conducted in HepG2-hNTCP and dHepaRG cells. For poly-U/UC PAMP, treatment for 24 hours pre-infection (pre-treatment), from 1 to 3 dpi (post-treatment), and from 24 hours pre-infection onward through 3 dpi (pre/post-treatment) were conducted (FIG. 3A). For F7, treatment for 24 hours pre-infection (pre-treatment), 24 hours initiated at the time of infection (co-treatment), from 1 dpi through 3 dpi (post-treatment), or treatment starting at 24 hours prior to infection and continued through 3 dpi (pre/co/post-treatment) was conducted (FIG. 3D). A co-treatment of CsA with virus inoculation was included as a positive control. Cultures were inoculated with HBV and harvested over the time course for assessment of cccDNA levels using Southern blot and qPCR analyses. Remarkably, it was, found, that the synthesis of cccDNA was uniformly significantly suppressed across the poly-U/UC PAMP regimen, and to a level similar to CsA treatment, in both of HepG2-hNTCP and HepaRG cells (FIGS. 3B and 3C). By comparison, F7 had little effect on HBV cccDNA level when administered once pre or co-treatment but mediated a significant suppression of cccDNA levels when administered post-infection or from pre-infection throughout the 3-day course (FIGS. 3D-3F). These results demonstrate that poly-U/UC PAMP induces an innate immune response that impacts immediate and sustained cccDNA synthesis following HBV infection, while F7 directs an innate immune response that affects cccDNA synthesis post viral entry.

### Antiviral Actions of IRF3 Agonists Partition to the Nucleus to Suppress cccDNA Synthesis

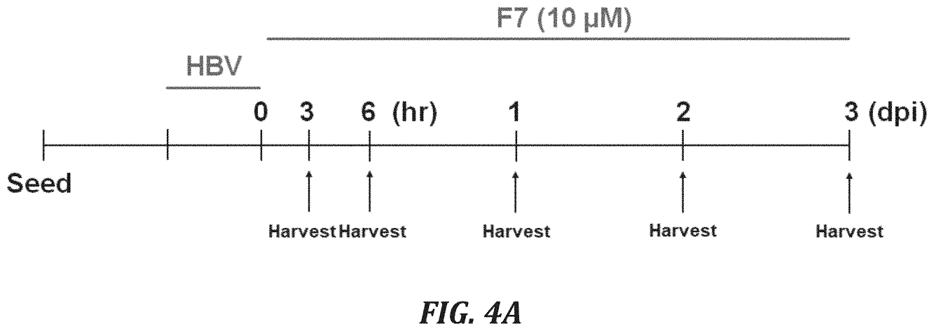

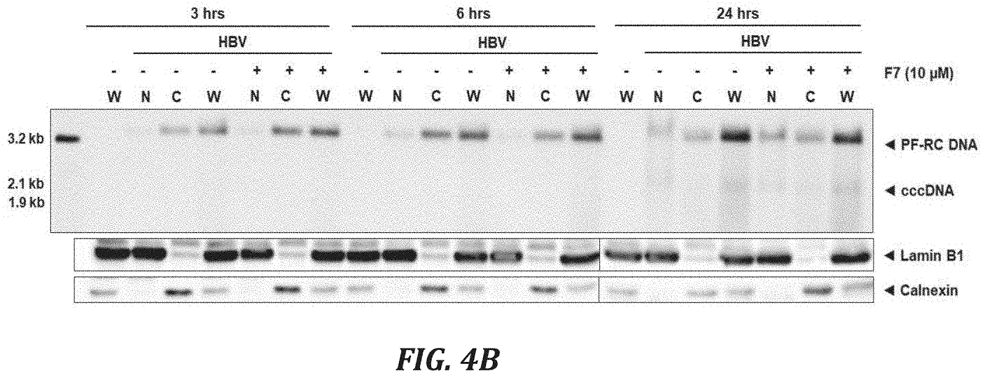

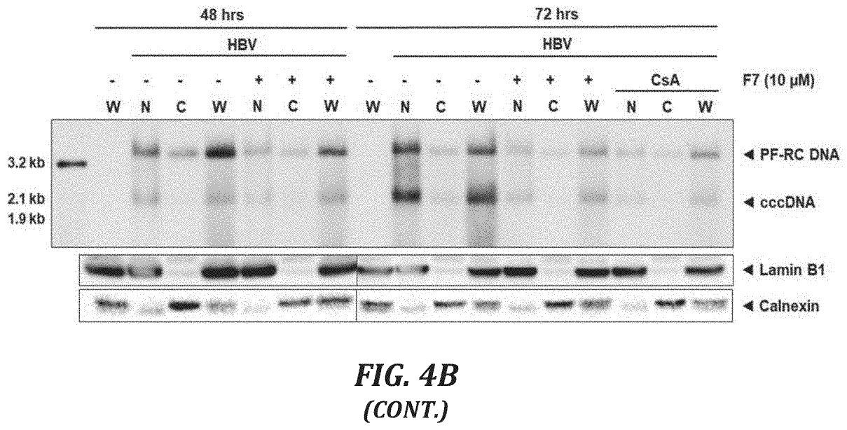

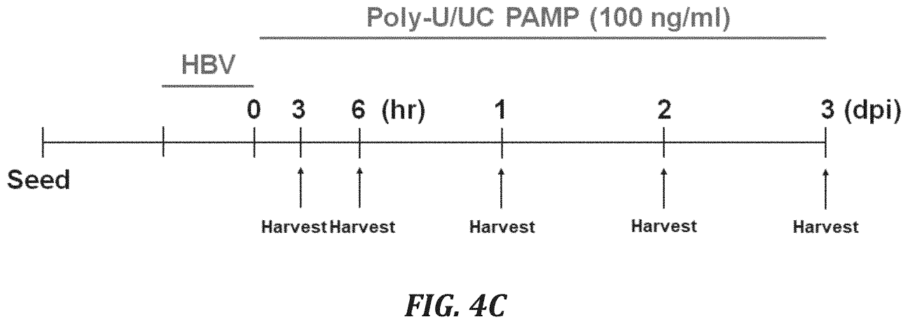

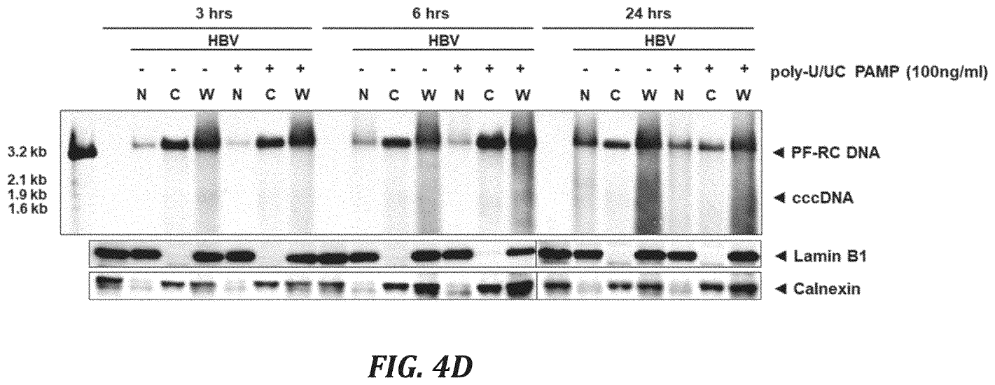

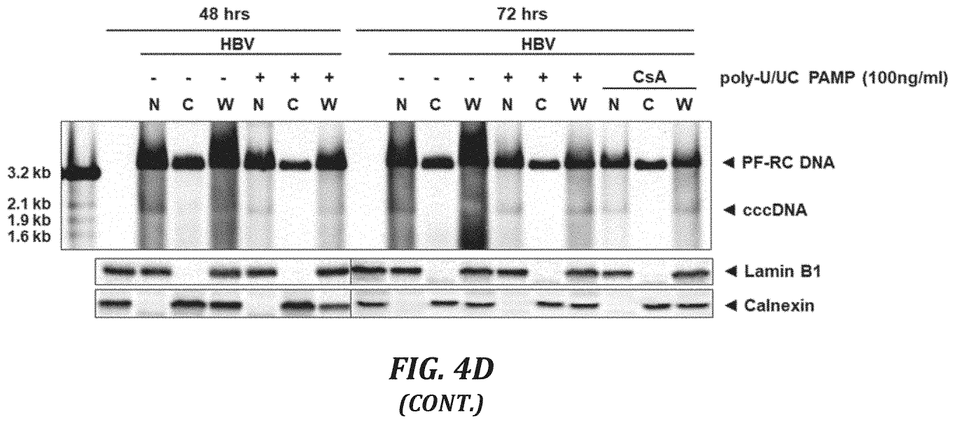

To identify the step(s) of HBV cccDNA synthesis impacted by F7 and poly-U/UC PAMP treatment, the level of cccDNA we assessed over a 3 dpi time course. Cells were inoculated with HBV for 6 hours at 4° C., then the inoculum was removed, cells rinsed and placed in 37° C. media to initiate synchronous infection (time 0), at which time the cells were treated with F7 (FIG. 4A), or poly-U/UC PAMP (FIG. 4C) through 3 dpi. Whole cell (W), nuclear (N), and cytoplasmic (C) extracts were harvested over the time course, at 3 and 6 hours post-infection, and daily over 1-3 dpi, and PF-RC and cccDNA abundance were analyzed by Southern blot. As shown in FIG. 4B, PF-RC DNA was detected in cytoplasmic and whole cell lysate fractions from 3 hours post-infection (hpi) and in nuclear fraction by 6 hpi but accumulation was reduced after 2-3 dpi from F7 treatment. cccDNA was detected by 1 dpi in non-treated cells but cccDNA accumulation was delayed and reduced in cells treated with F7. For poly-U/UC PAMP treatment, cells were also harvested over a similar time course (see FIG. 4C). Assessment of HBV DNA in subcellular fractions also showed that PF-RC DNA was present early in non-treatment cells at 3 and 6 hpi in the whole cell and cytoplasmic extracts with levels accumulating in the nuclear fraction thereafter. Nuclear PF-RC DNA levels were reduced in cells treated with poly-U/UC PAMP from 1-3 dpi concomitantly with reduction of cccDNA in treated cells (FIG. 4D). These results suggest that the inhibitory effects of F7 and poly-U/UC on HBV cccDNA biosynthesis occurs in the nucleus at 1-3 dpi during treatment to possibly impact the conversion step of PF-RC DNA to cccDNA early in the HBV replication process.

### The IRF3 Activation Restricts the Stability of HBV cccDNA alone and in Combination With ETV

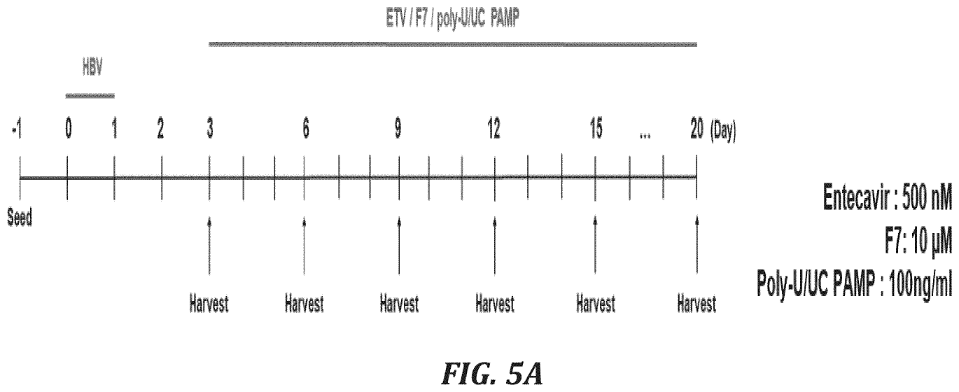

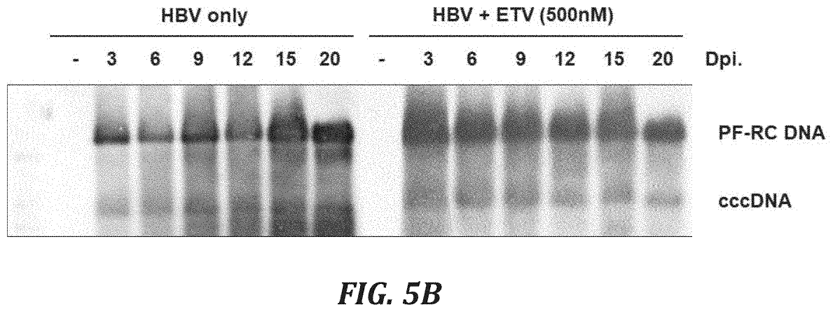

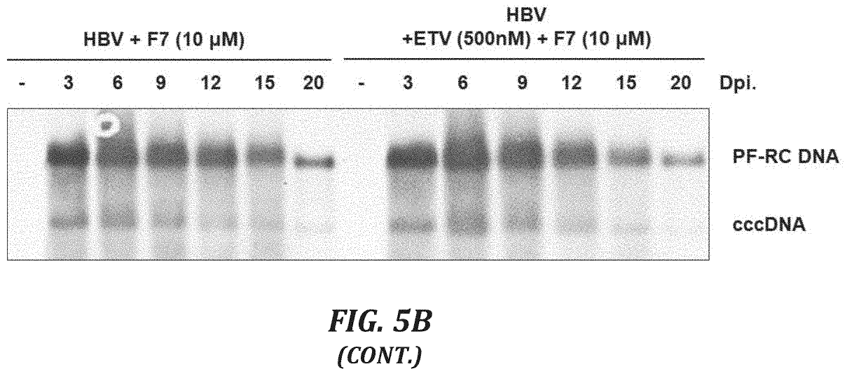

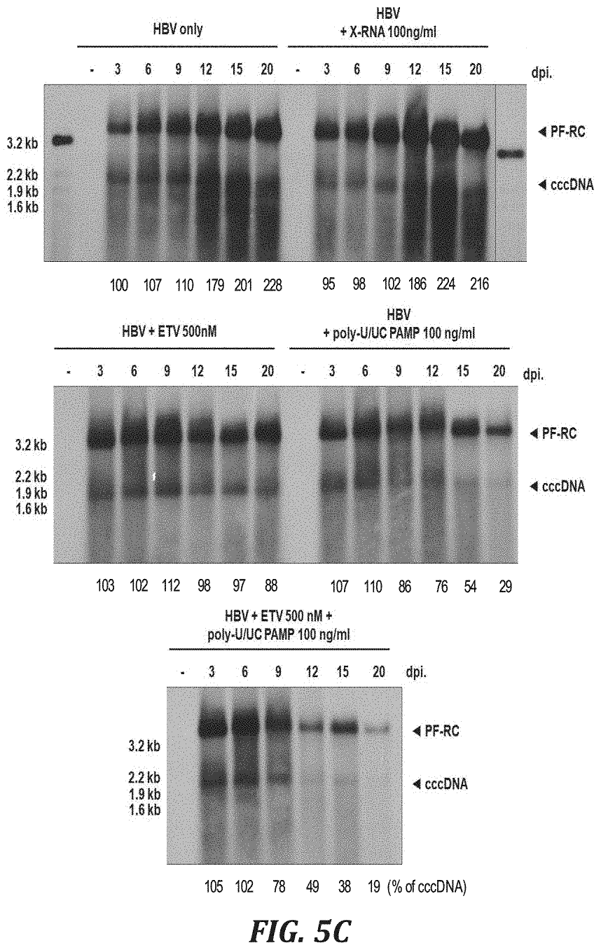

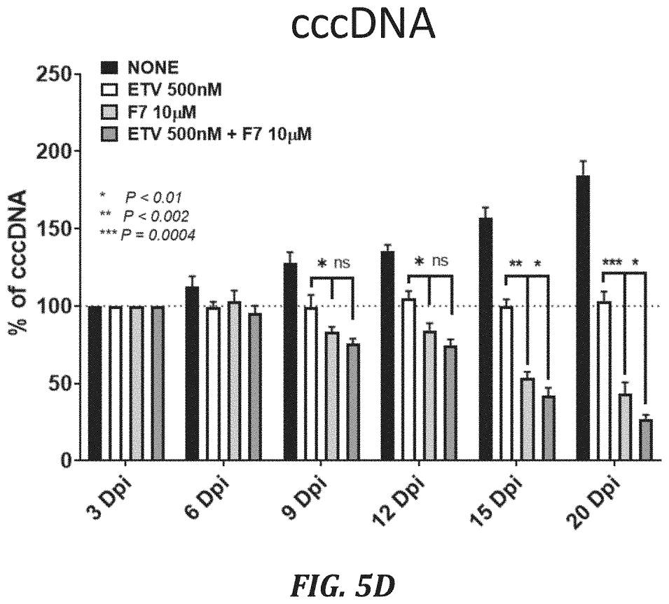

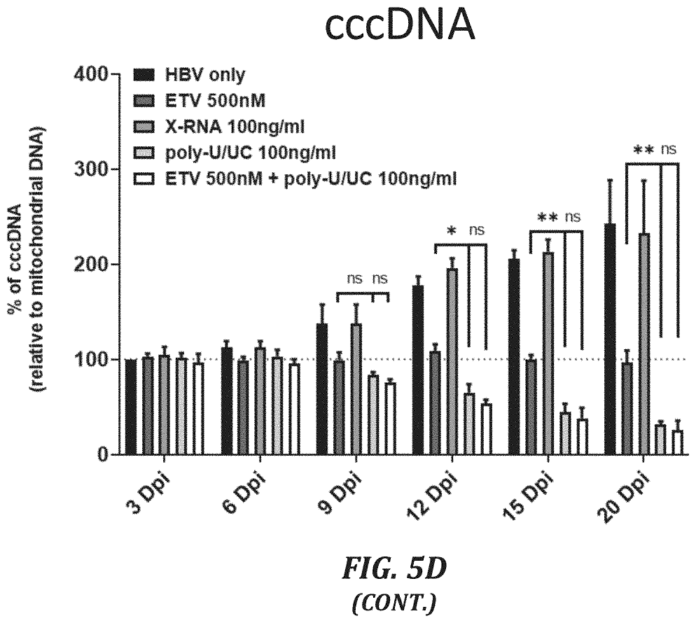

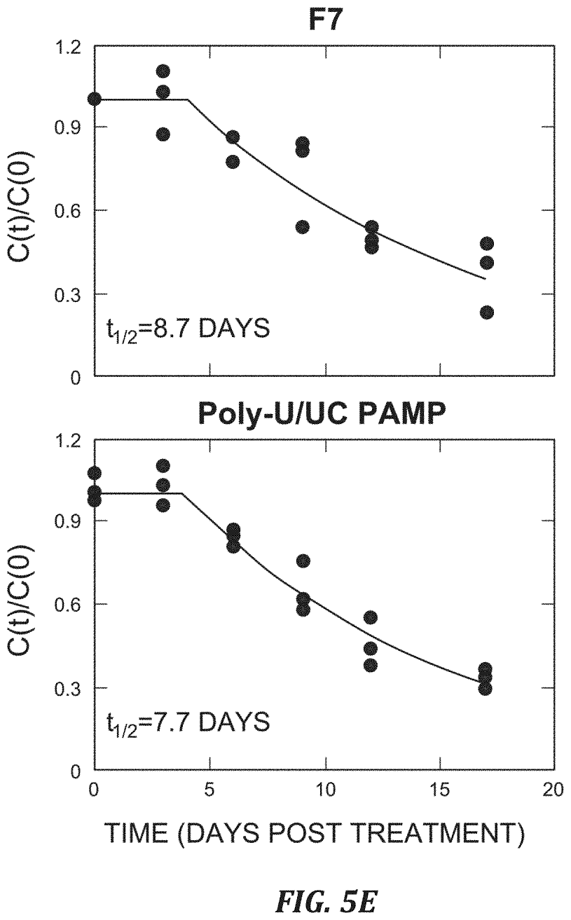

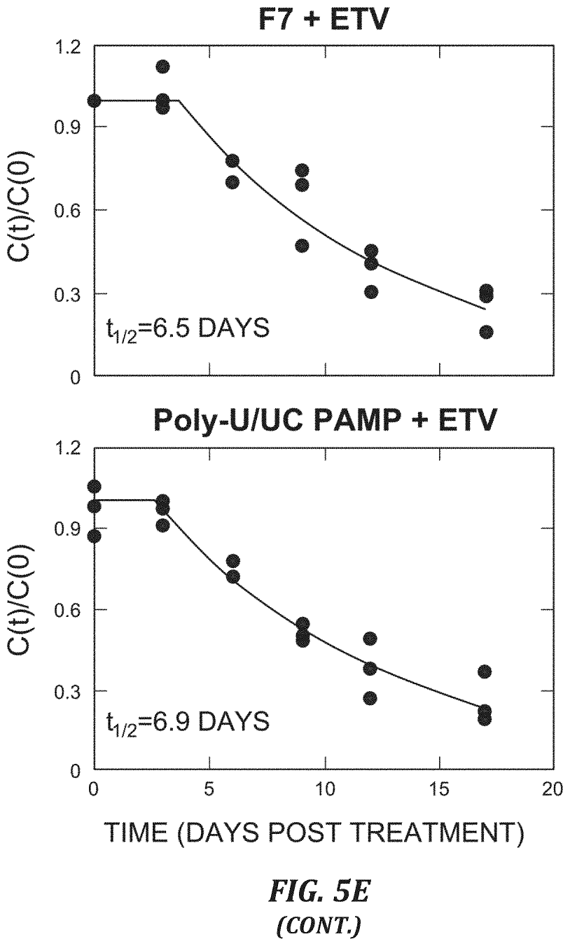

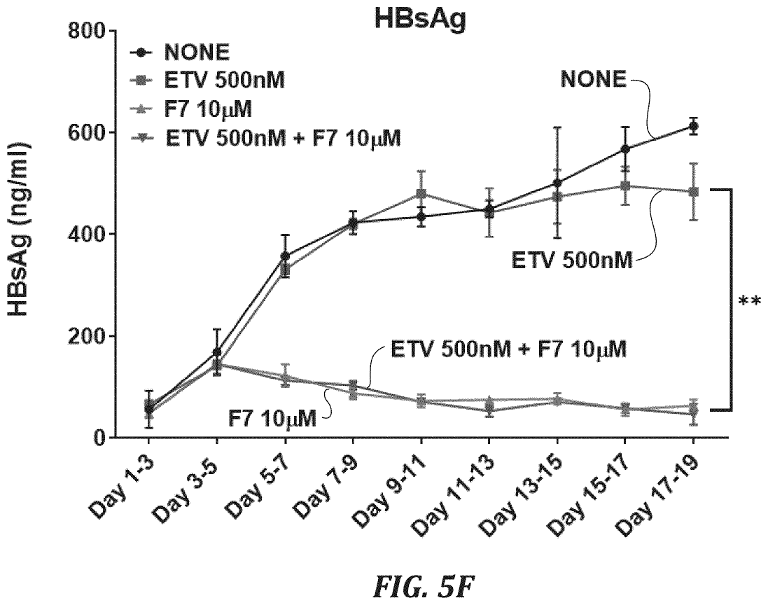

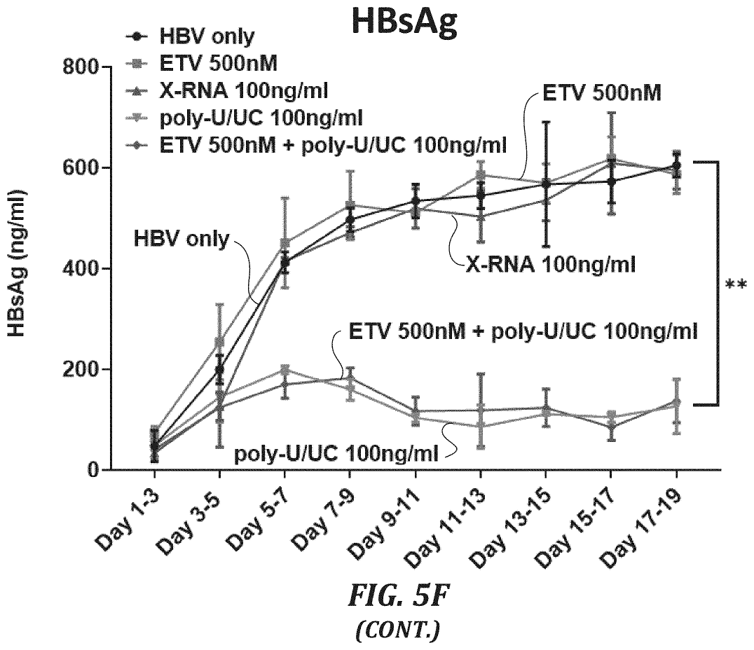

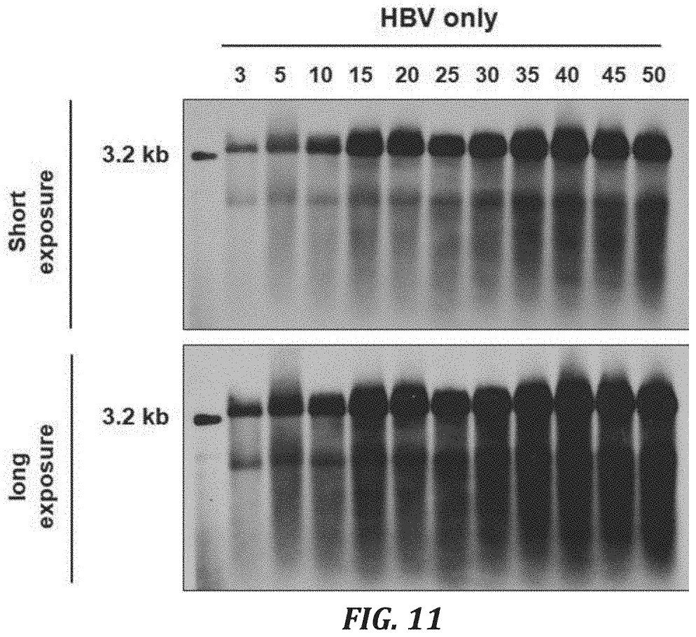

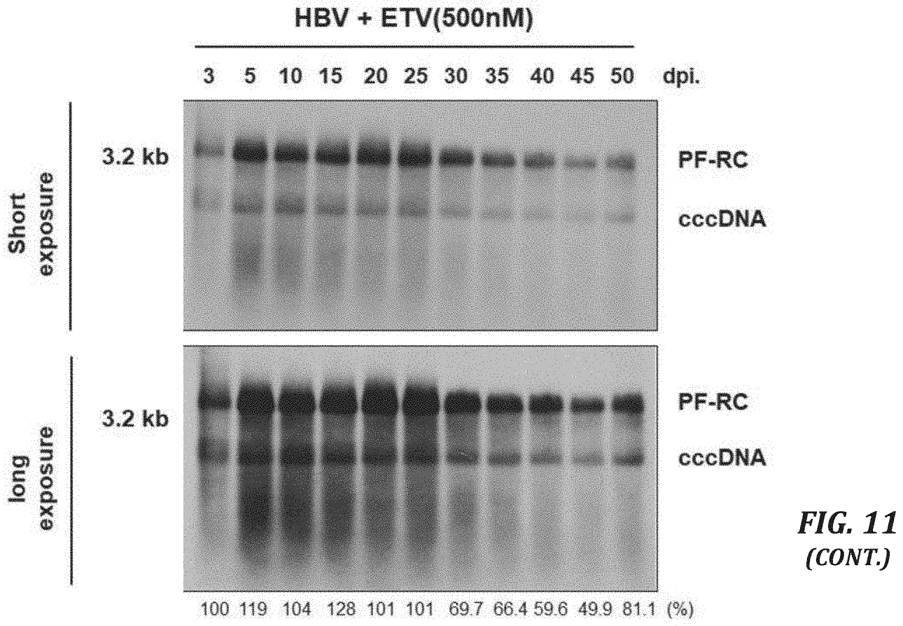

To determine how the host response to IRF3 activation suppresses HBV cccDNA levels, the influence of F7 or poly-U/UC PAMP single treatment and the combination of ETV/F7 or ETV/poly-U/UC PAMP on cccDNA decay kinetics were evaluated. To assess cccDNA decay, F7 and poly-U/UC PAMP treatment of HBV-infected cells we compared with entecavir (ETV) treatment, starting at 3 dpi with treatment maintained through 20 dpi via daily media change with fresh ETV, F7, or poly-U/UC PAMP (FIG. 5A). ETV is a nucleoside analog that prevents the viral reverse transcription and replication of new synthesized HBV DNAs, thereby preventing the replenishment of cccDNA by de novo formation. ETV was applied to HBV-infected cultures at a 100-fold IC₅₀ (Langley, D. R., et al. (2007). Inhibition of Hepatitis B Virus Polymerase by Entecavir. Journal of virology 81, 3992-4001) thereby allowing for the measurement of cccDNA half-life under conditions in which levels are sustained only from the initial cccDNA pool. Cells were then harvested over a treatment time course, and DNA extracts were analyzed by Southern blot and qPCR assay. As shown in FIG. 5B, cccDNA levels modestly increased over the infection time course in non-treatment control cells. Cells from ETV-treated cultures stably maintained cccDNA levels over the time course at levels similar to 3 dpi cultures, demonstrating sustained and stable cccDNA of over 20 dpi in our in vitro culture system, Well in line with the reported cccDNA half-life (t_{1,2}) of greater than 40 days (FIG. 11, (Huang, Q., et (2020). Rapid Turnover of HBV cccDNA Indicated by Monitoring Emergence and Reversion of Signature-Mutation in Treated Chronic Hepatitis B Patients. Hepatology; Ko, C., et al. (2018). Hepatitis B virus genome recycling and de novo secondary infection events maintain stable cccDNA levels. J Hepatol 69, 1231-1241). However, F7 single treatment stimulated the decay of cccDNA as measured by Southern blot analysis (FIG. 5B). Enhanced cccDNA decay kinetics by F7 treatment was also confirmed by RT-PCR analysis (FIG. 5D). F7 mono-treatment reduced the t_{1/2} of cccDNA while the combination of ETV with F7 further enhanced their antiviral effects to reduce cccDNA abundance and persistence to barely detectable level by 20 dpi (FIGS. 5B and 5D). It was also found that a mono-treatment with single administration of poly-U/UC PAMP reduced the cccDNA abundance while it continued to accumulate in cells treated with X RNA control, as analyzed b Southern blot (FIG. 5C) The inhibitory effect on cccDNA levels by poly-U/UC PAMP was also confirmed by q-PCR assay (FIG. 5D) it was also found that poly-U/UC PAMP in combination with ETV served to reduce cccDNA abundance and persistence across a treatment time course over ETV or poly-U/UC PAMP alone, with cccDNA being barely detectable after 20 dpi with cotreatment (FIG. 5C). Mathematical modeling revealed that combination of F7 or poly-U/UC treatment with ETV reduced the cccDNA resulting from monotreatment compared to each respective co-treatment from an average of 8.7 to 6.5 days (F7) and from 7.7 to 6.9 days (poly-U/UC PAMP) (FIG. 5E and TABLE 2), Neither ETV nor X RNA treatment of cells had any effect on the production and secretion of HBsAg. However, poly-U/UC PAMP and F7 mono-, and combination treatments suppressed HBsAg secretion (FIG. 5F). Thus, F7 and poly-U/UC PAMP impart the therapeutic decay of established cccDNA. Moreover, combination treatment of F7 and poly-U/UC PAMP with ETV impart additive antiviral actions to suppress cccDNA t_{1/2} and persistence from weeks (Huang, Q., et al. (2020), supra; Ko, C., et al. (2018), supra) to less than 7 days.

TABLE 2
The half-life of cccDNA, (t_{1/2}) and the delay before
cccDNA starts decreasing (τ) estimated
from the kinetics of decay of cccDNA under treatment.

| Treatment | Parameter values | 95% Confidence Interval |
|---|---|---|
| F7 | | |
| t_{1/2} (days) | 8.7 | [7.2, 14.0] |
| τ (days) | 4.0 | [1.4, 6.6] |
| F7 + ETV | | |
| t_{1/2} (days) | 6.5 | [5.5, 8.5] |
| τ (days) | 3.7 | [1.6, 5.8] |
| Poly-U/UC PAMP | | |
| t_{1/2} (days) | 7.7 | [7.4, 9.8] |
| τ (days) | 3.8 | [2.4, 5.2] |
| Poly-U/UC PAMP + ETV | | |
| t_{1/2} (days) | 6.9 | [5.8, 10.5] |
| τ (days) | 2.6 | [1.0, 4.3] |

Suppression of HBV cccDNA by IRF3 activation is RIG-I-dependent

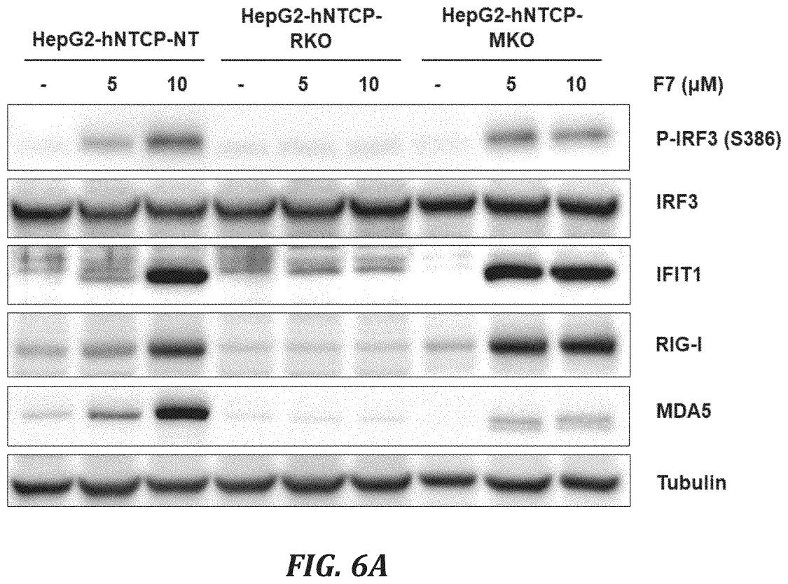

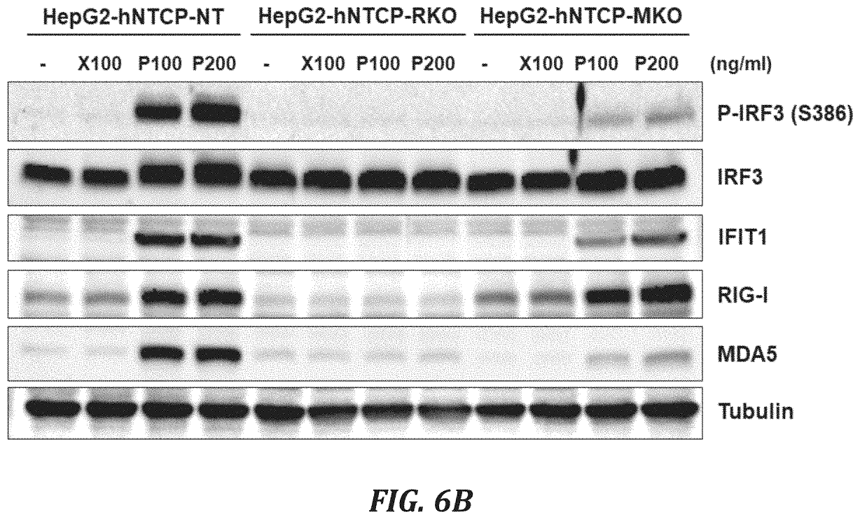

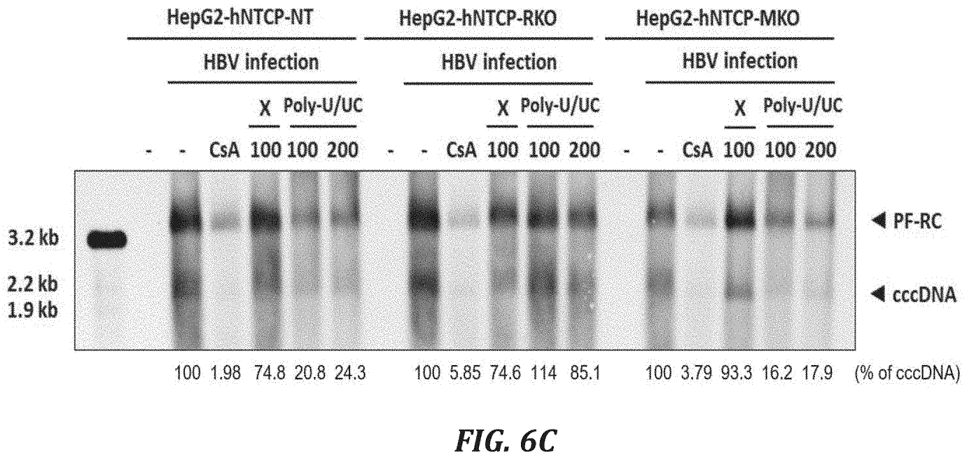

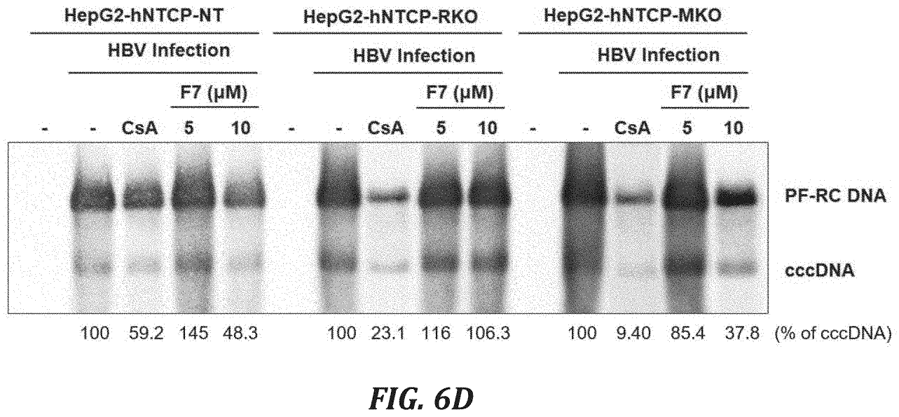

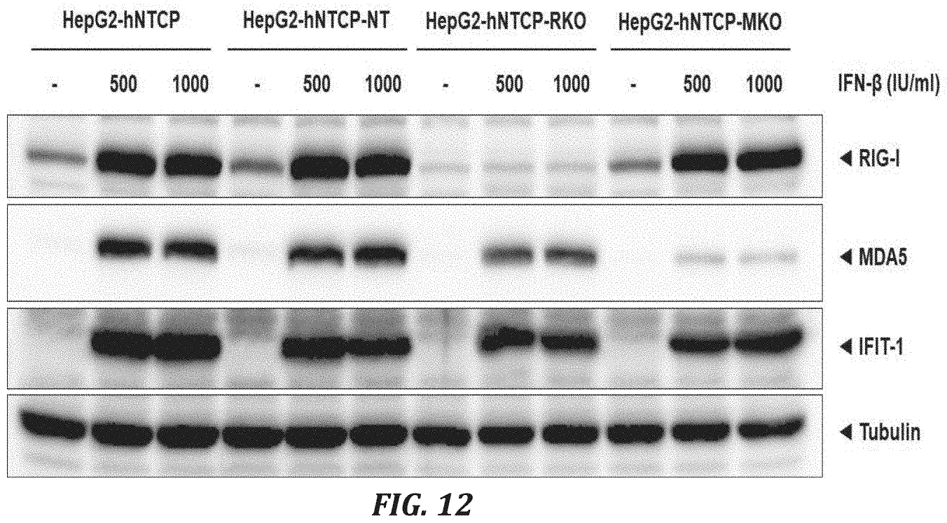

In order to ascertain whether the activation of IRF3 and suppression of cccDNA in HBV-infected cells by F7 or poly-U/UC PAMP treatment was dependent on RIG-I rather than occurring as an off-target effect of IRF3 agonist treatment, HepG2-hNTCP cells expressing non-targeting guide RNA (HepG2-hNTCP-NT), or guide RNA for knockout (KO) of RIG-I expression (HepG2-hNTCP-RKO) or MDA5 (HepG2-hNTCP-MKO) were produced using CRISPR/Cas9 genome editing technology. Immunoblot analysis of the different cell populations shows that HepG2-hNTCP-RKO or HepG2-hNTCP-MKO cells do not express detectable levels of RIG-I or MDA5, respectively (FIG. 12). Treatment of cells with F7 (FIG. 6A) or poly-U/UC PAMP (FIG. 6B) shows that the HepG2-hNTCP-RKO cells do not respond to treatment while HepG2-hNTCP-NT control cells and HepG2-hNTCP-MKO cells fully respond to F7 to accumulate phosphorylated/activated IRF3 concomitant with expression of IFIT1. HepG2-hNTCP-MKO cells serve as an RLR KO control to reveal the specificity of F7 and poly-U/UC PAMP for triggering RIG-I-dependent IRF3 activation (Saito et al. (2008), supra). Next, each cell population was infected with HBV followed by a single treatment with F7 or poly-U/UC PAMP at 1 dpi. Cells were harvested at 3 dpi for Southern blot analysis of cccDNA levels. Parallel cultures of each cell population were treated with DMSO or single dose X-RNA (negative control) or with CsA as a treatment control. F7 treatment reduced cccDNA levels in HepG2-hNTCP-NT and HepG2-hNTCP-MKO cells but not in HepG2-hNTCP-RKO cells (FIG. 6C). Similarly, poly-U/UC PAMP suppression of cccDNA was dependent of RIG-I, as cccDNA was suppressed by poly-U/UC PAMP treatment in HepG2-hNTCP-NT and HepG2-hNTCP-MKO cells but not in the HepG2-hNTCP-RKO cells (FIG. 6D). These results demonstrate that the antiviral actions of F7 and poly-U/UC PAMP against HBV specifically signal through RIG-I, defining each as a RIG-I agonist that activates IRF3 to impart suppression of cccDNA.

### RIG-I Signaling of IRF3 Activation Suppresses HBV Infection in Primary Human Hepatocytes

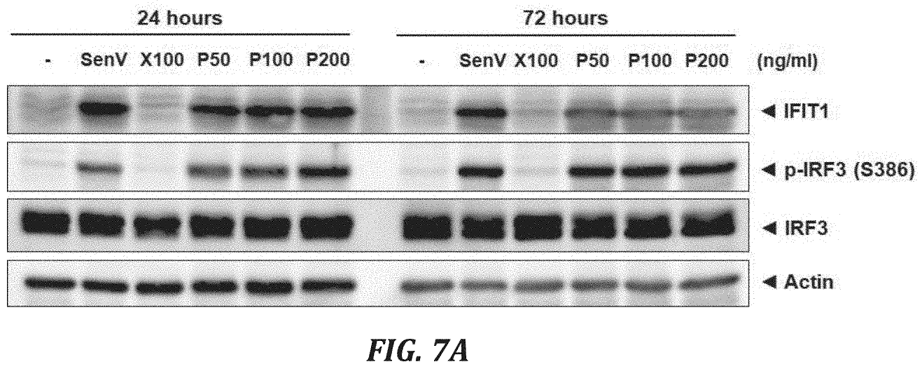

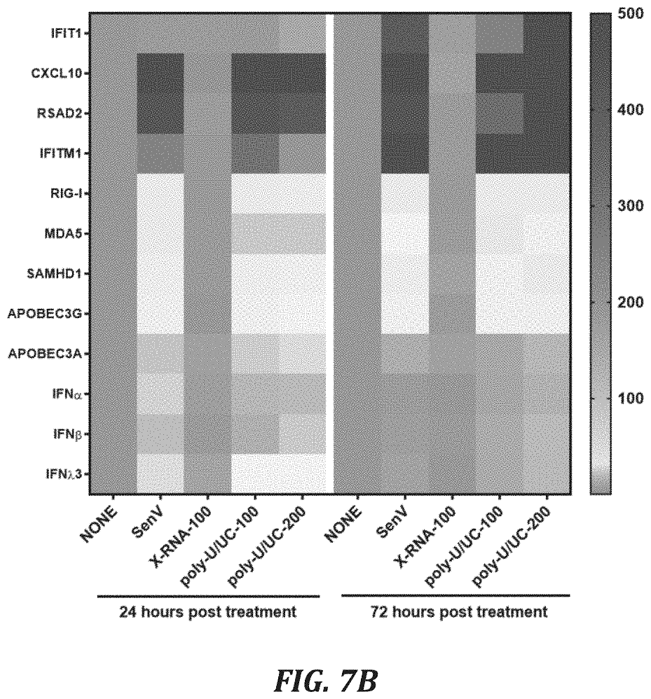

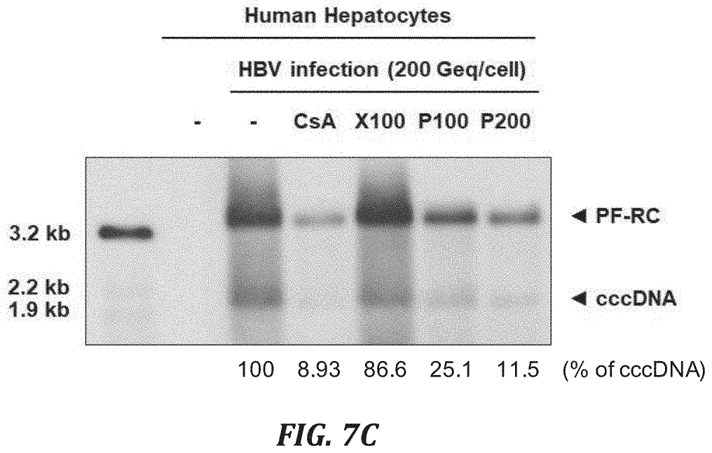

To validate the antiviral actions of RIG-I signaling of IRF3 activation by poly-U/UC PAMP, HBV infection were assessed in non-immortalized and terminally differentiated primary human hepatocytes (PHH) that retain the expression of hepatocyte marker genes at a level comparable to that of human liver tissue. PHH cultures were treated over a poly-U/UC PAMP dose-response and assessed innate immune activation. Treatment of PHH cultures with 50-200 ng/ml of poly-U/UC PAMP induced IRF3 activation marked by accumulation of phosphoserine 386. IRF3 and IFIT1 expression. Treatment with 100 ng/ml X-RNA did not induce innate immune activation of PHH but cells were fully responsive to acute infection with SenV control (FIG. 7A), demonstrating that PHHs harbor an intact RIG-I pathway. Moreover, PHH treatment with poly-U/UC PAMP but not X-RNA robustly induced innate immune gene expression (FIG. 7B). cccDNA levels were then evaluated in HBV-infected PHH treated with poly-U/UT PAMP. PHH cultures were infected with HBV, and subject to a single treatment with poly-U/UC PAMP at 1 dpi. Cells were harvested at 3 dpi, DNA extracted and subject to Southern blot analysis. As shown in FIG. 7C, treatment with poly-U/UC PAMP suppressed cccDNA levels in the infected PHH to levels similar to CsA treatment. Thus, poly-U/UC treatment to trigger RIG-I signaling of IRF3 and innate immune activation directs a response in HBV-infected PHH that suppresses cccDNA. These results demonstrate the efficacy of therapeutic targeting the RIG-I pathway for viral control in relevant primary cells of HBV infection.

### Discussion

Persistence of cccDNA in the nucleus of HBV-infected hepatocytes is key to mediating chronic HBV infection, wherein recent analyses indicates that a given pool cccDNA has a t_{1/2} in vivo of approximately 5-21 weeks (Huang, Q., et al. (2020), supra). Problematically, current therapies for the treatment of chronic HBV infection neither significantly reduce nor eliminate cccDNA (Maynard, M., et al. (2005). Sustained HBs seroconversion during lamivudine and adefovir dipivoxil combination therapy for lamivudine failure. J Hepatol 42, 279-281; Werle-Lapostolle, et al. (2004). Persistence of cccDNA during the natural history of chronic hepatitis B and decline during adefovir dipivoxil therapy, Gastroenterology 126, 1750-1758; Zoulim, F., and Durantel, D. (2015). Antiviral therapies and prospects for a cure a chronic hepatitis B. Cold Spring Harb Perspect Med 5). Members of the current nucleoside analogs class of HBV therapeutics are administered for prolonged, often lifelong periods to keep patients virally suppressed. Moreover, these therapies are leaky in their ability to completely shut down viral replication, such that the nuclear cccDNA pool (Huang, Q., et al. (2020), supra) still persists after long-term treatment (Gish, R., et al. (2012). Selection of chronic hepatitis B therapy with high barrier to resistance. Lancet Infect Dis 12, 341-353: Werle-Lapostolle, et al. (2004), supra; Zoulim, F., and Locarnini, S. (2009). Hepatitis B virus resistance to nucleos(t)ide analogues. Gastroenterology 137, 1593-1608.e1591-1592), Importantly however, it has been demonstrated that suppression of cccDNA during acute HBV infection can occur through a cytokine-driven non-cytolytic mechanism directed by IFN-α, IFN-β, or tumor necrosis factor-α linked with expression of APOBEC3 deaminases (Lucifora et al. (2014) supra; Xia, Y., et al. (2016). Interferon-gamma and Tumor Necrosis Factor-alpha Produced by T Cells Reduce the HBV Persistence Form, cccDNA, Without Cytolysis. Gastroenterology 150, 194-205). Pharmacological induction of intrahepatic cytokine responses has been coined as an ideal curative approach to chronic hepatitis B (Chang, J., et al. (2012). The innate immune response to hepatitis B virus infection: implications for pathogenesis and therapy. Antiviral research 96, 405-413). While this approach leverages the innate immune and immune modulatory signaling programs driven by specific cytokines to induce the expression of genes whose actions can suppress cccDNA, cytokine therapy for treatment of chronic HBV faces the obstacles of systemic toxicity from the broad off target, nonhepatic actions of cytokine treatment (Kwon, H., and Lok, A. S. (2011), Hepatitis B therapy. Nature reviews Gastroenterology & hepatology 8, 275-284; Locarnini, S., et al. (2015). Strategies to control hepatitis B: Public policy, epidemiology, vaccine and drugs. Journal of hepatology 62, S76-86), underscoring the need for target-directed therapy strategies that can also be coupled with current NA therapeutics against HBV.

It is demonstrated here that targeting RIG-I and the RLR pathway to activate IRF3, using the F7 small molecule and poly-U/UC PAMP agonists of RIG-I as proof of concept, delivered to hepatocytes directs a specific RIG-I-dependent innate immune response through IRF3 that effectively suppresses HBV cccDNA in a cell culture model of HBV infection. Further, mechanistic studies show that the administration of F7 or poly-U/UC PAMP suppresses de novo biosynthesis of cccDNA and enhanced cccDNA degradation rather than inhibiting the production of newly synthesized HBV rcDNA as current NA HBV drugs do. Administration of F7 and poly-U/UC PAMP induces IRF3 activation and expression of IRF3-target genes (FIGS. 1A-1D). Thus, the underlying mechanisms of F7 and poly-U/UC PAMP to suppress cccDNA likely involve the actions of IRF3 target genes to block cccDNA synthesis and impart actions that facilitate the destabilization and degradation of cccDNA to deplete it from the infected cell. It is noted that F7 treatment of cells does not induce expression of type I or III IFN, owing to the RIG-I-activation properties of this class of compounds that do not impart signaling to NF-kB but instead exclusively activate downstream IRF3 (Bedard et al. (2012), supra; Probst, et al. (2017), supra). Thus, in the case of F7 the antiviral actions to suppress cccDNA operate independent of IFN actions but through IRF3-responsive genes. These results show that poly-U/UC PAMP also induces robust IRF3-target gene expression as well as a low level of IFN that can induce ISGs. Among these are the APOBEC3 genes, known antiviral effectors against HBV replication (Bonvin, et al. (2006). Interferon-inducible expression of APOBEC3 editing enzymes in human hepatocytes and inhibition of hepatitis B virus replication. Hepatology (Baltimore, Md.) 43, 1364-1374; Turelli, P., et al. (2004). Inhibition of Hepatitis B Virus Replication by APOBEC3G. Science 303, 1829). IRF3 activation drives the expression and production of various immune modulatory cytokines and chemokines, including CXCL10, a chemoattractant for T cells (Sankar, S., et al. (2006). IKK-i signals through IRE; and NFkappaB to mediate the production of inflammatory cytokines. Cell Signal 18, 982-993; Zhai, Y., et al. (2008). CXCL10 regulates liver innate immune response against ischernia and reperfusion injury. Hepatology 47, 207-214), as well as directs the expression of factors that regulate ubiquitination (Maelfait, J., and Beyaert, R. (2012). Emerging role of ubiquitination in antiviral RIG-I signaling. Microbiol Mol Biol Rev 76, 33-45) and a variety of cell signaling process (Zhou, Y., et al. (2017), Post-translational regulation of antiviral innate signaling. Eur J Immunol 47, 1414-1426). Thus IRF3-target genes are proposed to include a variety of anti-cccDNA effectors in addition to the known actions of the APOBEC genes. These effector genes then impart pleiotropic actions to i) suppress cccDNA amplification, and ii) destabilize cccDNA and/or enhance cccDNA degradation. Indeed; a marked reduction in the cccDNA t_{1/2} was observed when cells were treated with F7 or poly-U/UC PAMP, and this reduction was further enhanced when cells were cotreated with either of these plus entecavir. In addition, for poly-U/UC PAMP, the antiviral actions against cccDNA could also include IFN-mediated actions directed by the low level IFN induction (Isorce, N., et al. (2016). Antiviral activity of various interferons and pro-inflammatory cytokines in non-transformed cultured hepatocytes infected with hepatitis B virus. Antiviral research 130, 36-45; Lucifora, J., et al. (2014). supra. Phillips, S., et al. (2017). Peg-Interferon Lambda Treatment Induces Robust Innate and Adaptive immunity in Chronic Hepatitis B Patients. Frontiers in Immunology 8; Robek, M. D., et al. (2005). Lambda Interferon Inhibits Hepatitis B and C Virus Replication. Journal of Virology 79, 38.51-3854; Xu, F., et at. (2018). Type III interferon-induced CBFβ inhibits HBV replication by hijacking HBx. Cellular & Molecular Immunology). Interestingly, these actions of IFN and specific ISGs resulting from treatment with poly-U/UC PAMP could have contributed to a suppression of cccDNA resulting in a modestly shorter half-life compared to treatment with F7. Mechanistically, it is proposed that the antiviral action of F7 and poly-U/UC PAMP might also include alteration of the cellular DNA repair machinery that otherwise contribute to cccDNA biosynthesis. As HBV takes advantage of host DNA repair factors to repair the discontinuity of RC-DNA and convert it into a transcription permissive cccDNA, several cellular DNA repair proteins known to be involved in cccDNA metabolism, including TDP2 (Koniger, C., et al. (2014). Involvement of the host DNA-repair enzyme TDP2 in formation of the covalently closed circular DNA persistence reservoir of hepatitis B viruses. Proceedings of the National Academy of Sciences of the United States of America 111, E4244-4253), DNA ligases (Long, Q., et at (2017). The role of host DNA ligases in hepadnavirus covalently closed circular DNA formation, PLoS pathogens 13, e1006784), DNA topoisomerase (Sheraz, M., et al. (2019). Cellular DNA Topoisomerases Are Required for the Synthesis of Hepatitis B Virus Covalently Closed Circular DNA. Journal of virology 93), and DNA polymerase (pol α, λ, κ) (Qi. Y., et al. (2016). DNA Polymerase kappa. Is a Key Cellular Factor for the Formation of Covalently Closed Circular DNA of Hepatitis B Virus. PLoS pathogens 12, e1005893; Tang, L., et al. (2019). DNA Polymerase alpha is essential for intracellular amplification of hepatitis B virus covalently closed circular DNA. PLoS pathogens 15, e1007742) could be candidates regulated by RIG-I/IRF3 signaling.

The present studies demonstrate that RIG-I and IRF3 can be specifically targeted to activate the RLR innate immune program for the control of HBV infection through suppression of cccDNA, reducing the t_{1/2} to days compared to weeks or months in the absence of treatment. Targeting innate immunity and the RLR pathway thus offers an effective strategy toward new antiviral therapies against HBV that can be offered alone or in combination with NA for HBV treatment. Determining the innate immune targets directed by RIG-I and IRF3 that impart depletion of cccDNA will bring insight into the mechanism of action and unique antiviral properties of these novel drug candidates toward an HBV cure.

### Methods and Materials

TABLE 3
PCR resources.

| No | Oligo name | Sequence (5′→3′) (SEQ ID NO: in parentheses) |
|---|---|---|
| 1 | HBV cccDNA Fwd | GCCTATTGATTGGAAAGTATGT (2) |
| 2 | HBV cccDNA Rev | GCTGAGGCGGTATCTA (3) |
| 3 | HBV pgRNA Fwd | CTCCTCCAGCTTATAGACC (4) |
| 4 | HBV pgRNA Rev | GTGAGTGGGCCTACAAA (5) |
| 5 | IFIT1 Fwd | AGAAGCAGGCAATCACAGAAAA (6) |
| 6 | IFIT1 Rev | CTGAAACCGACCATAGTGGAAAT (7) |
| 7 | IFITM1 Fwd | TACTCCGTGAAGTCTAGGGACAG (8) |
| 8 | IFITM1 Rev | AACAGGATGAATCCAATGGTCA (9) |
| 9 | CXCL10 Fwd | GTGGCATTCAAGGAGTACCTC (10) |
| 10 | CXCL10 Rev | TGATGGCCTTCGATTCTGGATT (11) |
| 11 | RSAD2 Fwd | CGTGAGCATCGTGAGCAATG (12) |
| 12 | RSAD2 Rev | TCTTCTTTCCTTGGCCACGG (13) |
| 13 | RIG-I Fwd | Qiagen SABiosciences #PPH20774A |
| 14 | RIG-I Rev | |
| 15 | MDA5 Fwd | Qiagen SABiosciences #PPH18927A |
| 16 | MDA5 Rev | |
| 17 | IFNα Fwd | TGCACCGAACTCTACCAGCA (14) |
| 18 | IFNα Rev | GTTTCTCCCACCCTCTCCTCC (15) |
| 19 | IFNβ Fwd | Qiagene SABiosciences # PPH00384F |
| 20 | IFNβ Rev | |
| 21 | IFNγ Fwd | Qiagene SABiosciences # PPH00380C |
| 22 | IFNγ Rev | |
| 23 | IFNλ3 Fwd | AAGGACTGCAAGTGCCGCT (16) |
| 24 | IFNλ3 Rev | GCTGGTCCAAGACATCCC (17) |
| 25 | SAMHD1 Fwd | TCACAGGCGCATTACTGCC (18) |
| 26 | SAMHD1-Rev | GGATTTGAACCAATCGCTGGA (19) |
| 27 | APOBEC3A Fwd | GAGAAGGGACAAGCACATGG (20) |
| 28 | APOBEC3A Rev | TGGATCCATCAAGTGTCTGG (21) |
| 29 | APOBEC3G Fwd | CCCTAGGACCCGAACTGTTAC (22) |
| 30 | APOBEC3G Rev | TCCAACAGTGCTGAAATTCG (23) |
| 31 | GAPDH Fwd | ACAACTTTGGTATCGTGGAAGG (24) |
| 32 | GAPDH Rev | GCCATCACGCCACAGTTTC (25) |
| 33 | MT-CO3* Fwd | CCCCACAAACCCCATTACTAAACCCA (26) |
| 34 | MT-CO3* Rev | TTTCATCATGCGGAGATGTTGGATGG (27) |

*MT-CO3: mhochondrial cytochrome c oxidase subunit 3

### Experimental Model and Subject Details

Cell cultures: The human NTCP stably expressing human hepatoma line C3A, a subclone of HepG2 were maintained in Dulbecco modified Eagle medium (DMEM) supplemented with 10% heat-inactivated FBS, 1× Glutamax (GIBCO), 100 U/ml penicillin, and 100 μg/ml streptomycin and were selected/expanded with medium containing 1 μg/ml of puromycin as previously described (Guo, F., et al. (2017). HBV core protein allosteric modulators differentially alter cccDNA biosynthesis from de novo infection and intracellular amplification pathways. PLoS pathogens 13, e1006658; Ko, C., et al. (2014a). DDX3 DEAD-Box RNA Helicase Is a Host Factor That Restricts Hepatitis B Virus Replication at the Transcriptional Level. Journal of virology 88, 13689-13698). HepAD38 cell line, which support produce HBV in tetracycline (TET)-inducible manner, were maintained as previously described (Watashi, K., et al. (2013). Interleukin-1 and tumor necrosis factor-alpha trigger restriction of hepatitis B virus infection via a cytidine deaminase activation-induced cytidine deaminase (AID). The Journal of Biological Chemistry 288, 31715-31727). The human liver progenitor HepaRG cell line was cultured in complete Williams E medium supplemented with 10% FBS, 100 U/ml penicillin, 100 μg/ml streptomycin, Hydrocortisone 21-Hemisuccinate (Cayman), human insulin (Sigma), and 1× Glutamax (GIBCO) (Gripon, P., et al. (2002). Infection of a human hepatoma cell line by hepatitis B virus. Proceedings of the National Academy of Sciences of the United States of America 99 15655-15660). Primary human hepatocytes were freshly isolated from chimeric mice that have humanized liver reconstituted with PHH. The recovered PHH were cultured in DMEM supplemented with 10% heat-inactivated FBS, 15 μg/ml L-proline, 25 ng/ml insulin, 50 μM Dexamethasone, 5 ng/ml EGF, and 0.1 mM L-ascorbic acid 2-phospate, as described previously (Ishida, Y., et al. (2015). Novel robust in vitro hepatitis B virus infection model using fresh human hepatocytes isolated from humanized mice. The American journal of pathology 185, 1275-1285).

Generation of HepG2-hNTCP-NT/RIG-I/MDA5 KO cell lines by using CRISPR system: For expression of human sodium taurocholate co-transporting polypeptide (hNTCP), the gene coding sequence was amplified from a cDNA clone prepared from dHepaRG cells. A carboxyl-terminal C9 tag was added by PCR amplification. Transduced cells were selected with 20 μg/ml blasticidin and the best growing single cell clones were screened for their ability to support HBV infection. For CRISPR/Cas mediated gene knockout, guide RNA (gRNA) sequences were designed with the CRISPR tool of Benefiting (Biology Software, 2017, https://benchling.com). The gRNA target oligonucleotides were cloned into Cas9-t2a-pRRL lentiviral vector by using the In-Fusion cloning kit (Takara). gRNA sequences used for gene knockouts were gRIG-I: 5′-GGGTCTTCCGGATATAATCC-3′(SEQ ID NO:28), and gMDA5: 5′-GTGGTTGGACTCGGGAATTCG-3′(SEQ ID NO:29) (Esser-obis; K., et al. (2019). Comparative Analysis of African and Asian Lineage-Derived Zika Virus Strains Reveals Differences in Activation of and Sensitivity to Antiviral Innate Immunity. Journal of virology 93). Upon transduction, cells were kept under continuous selection with 10 μg/ml puromycin and knockouts were confirmed by western blot.

### Method Details

HBV infection HBV (Genotype D) was purified from the supernatant of HepAD38 cells by PEG concentration and subsequent sucrose gradient, as described previously (Ko, C., et al. (2014). DDX3 DEAD-Box RNA Helicase Is a Host Factor That Restricts Hepatitis B Virus Replication at the Transcriptional Level. Journal of Virology 88, 13689-13698; Watashi, K., et al. (2013), supra). For HBV infection cells were seeded into collagen-coated plates. One day later, the cells were infected with HBV in DMEM containing 4% polyethylene glycol 8000 (PEG-8000). The multiplicities of infection (expressed as virus genome equivalent/cell) are indicated in each Figure Legend. The inocula were removed 24 hours later, and the infected cultures were maintained in complete DMEM containing 2.5% DMSO until harvesting, as described previously (Ni, Y., et al. (2014). Hepatitis B and D viruses exploit sodium taurocholate co-transporting polypeptide for species-specific entry into hepatocytes. Gastroenterology 146, 1070-1083).

Reagents: F7 is a small molecule based on a benzothiazol core structure identified in a high-throughput screen for IRF3 agonists (U.S. Pat. No. 9,884,876, Probst, et al. (2017), supra). F7 (N-(6-benzamido-1,3-benzothiazol-2-yl)naphthalene-2-carboxamide) (see FIG. 1A) structure was obtained from U.S. Pat. No. 9,884,876 and synthesized de novo by Medchem Source, Inc. for use in the present studies. Working stocks of Sendai virus (SerV) strain Cantell were generated as previously described (Loo, Y .M., et al. (2008). Distinct RIG-I and MDA5 signaling by RNA viruses in innate immunity. Journal of virology 82, 335-345). Mirus Trans-IT mRNA transfection reagent was used treatment of cells with X-RNA and PAMP-RNA. Cyclosporin A (C1832) and Entecavir (SML1103) were obtained from Sigma Aldrich.

In vitro transcription: The poly-U/UC PAMP-RNA and X-RNA were each synthesized from T7 promoter-linked complementary oligonucleotides for the poly-U/UC PAMP RNA (Forward: 5′-TAATACGACTCACTATAGGCCATCCTGTTTTTTTCCCTTTTTTTTTTTCTTTTTTT TTTTTTTTTTTTTTTTTTTTTTTTTTTCTCCTTTTTTTTTCCTCTTTTTTTCCTTTTC TTTCCTTT-3′ (SEQ ID NO:30), Reverse: 5′-AAAGGAAAGAAAAGGAAAAAAAGAGGAAAAAAAAAGGAGAAAAAAAAAAA AAAAAAAAAAAAAAAAAAAAAAAGAAAAAAAAAAAGGGAAAAAAAcAGGA TGGCCTATAGTGAGTCGTATTA-3′ (SEQ ID NO:31)) and X-RNA (Forward: 5′-TAATACGACTCACTATAGGRGGCTCCATCTTAGCCCTAGTCACGGCTAGCTGT GAAAGGTCCGTGAGCCGCTTGACTGCAGAGAGTGCTGATACTGGCCTCTCTGC AGATCAAGT-3′ (SEQ ID NO 32), Reverse: 5′-ACTTGATCTGCAGAGGCCAGTATCAGCACTCTCTGCAGTCAAGCGGCTCAC GGACCTTTCACAGCTAGCCGTGACTAGGGCTAAGATGGAGCCACCTATAGTG AGTCGTATTA-3′ (SEQ D NO:33)) as previously described (Kell, A., et al. (2015, Pathogen-Associated Molecular Pattern Recognition of Hepatitis C Virus Transmitted/Founder Variants by RIG-I Is Dependent on U-Core Length, Journal of Virology 89, 11056-11068; Saito, T., et al. (2008), supra). RNA products were generated by using T7 RNA polymerase and T7 MEGAshortscript kit (Ambion) according to the manufacturers instructions 10 μg of oligonucleotide mixture were annealed using gradient PCR program (95° C. 2 min, with gradual temperature decrease by 1° C./30 sec to 50° C.) After annealing, the reaction mixture was assembled in an RNase-Free micro-centrifuge tube with 7.5 mM of each nucleotide, 10× Reaction buffer, 2 μg, of template DNA and T7 enzyme as described by manufacturer, and the reaction was incubated at 37° C. for 4 hours to allow in vitro transcription. DNA templates were then removed with Turbo DNase treatment and unincorporated nucleotides and protein were removed by phenol-chloroform extraction. RNAs were precipitated by using ethanol and ammonium acetate as described by the manufacturer and resuspended in nuclease-free water. RNA concentrations were determined by absorbance using a Nanodrop spectrophotometer. RNA quality and purity were assessed on denaturing 2% formaldehyde agarose gels.

Reverse transcription quantitative real time qPCR (RT-qPCR) analysis: Total cellular RNAs were extracted from cells using TRIZOL reagent and the manufacturers protocol (Invitrogen). cDNA was synthesized from the purified RNA by both random and oligo (dT) priming using iScript select cDNA synthesis kit (Biorad, Inc.). For HBV cccDNA expression analysis, total DNA was extracted using the DNeasy kit (QIAGEN). For selective cccDNA PCR analysis, isolated DNAs were treated with 10 Units of T5 exonuclease (NEB) for 30 min in 10 μl of reaction volume in 37° C., followed by heat-inactivation at 95° C. for 5 min and 4-fold dilution with Nuclease-free water. For extra-cellular HBV DNA quantification, an external HBV plasmid standard was used to program the PCR (Ko, C., et al. (2018), supra). Relative mRNA levels of all target genes were quantified by RT-qPCR performed using the ΔΔCT method, and expression levels were normalized to house-keeping genes. Real-time PCR assays were carried out using the SYBR green method (Applied Biosystems) performed using an Applied Biosystems 7300 thermocycler. Primer sequence information for RT-qPCR analysis of human and HBV genes is provided in TABLE 3.

Southern Blot analysis of HBV DNA: Southern blot analysis as performed on DNA isolated from cytoplasmic viral capsids exactly as previously described (Ko, C., et al. (2014). DDX3 DEAD-Box RNA Helicase Is a Host Factor That Restricts Hepatitis B Virus Replication at the Transcriptional Level. Journal of Virology 88, 13689-13698; Ko, C., et al. (2014b), Residues Arg703, Asp777, and Arg781 of the RNase H Domain of Hepatitis B Virus Polymerase Are Critical for Viral DNA Synthesis. Journal of Virology 88, 154-163). To detect protein-free forms of HBV-DNA including cccDNA, a modified Hirt extraction method was used, as previously described (Cai, D., et al. (2013). A southern blot assay for detection of hepatitis B virus covalendy closed circular DNA from cell cultures. Methods in Molecular Biology (Clifton, N.J.) 1030, 151-161; Guo, H., et al. (2007). Characterization of the Intracellular Deproteinized Relaxed Circular DNA of Hepatitis B Virus: an Intermediate of Covalently Closed Circular DNA Formation. Journal of Virology 81, 12472-12484). The Hirt extracted protein-free DNAs preparation was digested with plasmid-safe ATP-dependent DNase (Epicentre). The extracted Viral DNA forms were separated on 1.2% agarose gel, transferred to positive charged nylon membrane (GE healthcare, Amersham) via upward capillary transfer, then hybridized with digoxigenin-labeled HBV-specific DNA probe. DNA signal was detected by DIG luminescent detection kit (Roche).

Immunoblot analysis: Immunoblot analysis was performed essentially as described (Lee, S., et al. (2016), Hepatitis B virus X protein enhances Myc stability by inhibiting SCF(Skp2) ubiquitin E3 ligase-mediated Myc ubiquitination and contributes to oncogenesis. Oncogene 35, 1857-1867). Cells were lysed with RIPA buffer containing 0.1% sodium dodecyl sulfate in the presence of protease and phosphatase inhibitor cocktail (Sigma Aldrich). Lysates were separated by SDS-PAGE followed by electrical transfer onto nitrocellulose membranes. The membranes were probed overnight at 4° C. using the appropriate primary antibodies and followed by the corresponding HRP-conjugated secondary antibodies. The following primary antibodies were used for this study: Rabbit anti-IRF3 phosphoserine 386 (Cell Signaling), Rabbit anti-IRF3 (Cell Signaling), Rabbit anti-IFIT1 (antibody 972; raised in rabbit against as IFIT1 437-490 aa peptide sequence), Rabbit anti-RIG-I (antibody 969; raised in rabbit against RIG-I an 1-227 peptide sequence), Rabbit anti-MDA5 (Enzo Life Sciences), Rabbit anti-Lamin B1 (Abeam), Mouse anti-Calnexin (Abeam) and Mouse anti-a-Tubulin (Cell signaling).

Immunofluorescence analysis: Immunofluorescence analysis was performed essentially as described (Lee, S., et al. (2016), supra). Briefly, cells seeded on collagen coated 24-mm coverslips were fixed with 3% paraformaldehyde and permeabilized with 0.2% Triton-X 100 in PBS. Cells were then incubated with mouse monoclonal antibody ARI specific to IRF3 (Rustagi, A., et al. (2013). Two new monoclonal antibodies for biochemical and flow cytometric analyses of human interferon regulatory factor-3 activation, turnover, and depletion. Methods (San Diego, Calif.) 59, 225-232)1 and Rabbit anti-human NTCP (Invitrogen), followed by Alexa Flour 594-, or 488-conjugated specific secondary antibody, respectively (Invitrogen) and 4′,6-diamidino-2-phenylindole (DAPI) incubation. After immunostaining, coverslips were mounted with prolong Gold anti-fade reagent (Life Technologies) and images collected by Nikon Elipse-Ti confocal microscopy.

Cytotoxicity assays: Cytotoxicity was evaluated from cultures of HepG2-C3A -hNTCP and dHepaRG cells using CellTiter-Glo as described (Edwards, T. C., et al. (2019). Inhibition of HBV replication by N-hydroxvisoquinolinedione and N-hydroxypyridinedinone ribonuclease H inhibitors. Antiviral research 164, 70-80). Cells were seeded in 96-well culture plates in DMEM medium and incubated in the presence or absence of serially diluted compound or poly-U/UC PAMP. Cytotoxicity of each was measured with the ATP content as a measure of cell viability using the CellTiter-Glo™ reagent (Promega) per manufacturer's instructions. The plates were read using a luminescence plate reader (Berthold) and the relative luminescence unit (RLU) data generated from each well was calculated as percent signal compared to the untreated control. And values were expressed as CC₅₀ values (50% cytotoxic concentration; the concentration of compound or poly-U/UC PAMP resulting in 50% reduction of absorbance compared to untreated cells, respectively). Tests were carried out in triplicate and each experiment was repeated three tunes. For the purpose of calculating selectivity index (SI), CC₅₀ values greater than 40 were assigned the maximum value of 40. The selectivity index (SI) of compound was calculated as followed: SI=CC₅₀/IC₅₀.

HBV entry assay and cell fractionation: Cells were inoculated with HBV in presence of 4% PEG for 6 hours at 4°C. To assess HBV entry, inoculum was removed by washing with PBS that included proteinase K and cells were shifted to 37° C. post-attachment. After incubation and treatment, the cells were lysed in a hypotonic buffer (100 MM HEPES, 15 mM MgCl₂, 100 mM KCL and Nonidet P-40), and homogenized with a dounce homogenizer. The cytoplasmic fraction was separated from nuclei pellet by centrifugation (6000×g for 5 min at 4° C.). Nuclei pellet was resuspended in extraction buffer (20 mM HEPES, 15 mM MgCl₂, 420 mM NaCl, 0.2 mM EDTA and 25% (v/v) Glycerol including DTI and protease inhibitor cocktail). Each cellular fraction was mixed with 1% SDS (v/v) and protein-free DNA was extracted using the Hirt extraction method.

ELISA: For ELISA, supernatant from cells were collected, centrifuged at 10,000×g for 5 minutes, and liquid fraction recovered for analysis. HBsAg HASA were performed on the recovered supernatant using the Hepatitis B virus s Antigen (HBsAg) Detection Kit (AlphaLISA; PerkinElmer) following the manufacturer's instructions.

cccDNA half-life analysis model: To analyze the decay of cccDNA under treatment, the following mathematical model was employed: C(t)=C(0) if t≤τ otherwise C(t)=C(0)e^{-λ(t-τ)}, where C(t) is the amount of cccDNA at time t post-treatment, C(0) is the cccDNA at the start of treatment, λ is the rate of decay of cccDNA and τ is the delay before therapy causes a decay in cccDNA. By simultaneously fitting the data from the three replicates under each of the four treatments, λ and τ were estimated using MATLAB R2017b. The half-life of cccDNA is calculated as ln (2)/λ and reported in TABLE 2. Using the function nlparci in MATLAB, which is based on the method of asymptotic normal approximation of the least squares estimator (Vandeginste, B. (1989). Nonlinear regression analysis: Its applications, D. M. Bates and D. G. Watts, Wiley, New York, 1988, ISBN 0471-816434. Price: £34.50. Journal of Chemometrics 3 ,544-545), the 95% confidence interval of the parameters λ and τ were estimated and reported TABLE 2.

Statistical analysis: Statistical analyses were performed using Graphpad software with multiple comparison. Continuous variable was reported as mean±standard deviation (SD). For all tests, p values≤0.05 were considered as statistically significant.

While illustrative embodiments have been illustrated and described, it will be appreciated that various changes can be made therein without departing from the spirit and scope of the invention.

1. A method for suppressing hepatitis B virus (HBV) covalently-closed -circular DNA (cccDNA) levels in an infected cell, comprising contacting the infected cell with an agent that induces interferon regulatory factor 3 (IRF3) activation in the infected cell.

2. The method of claim 1, wherein suppressing cccDNA comprises inhibiting cccDNA formation in the infected cell,

3. The method of claim 1, wherein suppressing cccDNA comprises reducing the stability of existing cccDNA in the infected cell.

4. The method of one of claims 1-3, wherein the agent induces IRF3 activation by inducing a retinoic acid-inducible gene I (RIG-I)-like receptor (RLR) signaling pathway.

5. The method of claim 4, wherein the RLR signaling pathway comprises RIG-I, melanoma differentiation-associated gene 5 (MDA5), laboratory of genetics and physiology 2 (LGP2) and/or mitochondrial antiviral signaling (MAVS) protein.

6. The method of one of claims 1-5, wherein the agent is or comprises a nucleic acid molecule comprising a pathogen-associated molecular pattern (PAMP), wherein the PAW comprises:
a 5′-arm region comprising a terminal triphosphate;
a poly-uracil core comprising at least 8 contiguous uracil residues; and
a 3′-arm region comprising at least 8 nucleic acid residues, wherein the 5′-most nucleic acid residue of the 3′-arm region is not a uracil and wherein the 3′-arm region is at least 30% uracil residues.

7. The method of claim 6, wherein the pule uracil core consists of between 8 and 30 uracil residues.

8. The method of claim 6, wherein the 5′-most nucleic acid residue of the 3′-arm region is a cytosine residue or a guanine residue.

9. The method of claim 6, wherein the 3′-arm region is at least 90% uracil residues.

10. The method of claim 6, wherein the 3′-arm region comprises at least 7 contiguous uracil residues.

11. The method of claim 6, wherein the 5′-arm region further comprises one or more nucleic acid residues disposed between the terminal triphosphate and the poly-uracil core.

12. The method of claim 6, wherein the 5′-arm region consists of the terminal triphosphate, and wherein the terminal triphosphate is linked directly to the 5′-end of the poly-uracil core.

13. The method of claim 6, wherein the nucleic acid molecule comprises a sequence of at least 16 nucleotides.

14. The method of one of claims 1-5, wherein the agent is a small molecule agent.

15. The method of claim 14, wherein the small molecule agent is or comprises a benzothiazol-derivative molecule.

16. The method of claim 15, wherein the small molecule agent comprises the chemical formula N-(6-benzamido-1,3-benzothiazol-2-yl)naphthalene-2-carboxamide.

17. The method of claim 1, comprising contacting the infected cell with two or more agents that induce IRF3 activation in the infected cell.

18. The method of claim 17, wherein the two or more agents comprise:
a nucleic acid molecule comprising:
a 5′-arm region comprising a terminal triphosphate;
a poly-uracil core comprising at least 8 contiguous uracil residues; and
a 3′-arm region comprising at least 8 nucleic acid residues, wherein the 5′-most nucleic acid residue of the 3′-arm region is not a uracil and wherein the 3′-arm region is at least 30% uracil residues; and
a small molecule agent is or comprises a benzothiazol-derivative molecule, such as comprising the chemical formula N-(6-benzamido-1.3-benzothiazol-2-yl)naphthalene-2 -carboxamide.

19. The method of claim 1, further comprising contacting the cell with a nucleoside reverse transcriptase inhibitor (NRTI).

20. The method of claim 19, wherein the NRTI is selected from Lamivudine, Adefovir, dipivoxil, Entecavir, Telbivudine, Tenofovir, Tenofovir alafenamide (TAF), Clevudine, Besivo, Zadaxin, Remdesivir, and the like.

21. The method of claim 1, wherein the agent is or comprises a nucleic acid molecule comprising a pathogen-associated molecular pattern (PAMP), wherein the PAMP comprises:
a 5′-arm region comprising a terminal triphosphate;
a poly-uracil core comprising at least 8 contiguous uracil residues; and
a 3′-arm region comprising at least 8 nucleic acid residues, wherein the 5′-most nucleic acid residue of the 3′-arm region is not a uracil and wherein the 3′-arm region is at least 30% uracil residues;
wherein the method further comprises contacting the cell with an NRTI selected from Lamivudine, Adefovir dipivoxil, Entecavir, Telbivudine, Tenofovir, Tenofovir alafenamide (TAF), Clevudine, Besivo, Zadaxin, Remdesivir, and the like.

22. The method of any preceding claim, wherein the infected cell is a hepatocyte.

23. A method of treating or preventing a hepatitis B virus (HBV) infection in a subject in need thereof, comprising administering to the subject a therapeutically effective amount of composition that induces interferon regulatory factor 3 (IRF3) activation in infected cells of the subject.

24. The method of claim 23, wherein the composition is or comprises a nucleic acid molecule comprising a pathogen-associated molecular pattern (PAMP), wherein the PAMP comprises:
a 5′-arm region comprising a terminal triphosphate;
a poly-uracil core comprising at least 8 contiguous uracil residues: and
a 3′-arm region comprising at least 8 nucleic acid residues, wherein the 5′-most nucleic acid residue of the 3′-arm region is not a uracil and wherein the 3′-arm region is at least 30% uracil residues.

25. The method of claim 24, wherein the poly-uracil core consists of between 8 and 30 uracil residues.

26. The method of claim 24, wherein the 5′-most nucleic acid residue of the 3′-arm region is a cytosine residue or a guanine residue.

27. The method of claim 24, wherein the 3′-arm region is at least 90% uracil residues.

28. The method of claim 24, wherein the 3′-arm region comprises at least 7 contiguous uracil residues.

29. The method of claim 24, wherein the 5′-arm region further comprises one or more nucleic acid residues disposed between the terminal triphosphate and the poly-uracil core.

30. The method of claim 24, wherein the 5′-arm region consists of the terminal triphosphate, and wherein the terminal triphosphate is linked directly to the 5′-end of the poly-uracil core.

31. The method of claim 24, wherein the nucleic acid molecule comprises a sequence of at least 16 nucleotides.

32. The method of claim 23, wherein the composition is or comprises a small molecule agent that induces RIG-I signaling.

33. The method of claim 32, wherein the agent is or comprises a benzothiazol-derivative molecule.

34. The method of claim 33, wherein the small molecule agent comprises the chemical formula (N-(6-benzamido-1,3-benzothiazol-2-yl)naphthalene-2-carboxamide).

35. The method of claim 23, comprising administering to the subject therapeutically effective amounts of a first agent and a second agent,
wherein the first agent is or comprises a nucleic acid molecule comprising:
a ′-arm region comprising a terminal triphosphate;
a poly-uracil core comprising at least 8 contiguous uracil residues; and
3′-arm region comprising at least 8 nucleic acid residues, wherein the 5′-most nucleic acid residue of the 3′-arm region is not a uracil and wherein the 3′-arm region is at least 30% uracil residues;
wherein the second agent is or comprises a small molecule agent comprising the chemical formula N-(6-benzamido-1,3-benzothiazol-2-yl)naphthene-2-carboxamide.

36. The method of one of claims 23-35, further comprising administering to the subject a therapeutically effective amount of a nucleoside reverse transcriptase inhibitor (NRTI).

37. The method of claim 36, wherein the NRTI is selected from Lamivudine, Adefovir dipivoxil, Entecavir, Telbivudine, Tenofovir, Tenofovir alafenamide (TAF), Clevudine, Besivo, Zadaxin, Remdesivir, and the like.

38. The method of claim 23, comprising administering to the subject therapeutically effective amounts of a first agent and a second agent,
wherein the first agent is or comprises a nucleic acid molecule comprising:
a 5′-arm region comprising a terminal triphosphate;
a poly-uracil core comprising at least 8 contiguous uracil residues; and
a 3′-arm region comprising at least 8 nucleic acid residues, wherein the 5′-most nucleic acid residue of the 3′-arm region is not a uracil and wherein the 3′-arm region is at least 30% uracil residues; and
wherein the second agent is or comprises an-NRTI.

39. The method of claim 38, wherein the NRTI is selected from Lamivudine, Adefovir dipivoxil, Entecavir, Telbivudine, Tenofovir, Tenofovir alafenamide (TAF), Clevudine, Besivo, Zadaxin, Remdesivir, and the like.

40. A composition for treating a hepatitis B virus (HBV) infection in a subject comprising:
a RIG-I agonist,
a vehicle for intracellular delivery, and
a pharmaceutically acceptable carrier.

41. The composition of claim 40, wherein the RIG-I agonist is or comprises a nucleic acid molecule comprising a pathogen-associated molecular pattern (PAMP), wherein the PAMP comprises:
a 5′-arm region comprising a terminal triphosphate;
a poly-uracil core comprising at least 8 contiguous uracil residues; and
a 3′-arm region comprising at least 8 nucleic acid residues, wherein the 5′-most nucleic acid residue of the 3′-arm region is not a uracil and wherein the 3′-arm region is at least 30% uracil residues.

42. The composition of claim 41, wherein the poly-uracil core consists of between 8 and 30 uracil residues.

43. The composition of claim 41, wherein the 5′-most nucleic acid residue of the 3′-arm region is a cytosine residue or a guanine residue.

44. The composition of claim 41, wherein the 3′-arm region is at least 90% uracil residues.

45. The composition of claim 41, wherein the 3′-arm region comprises at least 7 contiguous uracil residues.

46. The composition of claim 41, wherein the 5′-arm region further comprises one or more nucleic acid residues disposed between the terminal triphosphate and the poly-uracil core.

47. The composition of claim 41, wherein the 5′-arm region consists of the terminal triphosphate, and wherein the terminal triphosphate is linked directly to the 5′-end of the poly-uracil core.

48. The composition of claim 41, wherein the nucleic acid molecule comprises a sequence of at least 16 nucleotides.

49. The composition of claim 40, the RIG-I agonist is or comprises a henzothiazol-derivative molecule, such as comprising the chemical formula N-(6-benzamido-1,3-benzothiazol-2-yl)naphthalene-2-carboxamide.

50. The composition of one of claims 40-49, further comprising a nucleoside reverse transcriptase inhibitor (NRTI).

51. The composition of claim 50, wherein the NRTI is selected from Lamivudine, Adefovir dipivoxil, Entecavir, Telbivudine, Tenofovir, Tenofovir alafenamide (TAF), Clevudine, Zadaxin, Remdesivir, and the like

52. The composition of one of claims 40-51, wherein the vehicle wherein the RIG-I agonist is incorporated into the vehicle.

53. The composition of one of claims 40-51, wherein the vehicle is a liposome, nanocapsule, nanoparticle, exosome, microparticle, microsphere, lipid particle, vesicle, and the like, configured for the introduction of the RIG-I agonist into target host cells infected with HBV.

54. A method of treating a subject with a hepatitis B virus (HBV) infection, comprising administering to the subject a therapeutically effective amount of the composition of one of claims 40-53.


SEQUENCE LISTING (125 sequences)

SEQ ID NO: 1 - RNA, 105 nt, Artificial sequence
  Synthetic
ggccauccuguuuuuuucccuuuuuuuuuuucuuuuuuuuuuuuuuuuuuuuuuuuuuuuuuuuuucuccuuuuuuuuuccucuuuuuuuccuuuucuuuccuuu

SEQ ID NO: 2 - DNA, 22 bp, Artificial sequence
  Synthetic
gcctattgattggaaagtatgt

SEQ ID NO: 3 - DNA, 16 bp, Artificial sequence
  Synthetic
gctgaggcggtatcta

SEQ ID NO: 4 - DNA, 19 bp, Artificial sequence
  Synthetic
ctcctccagcttatagacc

SEQ ID NO: 5 - DNA, 17 bp, Artificial sequence
  Synthetic
gtgagtgggcctacaaa

SEQ ID NO: 6 - DNA, 22 bp, Artificial sequence
  Synthetic
agaagcaggcaatcacagaaaa

SEQ ID NO: 7 - DNA, 23 bp, Artificial sequence
  Synthetic
ctgaaaccgaccatagtggaaat

SEQ ID NO: 8 - DNA, 23 bp, Artificial sequence
  Synthetic
tactccgtgaagtctagggacag

SEQ ID NO: 9 - DNA, 22 bp, Artificial sequence
  Synthetic
aacaggatgaatccaatggtca

SEQ ID NO: 10 - DNA, 21 bp, Artificial sequence
  Synthetic
gtggcattcaaggagtacctc

SEQ ID NO: 11 - DNA, 22 bp, Artificial sequence
  Synthetic
tgatggccttcgattctggatt

SEQ ID NO: 12 - DNA, 20 bp, Artificial sequence
  Synthetic
cgtgagcatcgtgagcaatg

SEQ ID NO: 13 - DNA, 20 bp, Artificial sequence
  Synthetic
tcttctttccttggccacgg

SEQ ID NO: 14 - DNA, 20 bp, Artificial sequence
  Synthetic
tgcaccgaactctaccagca

SEQ ID NO: 15 - DNA, 21 bp, Artificial sequence
  Synthetic
gtttctcccaccctctcctcc

SEQ ID NO: 16 - DNA, 19 bp, Artificial sequence
  Synthetic
aaggactgcaagtgccgct

SEQ ID NO: 17 - DNA, 18 bp, Artificial sequence
  Synthetic
gctggtccaagacatccc

SEQ ID NO: 18 - DNA, 19 bp, Artificial sequence
  Synthetic
tcacaggcgcattactgcc

SEQ ID NO: 19 - DNA, 21 bp, Artificial sequence
  Synthetic
ggatttgaaccaatcgctgga

SEQ ID NO: 20 - DNA, 20 bp, Artificial sequence
  Synthetic
gagaagggacaagcacatgg

SEQ ID NO: 21 - DNA, 20 bp, Artificial sequence
  Synthetic
tggatccatcaagtgtctgg

SEQ ID NO: 22 - DNA, 19 bp, Artificial sequence
  Synthetic
ccgaggacccgaaggttac

SEQ ID NO: 23 - DNA, 20 bp, Artificial sequence
  Synthetic
tccaacagtgctgaaattcg

SEQ ID NO: 24 - DNA, 22 bp, Artificial sequence
  Synthetic
acaactttggtatcgtggaagg

SEQ ID NO: 25 - DNA, 19 bp, Artificial sequence
  Synthetic
gccatcacgccacagtttc

SEQ ID NO: 26 - DNA, 26 bp, Artificial sequence
  Synthetic
ccccacaaaccccattactaaaccca

SEQ ID NO: 27 - DNA, 26 bp, Artificial sequence
  Synthetic
tttcatcatgcggagatgttggatgg

SEQ ID NO: 28 - DNA, 20 bp, Artificial sequence
  Synthetic
gggtcttccggatataatcc

SEQ ID NO: 29 - DNA, 21 bp, Artificial sequence
  Synthetic
gtggttggactcgggaattcg

SEQ ID NO: 30 - DNA, 122 bp, Artificial sequence
  Synthetic
taatacgactcactataggccatcctgtttttttccctttttttttttcttttttttttttttttttttttttttttttttttctcctttttttttcctctttttttccttttctttccttt

SEQ ID NO: 31 - DNA, 122 bp, Artificial sequence
  Synthetic
aaaggaaagaaaaggaaaaaaagaggaaaaaaaaaggagaaaaaaaaaaaaaaaaaaaaaaaaaaaaaaaaaagaaaaaaaaaaagggaaaaaaacaggatggcctatagtgagtcgtatta

SEQ ID NO: 32 - DNA, 115 bp, Artificial sequence
  Synthetic
taatacgactcactataggtggctccatcttagccctagtcacggctagctgtgaaaggtccgtgagccgcttgactgcagagagtgctgatactggcctctctgcagatcaagt

SEQ ID NO: 33 - DNA, 115 bp, Artificial sequence
  Synthetic
acttgatctgcagagaggccagtatcagcactctctgcagtcaagcggctcacggacctttcacagctagccgtgactagggctaagatggagccacctatagtgagtcgtatta

SEQ ID NO: 34 - RNA, 105 nt, Artificial sequence; identical to SEQ ID NO: 1
  Synthetic

SEQ ID NO: 35 - RNA, 107 nt, Artificial sequence
  Synthetic
acuguuccuuuuuuuuuuuuuuuuuuuuuuuuuuuuuuuuuuuuuuuuuuucuuuuuuuuuuuuuucccucuuucuucccuucucaucuuauucuacuuucuuucuu

SEQ ID NO: 36 - RNA, 131 nt, Artificial sequence
  Synthetic
ggccauccuguuuuuuucccuuuuuuuuuuucuuuuuuuuuuuuuuuuuuuuuuuuuuuuuuuuuucuccuuuuuuuuuccucuuuuuuuccuuuucuuuccuuucccccccccccccccccccccccccc

SEQ ID NO: 37 - RNA, 105 nt, Artificial sequence
  Synthetic
ggccauccuguuuuuuucccuuuuuuuuuuucuuuuuuuuuuuuuuuuuuuuuuuuuuuuuuuuuucuccuuuuuuuuucccccccccccccccccccccccccc

SEQ ID NO: 38 - RNA, 105 nt, Artificial sequence
  Synthetic
ggccauccuguuuuuuucccuuuuuuuuuuucuuuuuuuuuuuuuuuuuuuuuuuuuuuuuuuuuuuuccuuuuuuuuuccucuuuuuuuccuuuucuuuccuuu

SEQ ID NO: 39 - RNA, 105 nt, Artificial sequence
  Synthetic
uuuuuuuuuuuuuuuuuuucuuuuuuuuuuucuuuuuuuuuuuuuuuuuuuuuuuuuuuuuuuuuuvuuuuuuuuuuuuuuuuuuuuuuuuuuuuuuuuuuuuuu

SEQ ID NO: 40 - RNA, 105 nt, Artificial sequence
  Synthetic
ggccauccuguuuuuuucccuuuuuuuuuuucuuuuuuuucuccuuuuuuuuuccucuuuuuuuccuuuucuuuccuuucccccccccccccccccccccccccc

SEQ ID NO: 41 - RNA, 105 nt, Artificial sequence
  Synthetic
ggccauccuguuuuuuucccuuuuuuuuuuucuuuuuuuuuuuuuuuuucuccuuuuuuuuuccucuuuuuuuccuuuucuuuccuuuccccccccccccccccc

SEQ ID NO: 42 - RNA, 62 nt, Artificial sequence
  Synthetic
ggccauccuguuuuuuuuuuuuuuuuuuuuuuuuuuuuuuuuuucuccuuuuuuuuuccucu

SEQ ID NO: 43 - RNA, 62 nt, Artificial sequence
  Synthetic
ccccccccccuuuuuuuuuuuuuuuuuuuuuuuuuuuuuuuuuucuccuuuuuuuuuccucu

SEQ ID NO: 44 - RNA, 62 nt, Artificial sequence
  Synthetic
ggccauccuguuuuuuuuuuuuuuuuuuuuuuuuuuuuuuuuuucccccccccccccccccc

SEQ ID NO: 45 - RNA, 62 nt, Artificial sequence
  Synthetic
uuuuuuuuuuuuuuuuuuuuuuuuuuuuuuuuuuuuuuuuuuuuvuuuuuuuuuuuuuuuuu

SEQ ID NO: 46 - RNA, 62 nt, Artificial sequence
  Synthetic
uuuuuuuuuuuuuuuuuuuuuuuuuuuuuuuuuuuuuuuuuuuucuuuuuuuuuuuuuuuuu

SEQ ID NO: 47 - RNA, 62 nt, Artificial sequence
  Synthetic
uuccuuccuuuuuuuuuuuuuuuuuuuuuuuuuuuuuuuuuuuucuccuuuuuuuuuccucu

SEQ ID NO: 48 - RNA, 62 nt, Artificial sequence
  Synthetic
uuuuuuuuuguuuuuuuuuuuuuuuuuuuuuuuuuuuuuuuuuucuccuuuuuuuuuccucu

SEQ ID NO: 49 - RNA, 62 nt, Artificial sequence; identical to SEQ ID NO: 48
  Synthetic

SEQ ID NO: 50 - RNA, 62 nt, Artificial sequence
  Synthetic
gguuuuccuuuuuuuuuuuuuuuuuuuuuuuuuuuuuuuuuuuucuuuuuuuuuuuuuuuuu

SEQ ID NO: 51 - RNA, 62 nt, Artificial sequence
  Synthetic
ggccauccuguuuuuuuuuucuuuuuuuuuucuuuuuuuuuucucuccuuuuuuuuuccucu

SEQ ID NO: 52 - RNA, 62 nt, Artificial sequence
  Synthetic
ggccauccuguuuuuuuuuuuuuuucuuuuuuuuuuuuuuucuucuccuuuuuuuuuccucu

SEQ ID NO: 53 - RNA, 62 nt, Artificial sequence
  Synthetic
ggccauccuguuuuuuuuuucccuuuuuuuuuucccuuuuuuuucuccuuuuuuuuuccucu

SEQ ID NO: 54 - RNA, 62 nt, Artificial sequence
  Synthetic
ggccauccuguuuuuuuuuuuuuuuuuuccccccuuuuuuuuuucuccuuuuuuuuuccucu

SEQ ID NO: 55 - RNA, 105 nt, Artificial sequence; identical to SEQ ID NO: 1
  Synthetic

SEQ ID NO: 56 - RNA, 35 nt, Artificial sequence
  Synthetic
ggccauucuuuuuuuuuuuuuuuucuuucuucuuu

SEQ ID NO: 57 - RNA, 116 nt, Artificial sequence
  Synthetic
ggccauucccuuuuuuuuuuuuuuuuuuuuuuuuuuuuuuuuuuuuuuuuuuuuuuuuuuuuuuuuuuuuuuuuuuuuuuuuuuuuuuuuucucuucuuuucuuuauuccuucuuu

SEQ ID NO: 58 - RNA, 101 nt, Artificial sequence
  Synthetic
ggccguuccuuuuuuuuuuuuuuuuuuuuuuuuuuuuuuuuuuuuuuuuuuuuuuuuuuuuuuuuuuuuuuuucuuuuccccuuuuuuauuuuucuuucuu

SEQ ID NO: 59 - RNA, 105 nt, Artificial sequence
  Synthetic
ggccauccuguuuuuuuguuuuuucuuuuuuuuuuuuuuuuuuuuuuuuuuuuuuuuuuuuuuuuuuucuuuuuuucccuuuuuuuuauuuuauuuucuuuuggu

SEQ ID NO: 60 - RNA, 131 nt, Artificial sequence
  Synthetic
ggccaucccccuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuccucuuuuuuuccuuuucuucuuu

SEQ ID NO: 61 - RNA, 116 nt, Artificial sequence
  Synthetic
ggccguucuguuuuuuuuuuuuuuuuuuuuuuuuuuuuuuuuuuuuuuuuuuuuuuuuuuuuuuuuuuuuuuuuuuuuuuuuuuuuuuuuuuuuuccuuuuuuuuauuccucuucu

SEQ ID NO: 62 - RNA, 123 nt, Artificial sequence
  Synthetic
ggccauccccuuuguuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuuauuucuccuucuuuu

SEQ ID NO: 63 - RNA, 87 nt, Artificial sequence
  Synthetic
ggccauuccuuuuuuuuuuuuuuuuuuuuuuuuucuuuuuuuuuccuuuuuuuuuuuuuuuuuuuuuuuuccuuuucuuucuucuuu

SEQ ID NO: 64 - RNA, 87 nt, Artificial sequence; identical to SEQ ID NO: 63
  Synthetic

SEQ ID NO: 65 - RNA, 96 nt, Artificial sequence
  Synthetic
agccauuuccuguuuuuuuuuuuuuuuuuuuuuuuuuuuucuuuuuuuuuuucuuuccuuuccuucuuuuuuuccuuucuuuuucccuucuuuaau

SEQ ID NO: 66 - RNA, 101 nt, Artificial sequence
  Synthetic
ggccauuuccuguuuuuuuuuuuuuuugguuuuuuuuuuuuuuuuuuuuuuuuuuuuuuuuuuuuuuuccuuuccuucuuuuuuuuuuuuucccucuuuau

SEQ ID NO: 67 - RNA, 87 nt, Artificial sequence
  Synthetic
ggccauuuccuguuuuuuuuuuuuuuuuuuuuuuuuuuuuuuuuuucuuuuccuucuuuuucccuuuuucuuucuuccuucuuuaau

SEQ ID NO: 68 - RNA, 100 nt, Artificial sequence
  Synthetic
ggccauccuguguuuuuuuuuuuuuuuuuuuuuuuuuuuuuuuuuuuuuuuuuuuuuuuuuuuuuuuuuuuuuuuuuuuuuuuuuuuauuuccuuuucuu

SEQ ID NO: 69 - RNA, 109 nt, Artificial sequence
  Synthetic
ggccaaccuguuuuuuuuuuuuuuuuuuuuuuuuuuccuuuuuuuuuuuuuuuuuuuuuuuuuuuuuuuuuuccuuuuuuucuuuuuuuuuuuuuuuuuccuuccuuuu

SEQ ID NO: 70 - RNA, 34 nt, Artificial sequence
  Synthetic
ggccauccuguuuuuuuuuuuuuuuucuuucuuu

SEQ ID NO: 71 - RNA, 74 nt, Artificial sequence
  Synthetic
ggccauuuuuccuuuuuuuuuuuuuuuuuuuuuuucuuuuuuuuuuccuuuuuuucuuuuuuuuucuuuucuuu

SEQ ID NO: 72 - RNA, 81 nt, Artificial sequence
  Synthetic
ggccauucuuuuuuuuuuuuuuuuuuuuuuuuuuuuuuuuuuucguuucuuuuucuucuuuuuguuuucucuucuccuuuu

SEQ ID NO: 73 - RNA, 107 nt, Artificial sequence
  Synthetic
ggccauuccccuuuuuuuuuuuuuuccgcuuuuuuuuuuuuuuuuuuuuuuuuuuuuuuuuucuuuuuuuuuccuuuuuuuuuuuuuuuuuuuuuuuuuuucuuuuu

SEQ ID NO: 74 - RNA, 109 nt, Artificial sequence
  Synthetic
ggccaucccccuuuuuuuuuuuuuccgcuuuuuuuuuuuuuuuuuuuuucuuuuuuuuuuucuuuuuuuuuuccuuuuuuuuuuuuuuuuuuuuuuuucuuuucuuuuu

SEQ ID NO: 75 - RNA, 35 nt, Artificial sequence; identical to SEQ ID NO: 56
  Synthetic

SEQ ID NO: 76 - RNA, 48 nt, Artificial sequence
  Synthetic
ggccauccccuucuuuuuuuuuuuuuuuuuuuuuuccuuuucuucuuu

SEQ ID NO: 77 - RNA, 100 nt, Artificial sequence
  Synthetic
ggccauccuguuuuuuucccuuuuuuuuuuucuuuuuuuuuuuuuuuuuuuuuuuuuuuuucuccuuuuuuuuuccucuuuuuuuccuuuucuuuccuuu

SEQ ID NO: 78 - RNA, 112 nt, Artificial sequence
  Synthetic
ggccguccuguuuuuuuuuuuuuuuuuuuccuuuuuuuuuuuuuuuuuuuuuuuuuuuuuuuuuuuuuuuuuuuuuuuuuuuuuuuuuuuuuuuuuuucuucuuucuuucuu

SEQ ID NO: 79 - RNA, 145 nt, Artificial sequence
  Synthetic
ggccauuuccuguuuuuuuuuuuuuuuuuuuuuuuuuuuuuuuuuuuuuuuuuuuuuuuuuuuuucuuuuuuuuuuuuuuuuuccuuuuuuuuuuuuuuuuuuuucuuuccuucuuuuuuccuuucuuuuccuuccuucuuuaau

SEQ ID NO: 80 - RNA, 95 nt, Artificial sequence
  Synthetic
ggccauuccuguuuuuuuuuuuuuuuucuuuuguuuuuuuuguuuuuuuuuuuuuuuuuccuuucuuuuuuuuuuuuuuuccuuucuucuuuaau

SEQ ID NO: 81 - RNA, 72 nt, Artificial sequence
  Synthetic
ggccauuuccuguuuuuuuuuuuuuuuuuuuucuuuccuucuuuuuuccuuucuuuuccuuccuucuuuaau

SEQ ID NO: 82 - RNA, 87 nt, Artificial sequence; identical to SEQ ID NO: 67
  Synthetic

SEQ ID NO: 83 - RNA, 143 nt, Artificial sequence
  Synthetic
ggccauuuccuguuuuuuuuuuuuuuuuuuuuuuuuuuuuuuuuuuuuuuuuuuuuuuuuuuucuuuuuuuuuuuuuuuuuccuuuuuuuuuuuuuuuuuuuucuuuccuucuuuuuuccuuucuuuuccuuccuucuuuaau

SEQ ID NO: 84 - RNA, 108 nt, Artificial sequence
  Synthetic
ggccauuuccuguuuuuuuuuuuuuucccuuuuuuuuuuuuuuuuuuuuuuuuuuuuuuuuuuuuucuuuccuucuuuuuuuuccuuucuuuuccuuccuucuuuaau

SEQ ID NO: 85 - RNA, 82 nt, Artificial sequence
  Synthetic
ggcauccuguuuuuuuuuuuuuuuuuuuuuuuuuuuuuuuuuuuuuuuuuuuuuuuuuuuuuuuuuuuuuuuucuuuucuuu

SEQ ID NO: 86 - RNA, 130 nt, Artificial sequence
  Synthetic
acacuccauuucuuuuuuuguuuuuuuuuuuuuuuuuuuuuuuuuuuuuuuuuuuuuuuuuuuuuuuuuuuuuuuuuuuuuuuuuuucuuuuucuuuccuuucuuuucugacuucuaauuuuccuucuua

SEQ ID NO: 87 - RNA, 129 nt, Artificial sequence
  Synthetic
guccuucuguuuuuuuuuuuuuuuuuuuuuuuuuuuuuuuuuuuuuuuuuuuuuuuuuuuuuuuuuuuuuuuuuuuuuuuuuuuuuuccuuacccuuuccuucuuuucuuccuuuuuuuuccuuacuuu

SEQ ID NO: 88 - RNA, 57 nt, Artificial sequence
  Synthetic
ggguccccuuguuuuuuuuuuuucuuuccuucuuuccuuuccuaaucuuucuuucuu

SEQ ID NO: 89 - RNA, 96 nt, Artificial sequence; identical to SEQ ID NO: 65
  Synthetic

SEQ ID NO: 90 - RNA, 115 nt, Artificial sequence
  Synthetic
ggccauuuccuguuuuuuuuuuuuuuuguuuuuuuuuuuuuuuuuuuuuuuuuuuuuuuuuuuuuuuuuuuuuuuuuuuuuuuccuuuccuuuuuuuuuuuuuuucccuuuuuau

SEQ ID NO: 91 - RNA, 107 nt, Artificial sequence
  Synthetic
ggccauccuguuuuuuuuuuuuuuuuuuuuuucuuuuuuuuuuuuuuuuucuuuuuuuuucuucuuuuucuuuccuuuuuuuuuuuuuuuuuuuuuuuucuucuuuc

SEQ ID NO: 92 - RNA, 96 nt, Artificial sequence
  Synthetic
ggccauuuccuguuuuuuuuuuuuuuuuuuuuuuuuuuuuuuuuuuuuuuuuuuuuuucuuuuucccucuuuuucuucucuuuuuccuucuuuaau

SEQ ID NO: 93 - RNA, 111 nt, Artificial sequence
  Synthetic
gcuaacuguuccuuuuuuuuuuuuuuuuuuuuuuuuuuuuuuuuuuuuuuuuuuucuuuuuuuuuuuuuucccucuuucuucccuucucaucuuauucuacuuucuuucuu

SEQ ID NO: 94 - RNA, 107 nt, Artificial sequence
  Synthetic
gcuaacuguuccuuuuuuuuuuuuuuuuuuuuuuuuuuuuuuuuuuuuuucuuuuuuuuuuuuuuucccucuuucuucccuucucaucuuauucuacuuucuuucuu

SEQ ID NO: 95 - RNA, 92 nt, Artificial sequence
  Synthetic
gcuaacuguuccuuuuuuuuuuucuuuuuuuuuuuuuuuuuuuuuuuuuuuccuucuuucuuucuuucuuaccuuacuuuacuuucuuuucu

SEQ ID NO: 96 - RNA, 111 nt, Artificial sequence
  Synthetic
gcuaacaguuucucuuuuuuuuuuuuuccuuuuuuauuuuuauuuuuuuuuuuuuuuuuuuuuuuuuauuuucuuuuccuuucuuucucaccuuacauuacuuucuuucuu

SEQ ID NO: 97 - RNA, 77 nt, Artificial sequence
  Synthetic
gcuaauuuccuuauuguuuuuuuuuuuuuuuuuuucuuuccauuuccuuccuucuuacuucacuuuaccuucuuucu

SEQ ID NO: 98 - RNA, 124 nt, Artificial sequence
  Synthetic
gcuaacuguuuuuuuuuuuuuuuuuuuuuuuuuuuuuuuuuuuuuuuuuuuuuuuuuuuuuuuuuuuuuuuuuuuuuuuuuuuuuccuuuccuuucuuucuuaccuuacuuuacauucuuuucu

SEQ ID NO: 99 - RNA, 119 nt, Artificial sequence
  Synthetic
gcuaacuguuccuuuuuuuuuuuuuuuuuuuuuuuuuuuuuuuuuuuuuuuuuuuuuuuuuuuuuuuuuuuuuuuuuuuuuucuuuccuuccuuucucaccuucuuuuacuucuuuccu

SEQ ID NO: 100 - RNA, 93 nt, Artificial sequence
  Synthetic
gcuaacuguuuuuuuuuuuuuuuuuuuuuuuuuuuuuuuuuuuuuuuuuuuuucuuuucuuuccuuuccuucuuacucuacuuuacuuuuucu

SEQ ID NO: 101 - RNA, 129 nt, Artificial sequence
  Synthetic
gcuaacuguucuuuuuuuuuuuuuuuuuuuuuuuuuuuuuuuuuuuuuuuuuuuuuuuuuuuuuuuuuuuuuuuuuuuuuuuuuuuuuucuuuuccuucuucuuucuuaccuuauuuuccuucuuucuu

SEQ ID NO: 102 - RNA, 86 nt, Artificial sequence
  Synthetic
gcuaacuguuuuuuuuuuuuuuuuuuuuuuuuuuuuuucuuuuuuuuucuuuucuuuccuucuuaccuuacuuuacuuucuuuucu

SEQ ID NO: 103 - RNA, 133 nt, Artificial sequence
  Synthetic
gcuaacuguuuuuuuuuuuuuuuuuuuuuuuuuuuuuuuuuuuuuuuuuuuuuuuuuuuuuuuuuuuuuuuuuuuuuuuuuuuuuuuuuccuuuuuccuuuuccuucucuuuuuaccuuacuuuacuuuucuu

SEQ ID NO: 104 - RNA, 144 nt, Artificial sequence
  Synthetic
gcuaacugucccuuuuuuuuuuuuuuuuuuuuuuuuuuuuuuuuuuuuuuuuuuuuuuuuuuuuuuuuuuuuuuuuuuuuuuuuuuuuuuuuuuuucuuuuuuucucuuuuccuucuuucuuaccuuauuuuacuuucuuuccu

SEQ ID NO: 105 - RNA, 119 nt, Artificial sequence
  Synthetic
gcuaacugucccuuuuuuuuuguuuuuuuuuuuuuuuuuuuuuuuuuuuuuucuuuuuuuuuuuuuuuuuuguuucuuuuccuucucauuuccuucuuaucuuaauuacuuccuuuccu

SEQ ID NO: 106 - RNA, 88 nt, Artificial sequence
  Synthetic
gcuaacuguuuuuuuuuuuuuuuuuuuuuuuuuuuuuuuuuuuuuuuccuucuuccuuuccuucuuaccuuacuuuauuuucuuuccu

SEQ ID NO: 107 - RNA, 101 nt, Artificial sequence
  Synthetic
gcuaacuguuuuuuuuuuuuuuuuuuuuuuuuuuuuuuuuuuuuuuuuuuuuuuuuuuuuuucuuucuuuucuuuucucaccuuacuuuacuuccuuucuu

SEQ ID NO: 108 - RNA, 86 nt, Artificial sequence
  Synthetic
gcuaguuuucuuuuuuuuuuuuuuuuuuuuuuuuguuuuuuuuuuuuuuccucuuuuuccguauuuuuuuuuuuuccucuuuucuu

SEQ ID NO: 109 - RNA, 105 nt, Artificial sequence; identical to SEQ ID NO: 1
  Synthetic

SEQ ID NO: 110 - RNA, 79 nt, Artificial sequence
  Synthetic
ccauuuuucuuuuuuuuuuuuuguuuguuuuuuuuuuuuuuuucuuuccuucuuuccugacuuuuaauuuuccuucuua

SEQ ID NO: 111 - RNA, 116 nt, Artificial sequence
  Synthetic
ccauuuuucuuuuuuuuuuuuuuuuuuuuuuuuuuuuuuuuuuuuuuuuuuuuuuuuuguuuguuuuuuuuuuuuuuuuucuuuccuucuuuccugacuuuuaauuuuccuucuua

SEQ ID NO: 112 - RNA, 84 nt, Artificial sequence
  Synthetic
ggccauuuccuguuuuuuuuuuuuuuuuuuuuuuuuuuuuuuuuuacccuuuuuucucuuuuuuuuuuuuccuucuucuuuaau

SEQ ID NO: 113 - RNA, 74 nt, Artificial sequence
  Synthetic
ggccauuuccuguuuuuuuuuuuuuuuuuuacccuuuuuucucuuuuuuuuuuuuuuuuuccuucuucuuuaau

SEQ ID NO: 114 - RNA, 38 nt, Artificial sequence
  Synthetic
ggccauuuucuguuuuuuuuuuuuuuauuuucuuuaau

SEQ ID NO: 115 - RNA, 53 nt, Artificial sequence
  Synthetic
ggccauuuucuuuuuuuuuucucuuuuuuuuuuuuuuuuuuauuuucuuuaau

SEQ ID NO: 116 - RNA, 82 nt, Artificial sequence
  Synthetic
ggccauuuucuguuuuuuuuuuuuuuuuuuuuccuuuuuuuuuuuuccucuuuuuuuuuuuuuuuuuuuuauuuucuuuaau

SEQ ID NO: 117 - RNA, 82 nt, Artificial sequence
  Synthetic
ggccauuuucuguuuuuuuuuuuucuuuuuuuuuuuuuuuuuccuuuuuuuuucucuuuuuuuuuuuuuuauuuucuuuaau

SEQ ID NO: 118 - RNA, 85 nt, Artificial sequence
  Synthetic
ggccauccuguuuuuuuuuuuuuuuuuuuuuuuuguuuuuuuuuuuuuuuuucuuuuuccuuuuuuuuuuuuuauuuucuucuuu

SEQ ID NO: 119 - RNA, 59 nt, Artificial sequence
  Synthetic
gguccuaaguuuuuuuuuuuuucuuccuuccuucuuuccuuuucuaauuuuccuucuuu

SEQ ID NO: 120 - RNA, 62 nt, Artificial sequence
  Synthetic
gguccuaaguuguuuuuuuuuuuuuuuccuuccuucuuucccuuuucuaauuuuccuucuuu

SEQ ID NO: 121 - RNA, 74 nt, Artificial sequence
  Synthetic
gguccuaaguuguuuuuuuuuuuuuuuuuuuuuuuccuuuccuuccuucuuuccuuuucuaauuuuccuucuuu

SEQ ID NO: 122 - RNA, 95 nt, Artificial sequence
  Synthetic
ggccauuucuguuuuuuuuuuuuuuuuuuuuuuuuuuuuuuuuuuuuuuuuuguuuccuucuuuuuccuuuucuuuuuuuuuuucucccuuuaau

SEQ ID NO: 123 - RNA, 70 nt, Artificial sequence
  Synthetic
ggccauuucuguuuuuuuuuuuuuuguuuccuucuuuuuccuuuucuuuuuuuuuuuuucucccuuuaau

SEQ ID NO: 124 - RNA, 70 nt, Artificial sequence
  Synthetic
ggccauccuguuuuuuucccuuuuuuuuuuucuccuuuuuuuuuccucuuuuuuuccuuuucuuuccuuu

SEQ ID NO: 125 - RNA, 53 nt, Artificial sequence; identical to SEQ ID NO: 115
  Synthetic